(19) United States
(12) Patent Application Publication
(10) Pub. No.: US 2026/0243776 A1
(43) Pub. Date: Aug. 20, 2026

(54) A METHOD FOR DETECTING PROTEIN TARGETS OF AN ENZYME USING A BIOTIN LIGASE

(71) Applicant: Leibniz-Institut für Alternsforschung - Fritz-Lipmann-Institut e.V. (FLI), Jena (DE)

(72) Inventors: Alessandro Ori, Jena (DE); Aleksandar Bartolome, Jena (DE); Joanna M. Kirkpatrick, Jena (DE); Therese Dau, Jena (DE); Julia Heiby, Jena (DE)

(21) Appl. No.: 18/993,990

(22) PCT Filed: Jul. 14, 2023
(86) PCT No.: PCT/EP2023/069680

§ 371 Date: Jan. 13, 2025

(30) Foreign Application Priority Data
Jul. 14, 2022 (DE) 10 2022 117 634.7

(51) Int. Cl. G01N 33/68; A01K 67/0278; C12N 9/00; C12N 15/62; C12N 15/85; C12Q 1/25

(52) U.S. Cl. CPC G01N 33/6848; A01K 67/0278; C12N 9/93; C12N 15/62; C12N 15/85; C12Q 1/25; G01N 33/6845; A01K 2217/203; A01K 2227/105; A01K 2267/0393; C07K 2319/40; C12N 2830/003; C12N 2830/006; C12Y 603/04015; G01N 2333/9015

(57) ABSTRACT

A method of detecting an interaction or proximity between a fusion protein and a target protein. The method may include: a. providing a fusion protein and/or a recombinant nucleic acid molecule encoding said fusion protein, said fusion protein including an enzyme or enzyme complex member fused to a promiscuous biotin ligase, b. inhibiting the enzyme function of the fusion protein or the enzyme complex, c. biotinylating a target protein that interacts with and/or is in proximity to the fusion protein, d. enriching the biotinylated target protein, and identifying the isolated target protein using mass spectrometry (MS). The invention further relates to a fusion protein comprising a proteasome complex protein fused to a promiscuous biotin ligase, a cell line and a mouse expressing said fusion protein and a kit for performing the disclosed methods.

The invention is in the field of proteomics and determination of protein-protein interactions, such as determining an interaction or proximity between two proteins.

The invention relates to a method of detecting an interaction or proximity between a fusion protein and a target protein, comprising a. providing a fusion protein and/or a recombinant nucleic acid molecule encoding said fusion protein, said fusion protein comprising an enzyme or enzyme complex member fused to a promiscuous biotin ligase, b. inhibiting the enzyme function of the fusion protein or the enzyme complex, c. biotinylating a target protein that interacts with and/or is in proximity to the fusion protein, d. enriching the biotinylated target protein, and identifying the isolated target protein using (quantitative) mass spectrometry (MS).

The invention further relates to a fusion protein comprising a proteasome complex protein fused to a promiscuous biotin ligase, a cell line and a mouse expressing said fusion protein and a kit for performing the methods according to the invention.

### BACKGROUND OF THE INVENTION

Enzymes and enzyme complexes, such as proteases or the proteasome, play an important role in various cellular processes including processes relevant for certain diseases and their treatment. Proteases regulate cell fate, protein localization and their lifetime and activity, they regulate protein-protein interactions, and they assist in the generation of cellular information by modulating cellular signaling. Proteases also influence transcription, cell division and differentiation. Importantly, they regulate emergency responses to stressors such as heat shock and unfolded or misfolded proteins. Due to their essential role in regulating healthy as well as emergency functions in each cell, proteases also play an important role in pathological conditions such as cancer, cardio-vascular, inflammatory and neurodegenerative diseases.

An example of important proteolytic enzyme complexes is the proteasome, a large protein complex responsible for degradation of cellular proteins. Polymerized ubiquitin serves a key label, marking proteins for degradation by the proteasome. The degradation of proteins is initiated through covalent attachment of ubiquitin molecules. The proteasome consists of two subcomplexes: a catalytic core particle (also termed the 20S proteasome) and one or two terminal regulatory particle(s), termed 19S that function as activators of the proteasome (termed PA700, due to their molecular mass of ~700 kDa). The 20S proteasome is a structurally highly organized protein complex forming a barrel like shape by axial stacking of two outer alpha-rings and two inner beta-rings. In general substrates are able to access the active sites of the 20S proteasome by passing through the narrow opening at the center of the alpha-rings. The 20S proteasome processivity degrades protein substrates thereby generating oligopeptides ranging between 3 to 15 amino-acids in length. The enzymatically active proteasome is generally closed by regulatory proteins at one or both ends of the central 20S proteasome core, which recognize poly-ubiquitinated substrates, removes the ubiquitin chain and traps the protein part of the substrate to unfolds it. It subsequently opens the alpha-ring, and transfers the unfolded substrates to the 20S core particle for degradation.

The cellular process of degradation of misfolded, damaged or un-needed proteins by the proteasome can also be harnessed for therapeutic purposes. In such proteasome-dependent therapies important mediators of diseases can be tagged for proteasomal degradation and thus be eliminated. This approach is especially interesting for so called un-druggable drivers of disease, such as protein products of oncogenes that could not yet be successfully targeted pharmacologically.

Targeted protein degradation (TPD) as a therapeutic approach has enormous potential as it may be feasible for numerous therapeutic indications. The greatest focus so far has been the development of new drugs for cancer therapy. One approach is to employ a cell's own protein disposal (degradation) system by administering the small compound “proteolysis targeting chimera” (PROTAC) to induce degradation of proteins of choice. Rather than acting as a conventional enzyme inhibitor, a PROTAC works by inducing selective intracellular proteolysis. PROTACs commonly consist of two covalently linked protein-binding molecules: one capable of engaging an E3 ubiquitin ligase, and another that binds to a target protein meant for degradation. This approach enables elimination of otherwise untreatable disease-causing proteins and constitutes a promising method to overcome resistance to traditional drugs, especially small molecule agents. However, slowly the limits and risks of this approach are becoming apparent. Targeted protein degraders (TPDs) like PROTACs are more difficult to develop than traditional small molecules, many types of proteins are not easily degraded, their toxicology is uncertain and side effects are largely unknown. Hence, no PROTACs compound has been approved for treatment yet.

However, despite this uncertainty, the therapeutic potential of PROTACs for the treatment of diseases, especially those driven by yet “un-druggable” targets, is widely recognized in the art. The TPD approach in general is considered an important platform and implemented in the pipeline by most pharmaceutical companies. Although the high interest in this treatment, current limitations in the development of new TPDs include the drawback of comparatively screening slow methods, e.g., immunoblot, reporter gene assays, which are not capable of high throughput, and which have to be optimized anew for each target.

Therefore, there still exists an urgent need in the growing field of TPD drug development for approached suited for high-throughput screening and preferably involving quantitative measurement techniques, that can be applied to basically any target, including proteins that are only present in small amounts, and in vitro as well as in vivo.

Mass spectrometry constitutes such a measurement technique suitable on one hand for high-throughput measurements while enabling on the other hand quantitative measurements and universality regarding the analyzed problem, e.g., different candidate substances with different molecular downstream effects. In addition, mass spectrometry has been used in the past successfully for determining large-scale protein-protein interactions (interactomics), protein complex compositions and the analysis of whole signaling pathways. Several research groups have developed specific mass spectrometry approaches that facilitate the analysis of interactomes, which can also be applied to transient interactions, such as biotinylation-dependent proximity labelling.

BioID (Roux et al., 2012) is a method combining proximity-based biotinylation of proteins with mass spectrometry analysis, which can be employed in a cellular context in vitro and in vivo. In the BioID work-flow the promiscuous E. coli biotin ligase BirA* (BirA R118G) is fused in-frame to the protein of interest. This fusion protein enables biotin-labelling of proteins that come into the proximity of the protein of interest, as they are also entering the labelling-range of the promiscuous BirA* biotin ligase. The promiscuous biotin ligase activates biotin molecules, but only possesses a mild affinity for biotin itself, wherefore activated biotin simply diffuses away from the ligase-moiety of the fusion protein and reacts with nearby proteins. Subsequently, biotinylated proteins are enriched, even under harsh conditions, and analyzed by mass spectrometry. Some prior art methods, such as Jamilloux, Y. et al., 2018 (J. Biol. Chem., 293 (32) 12563-12575), applied a BioID proximity assay using a fusion protein of caspase1 and the biotin ligase BirA(R118G) to analyze protein degradation in the context of inflammation.

However, the BioID method using BirA* biotin ligase has the following shortcomings for analyzing protease or proteasomal interactions. First BirA* lacks a high enzymatic activity, such that the biotinylation of a sufficient amount of interaction partners of an enzyme or enzyme complex, such as the proteasome or a protease, would require at least 24 hours. Faster, more active variants of BirA* have been developed, e.g., TurboID, miniTurbo, that reach sufficient level of biotinylation in a time frame of 10 minutes to 2 hours. Alternative enzymes based on peroxidases, e.g., APEX and APEX2, can achieve efficient biotinylation in ~1 minute, but require treatment with hydrogen peroxide that can induce artifacts and is not compatible with in vivo applications.

Recently the BioID workflow has been used with the faster biotin ligase AirID by Yamanaka et al. 2022 (Nat. Commun. 13:183, 1-17). Yamanaka et al. used a fusion protein of AirID and the E3 ubiquitin ligase CRBN to analyze the protein interactions of CRBN, e.g., in the context of CRBN-targeting drugs used to treat hematological cancers.

However, even though some prior art methods analyzed the interactions of ubiquitin ligases or proteases (such as caspases) using variants of the BioID method, these prior art approaches are still not optimal for analyzing the interactions or targets of the proteasome itself. One reason is that even the activity of these faster, more active variants of BirA*, such as, e.g., TurboID, miniTurbo or AirID, would not have been expected to effectively label the highly transient and very brief interactions of the protease. For example, most substrates of proteases or the proteasome that are typically processed on a time scale of seconds. In addition, the final products of proteolytic degradation are oligopeptides that are sub-optimal for mass spectrometry identification due to their short length and heterogenous termini. Hence, it cannot be expected that said prior art approaches could be applied to the proteasome, as these shortcomings would lead to the detection of only an incomplete overview of the interactome and/or substrates of the proteasome or components thereof.

The ability of state-of-the-art approaches to detect selective recruitment of proteins to proteases, or protease complexes, such as the proteasome or components thereof, induced by small molecules has not been demonstrated. And no state-of-the-art approach has yet been shown to work in mouse models.

In light of these shortcomings of the prior art there remains a significant need to provide improved means to identify novel factors or chemicals that influence the recruitment of interaction partners and/or substrates to enzymes or enzyme complexes, such as the proteasome or components thereof, in vitro and in vivo.

### SUMMARY OF THE INVENTION

In view of the problems and shortcoming of the prior art, one problem to be solved by the present invention is the provision of improved or alternative means to identify interaction partners and/or substrates of enzymes or enzyme complexes, such as proteases or the proteasome, by reducing assay-related artefacts and improving detection sensitivity, which can be used in vitro and in vivo, for example to analyze the effects of compounds, e.g., pharmaceuticals, on said interactions and/or substrates of said enzymes or enzyme complexes.

The abovementioned problem is solved by the features of the independent claims. Preferred embodiments of the present invention are provided by the dependent claims.

The invention therefore relates in one aspect to a method of detecting an interaction or proximity between a fusion protein and a target protein, comprising
a. providing a fusion protein and/or a recombinant nucleic acid molecule encoding said fusion protein, said fusion protein comprising an enzyme or enzyme complex member fused to a promiscuous biotin ligase,
b. inhibiting the enzyme function of the fusion protein or the enzyme complex,
c. biotinylating a target protein that interacts with and/or is in proximity to the fusion protein,
d. enriching the biotinylated target protein, and
e. identifying the isolated target protein using (quantitative) mass spectrometry (MS).

In one embodiment, the invention relates to a method of detecting an interaction or proximity between a fusion protein and a target protein, comprising
a. providing a fusion protein and/or a recombinant nucleic acid molecule encoding said fusion protein, said fusion protein comprising a proteasome complex protein fused to a promiscuous biotin ligase,
b. inhibiting the enzyme function of the fusion protein or the enzyme complex,
c. biotinylating a target protein that interacts with and/or is in proximity to the fusion protein,
d. enriching the biotinylated target protein, and
e. identifying the isolated target protein using (quantitative) mass spectrometry (MS).

In some embodiments the present invention provides an improved method for quantitative mapping of interacting proteins (interactomes) and/or enzyme substrates for use in in vitro and in vivo studies. In embodiments the present method allows detection of both endogenous enzyme substrates and substrates delivered to an enzyme complex or machinery. Embodiments of the present method are, for example, suited to analyse the effects of novel therapeutic approaches and agents targeting a certain enzyme or enzyme complex in vitro and in vivo.

The proteins interacting with the enzyme or enzyme complex of interest in a particular cell state, tissue or upon a certain drug treatment may provide important information and open up new therapeutic opportunities.

As one of the challenges of analysing enzyme-protein interactions and enzyme-substrate interactions, e.g., interactions of the proteasome or of other proteases, are especially difficult, as such interactions are often brief and transient, and/or combined with fast degradation of enzyme substrates, e.g., in case of the example of the proteasome or protease substrates marked for digestion or degradation.

The inventors have developed a strategy based on the method according to the present invention for labelling enzymes or enzyme complexes (e.g., protein complexes comprising members with enzyme function) with promiscuous biotin ligases according monitor the interactome of said enzymes or enzyme complexes in vitro and in vivo.

In view of the very brief and highly transient interactions of proteins and substrates with the proteasome, it was entirely surprising that an approach based on the BioID-method may be used to analyze interactions of fusion proteins comprising biotin ligases and proteins (members) of the proteasome. Most substrates of the proteasome are typically processed on a time scale of seconds. In addition, the final products of proteasomal proteolytic degradation are oligopeptides that are sub-optimal for mass spectrometry identification due to their short length and heterogenous termini. Nevertheless, the present approach surprisingly enables the analysis of proteins interacting with and/or being processed by the proteasome.

For this purpose, in some embodiments a promiscuous biotin ligase (in embodiments BirA* (BirA(R118G)), BioID, BioID2, BASU, AirID, microID, microID2, ultraID, TurboID, miniTurbo, APEX or APEX2) is incorporated into or appended to an enzyme or partially or fully assembled enzyme complex by fusion with the enzyme itself or with a complex member subunit, without negatively affecting the enzyme or enzyme complex activity and its biological function. In preferred embodiments promiscuous biotin ligases label in situ, i.e., in the cellular context, all proteins that are within their proximity range (e.g., spanning several nm).

In specific embodiments the interactions of fully or at least partially assembled enzyme complexes can be analyzed according to the invention, wherein a split promiscuous biotin ligase is used, wherein one part of the biotin ligase is fused to one part of an enzyme complex and the other part of the biotin ligase is fused to another part of an enzyme complex, such that in a fully or at least partially assembled enzyme complex the parts of the split promiscuous biotin ligase will form a functional biotin ligase enzyme.

In embodiments where the enzyme complex or enzyme is the proteasome, or a protein or part or subunit thereof, it would have been very difficult to predict from the approaches disclosed in the prior art at which location, e.g., domain or protein of the proteasome a biotin ligase could be fused, such that the functional structure of the proteasome or subunit/part thereof is still preserved and the interaction with regular partners is possible. Moreover, depending on the interactions to be analyzed, the fusion localisation of the biotin ligase could potentially change, such that for some interaction partners and/or contexts the fusion would have to be performed at a different site, domain or protein member of the proteasome. For example, in some embodiments a preferred fusion site of the enzyme complex for interaction studies might be different, e.g., from the fusion site preferred for substrate detection experiments.

To increase the efficiency of substrate/target biotinylation during the present method in preferred embodiments biotin or derivates thereof are supplemented additionally for a certain period of time. In some embodiments supplementation of biotin or derivates thereof may be performed before the biotinylation step, or during the biotinylation step, or before and during the biotinylation step.

In embodiments the method according to the invention comprises additionally supplementing biotin or derivates thereof before the biotinylation step c. of the method according to the invention.

Subsequently, biotinylated target proteins are preferably isolated, for example, by a method known in the art (Mackmull et al. 2017) using (strept)avidin beads, are then preferably digested with proteases, and identified by mass spectrometry, preferably quantitative LC-MS/MS.

As can be derived from the Examples herein, specific improvements and advantageous adaptions to prior art protocols surprisingly significantly improved the background-to-signal ratio. In addition, in preferred embodiments DIA (Data Independent Acquisition) mass spectrometry is implemented for label-free analyses. In some embodiments, by finally using a particularly fast biotin ligase, such as e.g., miniTurbo, in combination with an inhibitor of the enzyme function of the enzyme itself or the enzyme function of the enzyme complex, the inventors surprisingly found, that it is possible to identify novel interaction partners of an enzyme or enzyme complex of interest, in addition to known ones. In embodiments endogenous and small molecule-induced enzyme or enzyme complex substrates (interactors) can be analyzed. In addition, the mouse model according to the invention allows corresponding analyses in vivo. Embodiments of this mouse line, constitutively expresses a transactivator, e.g., rTA3, thereby enabling doxycycline-inducible expression of the enzyme or an enzyme complex subunit fused to a particularly fast biotin ligase, e.g., miniTurbo, in all tissues or in some tissues of the animal.

Embodiments of the present method as well as the present cell and animal model, e.g., mouse model, are particularly suited as automated high throughput screening platforms that can be used to identify molecules targeting an enzyme or enzyme complex of interest and/or the recruitment of interactors and substrates to them. Furthermore, the different aspects of the present invention, such as the method, the cell and/or the mouse model are also suitable for the validation of molecules or pharmaceuticals (candidate substance(s)) in vitro and in vivo.

Hence, in embodiments the method according to the invention comprises additionally treating the cell in which said fusion protein is expressed with at least one candidate substance.

In embodiments of the method according to the invention the at least one candidate substance is administered prior to biotin supplementation. In embodiments the candidate substance is administered at least 1 minute, at least 2, 3, 4, 5, 10, 15, 20, 25, 30, 35, 40, 45, 50, 55, 60, 70, 80, 90, 100, 120, 150, 180, 210, 240, 270, 300 minutes, or at least 1, 2, 3, 4, 5, 6, 7, 8, 9, 10, 12, 24, 36, 48, 96, 168, 336 hours prior to biotin supplementation.

In embodiments the candidate substance is administered at least 1 minute, at least 2, 3, 4, 5, 10, 15, 20, 25, 30, 35, 40, 45, 50, 55, 60, 70, 80, 90, 100, 120, 150, 180, 210, 240, 270, 300 minutes, or at least 1, 2, 3, 4, 5, 6, 7, 8, 9, 10, 12, 24, 36, 48, 96, 168, 336 hours after biotin supplementation, or for 1 to 336, 1 to 168, 1 to 48, 1 to 24, 1 to 12, 1 to 6, 1 to 4 hours during biotin supplementation, or at least for 1, 2, 3, 4, 5, 10, 15, 20, 25, 30, 35, 40, 45, 50, 55, 60, 70, 80, 90, 100, 120, 150, 180, 210, 240, 270, 300 minutes, or at least for 1, 2, 3, 4, 5, 6, 7, 8, 9, 10, 12, 24, 36, 48, 96, 168, 336 hours, or at least for 1-30 days, for 1 to 14 days, for 1 to 7 days during biotin supplementation.

Thus, the combination of proximity labelling, and mass spectrometry provides a quantitative read out of recruitment of target protein(s) to enzymes or enzyme complexes of interest. Since labelling occurs in the cellular context, artifacts due, e.g., to cell lysis are avoided. The preferred use of shot gun mass spectrometry enables unbiased detection of target recruitment to enzymes or enzyme complexes and therefore enables in embodiments to reveal off-target effects of, e.g., an analyzed candidate compound. The approach enables in embodiments to detect targets across a broad range of protein abundance, as demonstrated by the detection of low abundant enzyme substrates such as transcription factors, as shown for one embodiment for the proteasome in Example 1 and FIG. 6.

In preferred embodiments streptavidin contamination can be reduced by chemical modification, such as, e.g., acetylation, of the lysine and/or arginine residues of streptavidin and/or preferably by additionally using the protease LysC instead of trypsin for on-bead digestion during sample preparation for mass spectrometry analysis, thereby significantly improving the signal:background ratio.

In some specific embodiments the substrates of the enzyme or enzyme complex are enzymatically processed by the fusion protein or the enzyme-subunit of the enzyme complex. In preferred embodiments the enzyme function is inhibited by an inhibitor compound at least during the proximity-based labelling process and preferably until sample analysis or enrichment of the target proteins, such that substrates are not processed by the fusion protein or the enzyme-subunit of the enzyme complex and can be enriched and identified after proximity-biotinylation.

In embodiments of the method according to the invention the enzyme inhibitor is a protease inhibitor compound, preferably a small molecule protease inhibitor, more preferably MG132 or derivate thereof.

In embodiments of the method according to the invention the enzyme inhibitor or protease inhibitor compound is administered at least 1 to 4 hours prior to/or for at least 1 to 24 hours or 1 to 30 days during biotin supplementation.

In embodiments the time point and duration of the candidate substance and/or inhibitor administration and/or biotin supplementation is dependent on the system or model used and/or the enzyme or enzyme complex and/or the candidate substance analyzed.

In embodiments the enzyme inhibitor compound or protease inhibitor compound is administered at least 1 minute, at least 2, 3, 4, 5, 10, 15, 20, 25, 30, 35, 40, 45, 50, 55, 60, 70, 80, 90, 100, 120, 150, 180, 210, 240, 270, 300 minutes, or at least 1, 2, 3, 4, 5, 6, 7, 8, 9, 10, 12, 24, 36, 48, 96, 168, 336 hours prior to biotin supplementation, or for 1 to 336, 1 to 168, 1 to 48, 1 to 24, 1 to 12, 1 to 6, 1 to 4 hours during biotin supplementation, or at least for 1, 2, 3, 4, 5, 10, 15, 20, 25, 30, 35, 40, 45, 50, 55, 60, 70, 80, 90, 100, 120, 150, 180, 210, 240, 270, 300 minutes, or at least for 1, 2, 3, 4, 5, 6, 7, 8, 9, 10, 12, 24, 36, 48, 96, 168, 336 hours, or at least for 1-30 days, for 1 to 14 days, for 1 to 7 days during biotin supplementation.

Another preferred feature of the present invention is the combination of a particularly fast biotin ligase, such as e.g., TurboID or miniTurbo, with an inhibitor of the enzymes or enzyme complexes enzyme function. Thereby especially enzyme or enzyme complex substrates are not processed and can be enriched and identified after proximity-biotinylation.

In embodiments of the invention, where a cell culture or in vitro reaction is used the inhibitor compound and/or the candidate substance may be administered by adding the inhibitor compound and/or the candidate substance to the culture medium. In case of in vitro cell-free assays the inhibitor compound and/or the candidate substance may be administered into the reaction solution or reaction buffer. In embodiments where an animal according to the invention is used, the inhibitor compound and/or the candidate substance may be administered or supplied, for example, through the drinking water, the chow or food, oral gavage, transdermal and/or injection.

Hence, in embodiments of the method according to the invention the promiscuous biotin ligase is a E. coli or B. subtilis BirA biotin ligase or variant thereof in its integral or split form.

In embodiments of the method according to the invention the promiscuous biotin ligase is selected from the group consisting of BirA* (BirA(R118G)), BioID, BioID2, BASU, AirID, microID, microID2, ultraID, TurboID, miniTurbo, APEX and APEX2.

In embodiments of the method according to the invention the promiscuous biotin ligase is miniTurbo.

In embodiments of the method according to the invention the promiscuous biotin ligase is BirA*.

In embodiments the promiscuous biotin ligase transfers biotin or derivates thereof to substrates and interactors that are in close proximity of the fusion protein or directly interact with and/or bind to it. In preferred embodiments the close proximity is a distance between 0.01 nm and 100 nm, preferably between 0.001 nm and 50 nm or even more preferably between 0.001 and 10 nm. In preferred embodiments the close proximity is a distance of less than 20 nm, more preferably less than 10 nm. In some embodiments the close proximity is a distance of less than 0.001, 0.01, 0.1, 1, 2, 3, 4, 5, 6, 7, 8, 9, 10, 11, 12, 13, 14, 15, 16, 17, 18, 19, 20, 21, 22, 23, 24, 25, 50 or 100 nm.

In preferred embodiments a particularly fast promiscuous biotin ligase, e.g., miniTurbo or TurboID, comprising a high enzymatic activity to activate biotin or derivates thereof, is used. In embodiments the working speed (activation of a certain amount of biotin) of the biotin ligase is faster than those of wild type biotin ligases, such as BirA, or than the promiscuous biotin ligase BirA*. A faster working speed (higher activity) of a biotin ligase means in this context that more biotin molecules or derivatives thereof are activated in the same time, when compared to another biotin ligase.

Hence, in preferred embodiments a fast-working biotin ligase biotinylates a large amount/high number of interactors and/or substrates in less than 24 hours. Accordingly, in preferred embodiments a fast-working biotin ligase requires less time to activate the same amount/number of biotin or derivates thereof as BirA or BirA*. In other words, in preferred embodiments a fast-working biotin ligase activates the same amount/number of biotin or derivates thereof as BirA or BirA* would process in 24 hours in a time shorter than 24 hours, preferably shorter than 12 hours, more preferably in less than 1 hour. Preferably the fast-working biotin ligase activates at least two times as many biotin molecules as BirA or BirA* would activate in the same time, preferably even three, four, five, ten or 20 times as many biotin molecules.

One of the advantageous effects of the usage of a particularly fast working promiscuous biotin ligase is that a large amount/high numbers of interactors and/or substrates of the enzyme or enzyme complex can be biotinylated in a very short time. In embodiments where the enzyme or enzyme complex has a function essential for the survival or viability of the cell, the inhibition of the enzyme or enzyme complex in step b. would entail detrimental effects on the survival or metabolism of the cell. Accordingly, a fast biotin ligase requites only a short time, e.g. less than 24 hours, preferably less than 6 hours, more preferably only 1 hour or even less to biotinylate all interactors and/or substrates of an enzyme or enzyme complex, such that the inhibition of the enzyme or enzyme complex is reduced to a time span that has no negative effect on the cell or induces no or few stress signals of the, such that stress-induced artifacts in interactions can be reduced. Thereby the present method facilitates, especially through the use of a fast biotin ligase, such as BASU, AirID, microID, microID2, ultraID, TurboID, miniTurbo, APEX and APEX2 the reduction of interaction and/or substrate “artefacts” that are induced solely by the inhibition of the enzyme or enzyme complex and its effects on the cell, namely artefacts induced by the assay itself.

In embodiments the naturally available amount of biotin or derivates thereof may not be sufficient to ensure reliable biotinylation of each protein in the proximity of a fusion protein. Hence in embodiments, to avoid any shortage in biotin or derives thereof for labelling of target proteins (resulting, e.g., in potential reduced labelling and false-negative detection), biotin or derivates thereof may be supplied additionally. The means and ways of administration may be dependent and specific for the method and system or model (in vitro or in vivo) according to the invention that is used, e.g., a reaction solution, a cell or an animal. For example, in embodiments, a cell culture or in vitro reaction may be supplemented with biotin or derivates thereof by adding biotin or derivates thereof to the culture medium. In case of in vitro cell-free assays the biotin or derivates thereof may be supplemented to the reaction solution or reaction buffer. In embodiments where an animal according to the invention is used, the biotin or derivates thereof may be supplied, for example, through the drinking water, the chow (food), oral gavage, transdermal or injection.

In embodiments the present method surprisingly enables the identification of interaction partners/interactors and/or substrates of the enzyme or enzyme complex in a high-throughput screening manner, which may be for the development of new drugs or screening of candidate compounds that influence such protein-protein interactions and/or enzyme-substrate interactions.

In embodiments of the method according to the invention the fusion protein comprises a proteasome complex protein fused to the biotin ligase.

In embodiments the fusion protein comprises a proteasome complex protein or part thereof fused to a biotin ligase or part thereof.

In embodiments of the method according to the invention the proteasome complex protein is a part of 11S proteasome activators (consisting of PA26, PA28α/REGα, PA28β/REGβ, PA28γ/REGγ, or any combination of), PA200/Blm10 proteasome activator, PAN protein complex, ARC/Mpa protein complex, 19S, 20S, 26S, or 30S proteasome particle.

In embodiments of the method according to the invention the proteasome complex protein is selected from the group consisting of PSMA1-8, PSMB1-11, PSMC1-6, PSMD1-14, PSME1-4, PSMF1, PAMD12, ADRM1, USP14/Ubp6, UCHL5/UCH37, UBE3C/KIAA10/Hul5, SHFM1/DSS1, or any combination thereof.

In embodiments the proteasome complex protein is selected from the group consisting of PSMA1-8, PSMB1-11, PSMC1-6, PSMD1-14, PSME1-4, PSMF1, or any combination thereof.

In embodiments the proteasome complex protein is selected from the group consisting of PSMA1-8, PSMC1-6 and PSMD1-14 or any combination thereof.

In preferred embodiments the proteasome complex protein is selected from the group consisting of PSMA4, PSMC2 and PSMD3 or any combination thereof.

In embodiments of the method according to the invention the target protein to be identified is a substrate or interaction partner of the proteasome.

In a specific embodiment a split promiscuous biotin ligase is used, wherein one part of the biotin ligase is fused to a 19S regulatory particle of the proteasome, and the other part of the biotin ligase is fused to a 20S core particle of the proteasome, such that in a fully assembled proteasomal complex the parts of the split promiscuous biotin ligase will form a functional biotin ligase enzyme.

In embodiments the fusion protein comprises a biotin ligase enzyme or part thereof, wherein in a fully assembled enzyme complex, e.g. a proteasomal complex, the biotin ligase constitutes or is assembled to a functional biotin ligase enzyme.

In embodiments the fusion protein and/or recombinant nucleic acid molecule encoding said fusion protein comprises or consists of and/or encodes an amino acid sequence according to SEQ ID NO. 1 (MSRRYDSRTTIFSPEGRLYQVEYAMEAIGHAGTCLGILANDGVLLAAERRNIHKLLDEV FFSEKIYKLNEDMACSVAGITSDANVLTNELRLIAQRYLLQYQEPIPCEQLVTALCDIKQAYTQFG GKRPFGVSLLYIGWDKHYGFQLYQSDPSGNYGGWKATCIGNNSAAAVSMLKQDYKEGEMTLKS ALALAIKVLNKTMDVSKLSAEKVEIATLTRENGKTVIRVLKQKEVEQLIKKHEEEEAKAEREKKEKE QKEKDKYPTFLYKVVDSRMKDNTVPLKLIALLANGEFHSGEQLGETLGMSRAAINKHIQTLRDWG VDVFTVPGKGYSLPEPIQLLNAKQILGQLDGGSVAVLPVIDSTNQYLLDRIGELKSGDACIAEYQQ AGRGGRGRKWFSPFGANLYLSMFWRLEQGPAAAIGLSLVIGIVMAEVLRKLGADKVRVKWPND LYLQDRKLAGILVELTGKTGDAAQIVIGAGINMAMRRVEESVVNQGWITLQEAGINLDRNTLAAML IRELRAALELFEQEGLAPYLSRWEKLDNFINRPVKLIIGDKEIFGISRGIDKQGALLLEQDGIIKPWM GGEISLRSAEKGGSGPGGGAPDYKDDDDK) or an amino acid sequence with a sequence identity of least 60%, of least 70%, preferably of at least 80%, more preferably of at least 90% to SEQ ID NO 1. In embodiments said fusion protein and/or a recombinant nucleic acid molecule encoding said fusion protein according to SEQ ID NO. 1 or a sequence with least 60% sequence identity thereto, comprises a sequence of PSMA4, or a fragment thereof, fused to a sequence of BirA, or a fragment thereof.

In embodiments the fusion protein and/or recombinant nucleic acid molecule encoding said fusion protein comprises or consists of and/or encodes an amino acid sequence according to SEQ ID NO. 2 (MPDYLGADQRKTKEDEKDDKPIRALDEGDIALLKTYGQSTYSRQIKQVEDDIQ QLLKKINELTGIKESDTGLAPPALWDLAADKQTLQSEQPLQVARCTKIINADSEDPKYIINVKQFAK FVVDLSDQVAPTDIEEGMRVGVDRNKYQIHIPLPPKIDPTVTMMQVEEKPDVTYSDVGGCKEQIE KLREVVETPLLHPERFVNLGIEPPKGVLLFGPPGTGKTLCARAVANRTDACFIRVIGSELVQKYVG EGARMVRELFEMARTKKACLIFFDEIDAIGGARFDDGAGGDNEVQRTMLELINQLDGFDPRGNIK VLMATNRPDTLDPALMRPGRLDRKIEFSLPDLEGRTHIFKIHARSMSVERDIRFELLARLCPNSTG AEIRSVCTEAGMFAIRARRKIATEKDFLEAVNKVIKSYAKFSATPRYMTYNCPTFLYKVVDSRMKD NTVPLKLIALLANGEFHSGEQLGETLGMSRAAINKHIQTLRDWGVDVFTVPGKGYSLPEPIQLLNA KQILGQLDGGSVAVLPVIDSTNQYLLDRIGELKSGDACIAEYQQAGRGGRGRKWFSPFGANLYL SMFWRLEQGPAAAIGLSLVIGIVMAEVLRKLGADKVRVKWPNDLYLQDRKLAGILVELTGKTGDA AQIVIGAGINMAMRRVEESVVNQGWITLQEAGINLDRNTLAAMLIRELRAALELFEQEGLAPYLSR WEKLDNFINRPVKLIIGDKEIFGISRGIDKQGALLLEQDGIIKPWMGGEISLRSAEKGGSGPGGGA PDYKDDDDK) or an amino acid sequence with a sequence identity of least 60%, of least 70%, preferably of at least 80%, more preferably of at least 90% to SEQ ID NO 2. In embodiments said fusion protein and/or a recombinant nucleic acid molecule encoding said fusion protein according to SEQ ID NO. 2, or a sequence with least 60% sequence identity thereto, comprises a sequence of PSMC2, or a fragment thereof, fused to a sequence of BirA, or a fragment thereof.

In embodiments the fusion protein and/or recombinant nucleic acid molecule encoding said fusion protein comprises or consists of and/or encodes an amino acid sequence according to SEQ ID NO. 3 (MKDNTVPLKLIALLANGEFHSGEQLGETLGMSRAAINKHIQTLRDWGVDVFTVPGKG YSLPEPIQLLNAKQILGQLDGGSVAVLPVIDSTNQYLLDRIGELKSGDACIAEYQQAGRGGRGRK WFSPFGANLYLSMFWRLEQGPAAAIGLSLVIGIVMAEVLRKLGADKVRVKWPNDLYLQDRKLAGI LVELTGKTGDAAQIVIGAGINMAMRRVEESVVNQGWITLQEAGINLDRNTLAAMLIRELRAALELF EQEGLAPYLSRWEKLDNFINRPVKLIIGDKEIFGISRGIDKQGALLLEQDGIIKPWMGGEISLRSAE KGGSGPGGGAPDYKDDDDKLGAPTSLYKKVGMKQEGSARRRGADKAKPPPGGGEQEPPPPP APQDVEMKEEAATGGGSTGEADGKTAAAAAEHSQRELDTVTLEDIKEHVKQLEKAVSGKEPRF VLRALRMLPSTSRRLNHYVLYKAVQGFFTSNNATRDFLLPFLEEPMDTEADLQFRPRTGKAAST PLLPEVEAYLQLLVVIFMMNSKRYKEAQKISDDLMQKISTQNRRALDLVAAKCYYYHARVYEFLD KLDVVRSFLHARLRTATLRHDADGQATLLNLLLRNYLHYSLYDQAEKLVSKSVFPEQANNNEWA RYLYYTGRIKAIQLEYSEARRTMTNALRKAPQHTAVGFKQTVHKLLIVVELLLGEIPDRLQFRQPS LKRSLMPYFLLTQAVRTGNLAKFNQVLDQFGEKFQADGTYTLIIRLRHNVIKTGVRMISLSYSRISL ADIAQKLQLDSPEDAEFIVAKAIRDGVIEASINHEKGYVQSKEMIDIYSTREPQLAFHQRISFCLDIH NMSVKAMRFPPKSYNKDLESAEERREREQQDLEFAKEMAEDDDDSFP) or an amino acid sequence with a sequence identity of least 60%, of least 70%, preferably of at least 80%, more preferably of at least 90% to SEQ ID NO 3. In embodiments said fusion protein and/or a recombinant nucleic acid molecule encoding said fusion protein according to SEQ ID NO. 3, or a sequence with least 60% sequence identity thereto, comprises a sequence of BirA, or a fragment thereof, fused to a sequence of PSMD3, or a fragment thereof.

In embodiments the fusion protein and/or recombinant nucleic acid molecule encoding said fusion protein comprises or consists of and/or encodes an amino acid sequence according to SEQ ID NO. 4 (MSRRYDSRTTIFSPEGRLYQVEYAMEAIGHAGTCLGILANDGVLLAAERRNIHKLLDEVF FSEKIYKLNEDMACSVAGITSDANVLTNELRLIAQRYLLQYQEPIPCEQLVTALCDIKQAYTQFGG KRPFGVSLLYIGWDKHYGFQLYQSDPSGNYGGWKATCIGNNSAAAVSMLKQDYKEGEMTLKSA LALAIKVLNKTMDVSKLSAEKVEIATLTRENGKTVIRVLKQKEVEQLIKKHEEEEAKAEREKKEKEQ KEKDKYPTFLYKVVDSRMASIPLLNAKQILGQLDGGSVAVLPVVDSTNQYLLDRIGELKSGDACIA EYQQAGRGSRGRKWFSPFGANLYLSMFWRLKRGPAAIGLGPVIGIVMAEALRKLGADKVRVKW PNDLYLQDRKLAGILVELAGITGDAAQIVIGAGINVAMRRVEESVVNQGWITLQEAGINLDRNTLA AMLIRELRAALELFEQEGLAPYLSRWEKLDNFINRPVKLIIGDKEIFGISRGIDKQGALLLEQDGVIK PWMGGEISLRSAEKLQDDYKDDDDK) or an amino acid sequence with a sequence identity of least 60%, of least 70%, preferably of at least 80%, more preferably of at least 90% to SEQ ID NO 4. In embodiments said fusion protein and/or a recombinant nucleic acid molecule encoding said fusion protein according to SEQ ID NO. 4, or a sequence with least 60% sequence identity thereto, comprises a sequence of PSMA4, or a fragment thereof, fused to a sequence of miniTurboID, or a fragment thereof.

In preferred embodiments the step d. of enriching the biotinylated target protein comprises the enrichment of biotinylated target protein with a mobile or stationary solid phase.

In embodiments the step d. of enriching the biotinylated target protein comprises the enrichment of biotinylated target protein with a mobile or stationary solid phase comprising streptavidin, avidin, NeutrAvidin and/or an antibody directed against biotin or derivates thereof.

In embodiments said mobile or stationary solid phase is a bead, a surface, a column or a resin. In some embodiments the mobile or stationary solid phase is a bead comprising streptavidin, avidin, NeutrAvidin and/or an antibody directed against biotin or derivates thereof.

In certain embodiments a side effect of the enrichment of biotinylated target proteins by streptavidin-coupled solid phases can be a streptavidin-related contamination, namely increased background noise during subsequent mass spectrometry measurements. This effect arises from the digestion of streptavidin by the proteases LysC or trypsin during the “on-bead” digestion step of sample preparation for MS analysis (namely the digestion of target proteins while still bound to the solid enrichment-phase). To avoid the digestion (proteolysis) of the streptavidin on the solid phases by trypsin and/or LysC, streptavidin can preferably be chemically modified, thereby rendering it resistant to proteolysis by LysC and/or trypsin.

Accordingly, in embodiments the lysine residues of streptavidin, used for enriching the biotinylated target protein, are acetylated. This feature results in a surprising reduction of streptavidin contamination. Hence, in embodiments streptavidin contamination is reduced by acetylation of the lysine residues of streptavidin used for enriching the biotinylated target protein.

Accordingly, in some preferred embodiments the mobile or stationary solid phase comprises streptavidin, wherein the lysine residues of the streptavidin are acetylated.

In other embodiments the same or a similar effect can be achieved by the chemical modification of lysine and arginine residues of streptavidin by demethylation and condensation.

In embodiments mass spectrometry analysis comprises digesting the enriched biotinylated target protein with the protease LysC instead of trypsin, preferably during on-bead digestion. The digestion of the enriched biotinylated target protein with the protease LysC instead of trypsin achieved the unexpected effect of significantly improving the signal:background ratio (up to >4 fold) during mass spectrometry measurements, e.g., as shown in FIG. 8B.

Hence, in some embodiments identifying the isolated target protein using (quantitative) mass spectrometry (MS) comprises digesting the enriched biotinylated target protein with the protease LysC, wherein the digestion of the enriched biotinylated target protein is an on-bead digestion. In other words, in preferred embodiments enriched biotinylated target proteins are digested with LysC during “on-bead” digestion in step e. of identifying the isolated target protein using (quantitative) mass spectrometry (MS).

The “on-bead” digestion step, namely the digestion (proteolysis) of (target) proteins that are bound to the solid enrichment-phase, is not necessarily limited to solid-phases that are or comprise “beads”, it can in other embodiments also be performed on other types of solid phases, such as stationary solid phases, for example, a column, a surface or a resin.

The on-bead digestion is preferably conducted before the mass spectrometry measurement of the enriched/isolated target proteins. The on-bead digestion is preferably a proteolysis by the enzyme(s) trypsin and/or LysC. In preferred embodiments the on-bead-digested target proteins are subsequently eluted from the solid phase and analyzed by mass spectrometry, preferably combined with liquid chromatography (LC) previous to mass spectrometry (LC-MS).

Hence, in embodiments the step e. of identifying the isolated target protein using (quantitative) mass spectrometry (MS) further comprises the separation or processing of the enriched target proteins by liquid chromatography before mass spectrometry measurements. In other words, step e. of identifying the isolated target protein using (quantitative) mass spectrometry (MS) comprises analysis with liquid chromatography-mass spectrometry (LC-MS), preferably after proteolytic (on-bead) digestion of the isolated target protein(s).

In preferred embodiments step d. of enriching the biotinylated target protein comprises the enrichment of biotinylated target protein with a mobile or stationary solid phase, and step e. of identifying the isolated target protein using (quantitative) mass spectrometry (MS) comprises:
i. on-bead digestion of the enriched target proteins, and/or
ii. elution of peptides derived from target proteins still bound to the solid phase, and/or
iii. separation of peptides from the enriched target proteins by liquid chromatography (LC), and
iv. analysis of the LC-separated peptides from target proteins by mass spectrometry.

In embodiments the fraction of the peptides, resulting from the on-bead digestion of the target proteins, that are not bound to the solid phase after the on-bead digestion are analyzed separately from the peptides, that are still bound to the solid phase (as they comprise at least one biotin bound to the solid phase) after on-bead digestion. Preferably peptides that are bound to the solid phase are acquired by elution from the solid phase, e.g., preferably by acetonitrile (ACN) and/or trifluoroacetic acid (TFA).

In preferred embodiments the mass spectrometry is a liquid chromatography mass spectrometry/mass spectrometry (LC-MS/MS). Herein, LC-MS/MS may also be termed tandem LC-MS.

In embodiment Data Independent Acquisition (DIA) is implemented for the analysis of the enriched biotinylated target proteins by mass spectrometry. The implementation of Data Independent Acquisition (DIA) for the analysis of biotinylated target protein surprisingly increased more than 2-fold the number of identified proteins and biotinylated peptides, as shown e.g., in FIG. 8C. In comparison to data-dependent acquisition (DDA), in which precursors are selected by intensity for fragmentation, in DIA, all precursors within specified mass ranges (so called precursor isolation windows) are fragmented.

In embodiments of the method according to the invention when step a. of the present method comprises a recombinant nucleic acid sequence encoding said fusion protein, expression of said nucleic acid molecule is under control of an inducible promoter.

The regulation of the expression of the fusion protein by an inducible promoter in some embodiments of the invention is of particular advantage, if the permanent expression of the fusion protein is not advantageous for the growth of a cell or survival or health of an animal described herein, or if the fusion protein should not be expressed in each cell or tissue or at all times.

Hence, tissue or time point-specific induction of the expression of the fusion protein can preferably be regulated through an inducible promotor in a desired fashion.

In embodiments of the method according to the invention the inducible promoter is controlled by administration of doxycycline or tetracycline, and the nucleic acid comprises a doxycycline/tetracycline-controlled Tet-Off and Tet-On gene expression system.

An example of the advantageous effects of this embodiment can be derived especially from the present Example 2.

In embodiments of the method according to the invention the biotinylated target protein occurs inside or outside a cell in which the fusion protein is expressed. In other words, in embodiments the biotinylated target protein is located inside or outside of a cell in which the fusion protein is expressed, wherein outside refers to a location on the cell's surface, or on and/or at least partially within the outer cell membrane.

In embodiments of the method according to the invention the fusion protein optionally further comprises a protein- or affinity-tag, such as a FLAG-, GFP-, Strep-, GST-, His-, CBP-, CBD-, MBP-, c-Myc-, Halo-, Protein G-, Protein A-, HA- or T7-tag.

The use of a protein or affinity tag in embodiments can have different advantages. One advantage is that the expression and/or localization and/or tissue specific expression of the fusion protein can be detected or tracked using either a fluorescent tag, such as GFP, or using a tag-specific labelled antibody, e.g., fluorescently labelled antibody, or by immunoblot using a tag-specific antibody.

In another aspect the present invention relates to a method of detecting an interaction or proximity between a fusion protein and a target protein according to the invention, comprising
i. providing a recombinant nucleic acid molecule encoding the fusion protein, said fusion protein comprising a proteasome complex protein fused to a promiscuous biotin ligase selected from the group comprising BirA*(BirA(R118G)), BioID, BioID2, BASU, AirID, microID, microID2, ultraID, TurboID, miniTurbo, APEX and APEX2
ii. expressing the fusion protein,
iii. inhibiting of protease function of the fusion protein or proteasome complex,
iv. supplementing biotin or derivates thereof,
v. biotinylating a target protein that is in proximity to and/or interacts with the fusion protein,
vi. enriching the biotinylated target protein,
vii. identifying the isolated target protein using quantitative mass spectrometry, preferably liquid chromatography mass spectrometry (LC/MS), more preferably LC-tandem MS (LC-MS/MS).

In another aspect the present invention relates to a nucleic acid molecule encoding a fusion protein comprising a proteasome complex protein fused to a promiscuous biotin ligase selected from the group consisting of BirA* (BirA(R118G)), BioID, BioID2, BASU, AirID, microID, microID2, ultraID, TurboID and miniTurbo.

In another aspect the present invention relates to a fusion protein comprising a proteasome complex protein fused to a promiscuous biotin ligase selected from the group consisting of BirA*(BirA(R118G)), BioID, BioID2, BASU, AirID, microID, microID2, ultraID, TurboID and miniTurbo.

In embodiments the fusion protein comprises a proteasome complex protein fused to the promiscuous biotin ligase miniTurbo.

In embodiments the fusion protein comprises a proteasome complex protein fused to the promiscuous biotin ligase BirA*.

In another aspect the present invention relates to a genetically modified cell comprising the nucleic acid according to the invention and/or expressing the fusion protein according to the invention.

In another aspect the present invention relates to a genetically modified mouse comprising the nucleic acid molecule according to the invention and/or expressing the fusion protein according to the invention in at least one tissue, wherein the nucleic acid sequence encoding the fusion protein is preferably under the control of a doxycycline/tetracycline-controlled Tet-Off and Tet-On gene expression system.

In another aspect the present invention relates to a genetically modified model organism, such as a rodent, mouse, rat, fly, nematode or fish, comprising the nucleic acid molecule according to the invention and/or expressing the fusion protein according to the invention in at least one tissue, wherein the nucleic acid sequence encoding the fusion protein is preferably under the control of a doxycycline/tetracycline-controlled Tet-Off and Tet-On gene expression system.

In one embodiment the animal model, e.g., mouse model, is designed to express a member of an enzyme complex, such as the 20S proteasome core particle PSMA4, fused to a promiscuous biotin ligase, such as miniTurbo, and a FLAG tag for the detection of the fusion protein. In this embodiment the fusion gene, e.g., PSMA4-miniTurbo, is inserted in a locus, e.g., the Col1a1 locus, downstream of a tetracycline responsive element (TRE) in a mouse embryonic stem cell line, e.g., D34. In embodiments, the embryonic stem cell line preferably carries a cassette encoding a transactivator, such as, e.g., the rTA3 transactivator, a fluorescent protein, e.g., GFP or mKate, on the Rosa 26 locus under the control of a CAG promoter. In embodiments in the cell line comprises a LoxP-stop-LoxP cassette between the CAG promoter and the transactivator, e.g., rTA3, and the fluorescent protein gene, e.g., mKate, expressing cassette, enabling tissue-specific expression via crossing to specific CRE lines. The engineered cell line is then used to generate the animal model, e.g., a mouse line via blastocyst injection.

In embodiments, the engineered model animal, e.g., mouse, can then be crossed with a CMV-Cre line that expresses constitutively the CRE recombinase in all or in desired selected tissues.

After confirming successful excision of the lox-stop-lox cassette, the obtained model animal, e.g., mouse line, is back crossed to, e.g., C57BL6/J, to remove the CMV-Cre allele. The obtained model animal, e.g., mouse line, constitutively expresses transactivator, such as, e.g., the rTA3 transactivator, thereby enabling doxycycline inducible expression of the fusion gene construct in all or in the desired selected tissues.

A further aspect of the invention relates to a kit comprising the nucleic acid molecule as described herein, and one or more of:
i. means for cell-transfection, transduction, or infection or gene knock-in,
ii. doxycycline or tetracycline,
iii. biotin or derivate thereof, and/or
iv. a protease-inhibitor.

In another aspect the present invention relates to the use of the method according to the invention for determining an effect of a candidate substance on the interaction and/or proximity between a proteasomal complex protein and a target protein.

Each feature of the invention that is disclosed in the context of one aspect of the invention is herewith also disclosed in the context of the other inventive aspects disclosed herein.

Accordingly, embodiments and features of the invention described with respect to the method disclosed herein, are considered to be disclosed with respect to each and every other aspect of the disclosure, such that features characterizing one embodiment of the present method, cell line, mouse model or fusion protein, may be employed to characterize another embodiment of the method, cell line, mouse model or fusion protein, and vice-versa. The various aspects of the invention are unified by, benefit from, are based on and/or are linked by the common and surprising finding of the unexpected advantageous effects of the present method to identify interaction partners of an enzyme or enzyme complex by creating a fusion protein of said enzyme or enzyme complex or complex member thereof with a promiscuous biotin ligase, followed by detecting biotinylated interactors through an improved mass spectrometry approach.

### DETAILED DESCRIPTION OF THE INVENTION

The present invention relates in preferred embodiments to a method for detecting an interaction or proximity between a fusion protein and a target protein, wherein a fusion protein and/or a recombinant nucleic acid molecule encoding said fusion protein, is provided, wherein the fusion protein comprises an enzyme or enzyme complex member that is fused to a promiscuous biotin ligase. Subsequently, the enzyme function of the fusion protein or the enzyme complex is inhibited, followed by biotinylating a target protein that interacts with and/or is in proximity to the fusion protein, and finally enriching the biotinylated target protein, and identifying the isolated target protein using mass spectrometry.

In the context of the present invention the term “subject” refers to an individual, a patient, a human, an animal, a model animal, a mammal, a vertebrate, or a cell or cell culture, preferably a (model) animal or a human. In preferred embodiments the subject is a human, or an animal.

Herein a “sample” may be taken from a subject, a patient, a cell culture of patient cells or cell lines, an animal, or a cell culture of animal cells or cell lines of a biopsy, a blood sample, a tissue sample, or an environmental sample. Basically, any kind of sample that is suspected to contain biochemical information of interest. As used herein, the term “sample” is a biological sample that is obtained or isolated from the subject, Sample as used herein may, e.g., refer to a sample of bodily fluid, tissue or surface (e.g., mucosal swap sample) obtained for the purpose of diagnosis, prognosis, or evaluation of a subject of interest. In case of a liquid sample, or a liquid biopsy the sample may be in embodiments a sample of a bodily fluid, such as blood, serum, plasma, cerebrospinal fluid, urine, saliva, sputum, pleural effusions, a cellular extract, and the like. In further embodiment the sample may be a solid sample, such as a biopsy, a tissue sample, a cell culture sample, cells, a tissue sample, a tissue biopsy, a stool sample or a swap-derived sample.

“Recombinant nucleic acids” or recombinant DNA (rDNA) molecules are DNA molecules formed by laboratory methods of genetic recombination (such as molecular cloning) that bring together genetic material from multiple sources, creating sequences that would not otherwise be found in the genome. “Recombinant DNA” is the general name for a piece of DNA that has been created by combining at least two fragments from two different sources.

A “fusion protein” or chimeric protein is a protein created through the joining of two or more genes that originally code for separate proteins. Translation of this fusion gene results in a single or multiple polypeptides with functional properties derived from each of the original proteins. A recombinant fusion protein is usually created artificially using molecular cloning techniques. Herein preferably a fusion protein retains the functional and structural properties of the proteins fused to each other, such that in preferred embodiments the biotin-ligase-function of the biotin ligase enzyme that is fused to an enzyme or a member of an enzyme complex is retained, as well as the enzyme function of the single enzyme or enzyme complex. Preferably the fusion protein comprises a biotin ligase fused to an enzyme or to member of an enzyme complex. Said enzyme or member of an enzyme complex or the enzyme complex itself may herein also be referred to as the “bait”. This is, as the bait preferably attracts its natural interaction partners to the fusion protein, such that the promiscuous biotin ligase is able to biotinylate them, thereby facilitating their enrichment and subsequent identification by MS. In specific embodiments the biotin ligase is fused to a protease, or a subunit of the proteasome such that the protease function of the protease or the function of the assembled proteasome are not affected by the protein fusion. As described herein this biotin ligase-fusion protein preferably enables to biotinylate proteins that interact, are processed and/or come into close proximity, e.g., 100-0,001 nm, or less than 100 nm, of the enzyme or enzyme complex-part of the fusion protein.

“Gene expression” is the process by which information from a gene is used in the synthesis of a functional gene product that enables it to produce end products, such as proteins or non-coding RNA. All steps in the gene expression process may be regulated (modulated), including the gene transcription of DNA into RNA sequence information, RNA splicing, RNA to protein translation, and post-translational modification of a protein. In genetics, the gene expression is the most fundamental level at which the genotype gives rise to the phenotype. In this context the term “encoding” refers to a gene that encodes or codes for a nucleic acid sequence that may be transcribed first to mRNA and finally translated into a protein, in other words a DNA sequence encodes, dependent on splicing of the mRNA, one or more amino acid sequences that can be translated to one or more proteins.

In the context of the present invention the term “proximity” refers to a close distance between two proteins or molecules that interact or come into close proximity to each other, wherein the proximity may refer to a distance between 1 and 100 nm, in some embodiments of less than 15 nm, less than 10 nm, or less than 5 nm, or between 0.001 and 50 nm, or between 0.01 and 10 nm. In preferred embodiments a proximity or close proximity refers to a distance between 10 and 0.001 nm or even more preferably of less than 10 nm.

In biochemistry, the term “biotinylation” refers to the process of covalent binding of biotin to a protein, nucleic acid, or other molecule. Biotinylation is considered to not affect the natural function of the biotinylated molecule due to the small size of biotin (MW=244.31 g/mol). Biotin binds to streptavidin and avidin with extremely high affinity, high speed, and high specificity, and these interactions are generally used to isolate biotinylated molecules of interest, wherein the binding of biotin to streptavidin and avidin is considered to be insensitive to heat, pH, and proteolysis, allowing biotinylated molecules to be isolated in a reliable fashion. Alternatively biotinylated substrates or proteins may be enriched or isolated using an antibody having an affinity to biotin or derivates thereof, namely being directed against biotin or derivates thereof. The small size of biotin enables in certain embodiments the conjugation of multiple biotin molecules to a molecule of interest, which may allow the binding of multiple streptavidin, avidin, or neutravidin molecules and thereby increases the sensitivity of enrichment or detection of the protein of interest. In embodiments the biotin tag can be used in affinity chromatography together with a column or solid phase that comprises preferably avidin, streptavidin or neutravidin or an antibody directed against biotin. Substrates, preferably proteins can be biotinylated chemically or enzymatically. Herein enzymatic biotinylation is preferred and usually results, in the case of protein or peptide targets, in the biotinylation of a lysine within the amino acid sequence of a target protein, for example, by a bacterial biotin ligase. In embodiments Enzymatic biotinylation can be carried out by E. coli biotin holoenzyme synthetase, also known as “biotin ligase” or also termed “biotin conjugating enzyme” (BirA, P06709).

Biotinylation of targets that is independent from specific amino acid-sequences, also called proximity-based biotinylation, can be achieved in embodiments herein by creating a fusion protein of interest that is fused in-frame with a biotin ligase mutant (such as BirA R118G, also termed “BirA*”, or BioID, BioID2, BASU, AirID, microID, microID2, ultraID, TurboID, miniTurbo, APEX or APEX2). Although the BirA* moiety is able to efficiently activate biotin, thereby creating biotinoyl-AMP, it possesses a reduced affinity for it. Due to the mutant ligases' low affinity for biotinoyl-AMP, the biotinoyl-AMP simply diffuses away from the biotin ligase and reacts with amine groups within close proximity (usually less than 100 nm), for example with lysine residues of any nearby localized peptides or proteins. Such mutant biotin ligases are also termed “promiscuous biotin ligases”, as they are non-specific regarding their targets and can mediate the biotinylation of any suitable substrates in a close proximity. In the context of the present invention the term “biotinylation”, besides referring to a modification/attachment of biotin to a substrate, also comprises modifications with derivates of biotin.

Herein biotin ligases or variants thereof are, for example, BirA, BirA*, or BioID, BioID2, BASU, AirID, microID, microID2, ultraID, TurboID, miniTurbo, APEX or APEX2. The E.-coli biotin ligase BirA (P06709) comprising the mutation R118G, named “BirA*” is a promiscuous biotin ligase enzyme facilitating the attachment of biotin not just to a specific target amino acid sequence, but to any suitable amino acid chain in its close proximity of about 10 nm or less. The biotin ligase BioID2 comprises a R40G mutation, which is orthologous to the R118G mutation in BirA*, wherein BioID2 is derived from Aquifex aeolicus. The biotin ligase BASU comprises a deletion of the N-terminal domain (1-65) of the Type-II BPL from Bacillus subtilis (UniprotID: P0C1175) and an R142G mutation, which is orthologous to the R118G in E. coli BirA*, along with two further mutations in the C-terminal domain (E323S, G325R). The promiscuous biotin ligases TurboID and miniTurbo are considered to exhibit a 3-6-fold increase in activity compared with BirA* over a short period of labelling and up to 15-23-fold increase over longer durations (6-18 hours of biotin treatment). These enzymes contain 15 and 13 mutations (plus a N-terminal deletion of the first 63 amino acids for miniTurbo) compared to wild type BirA from E. Coli, wherein TurboID has an additional two mutations (S263P and M241T) (Samavarchi-Tehrani et al., 2020). Accordingly, TurboID contains the following amino acid substitutions relative to the wild-type BirA gene of E. coli: Q65P, 187V, R118S, E140K, Q141R, A146A (deletion), S150G, L151P, V160A, T192A, K1941, M209V, M241T, S263P and 1305V. The promiscuous biotin ligase microID2 comprises a 63AA deletion at the c-terminal domain, L41S, L46F, K36R, and K44R, and K102R. MicroID2 is the smallest biotin ligase with 180 amino acids compared to 257 AA for miniTurbo and 338 AA for TurboID. UltraID is a microID-directed evolution-deduced variant, which similarly possesses a molecular weight below 20 kDa. UltraID is known to label about the same number of substrates in 10 min labelling time as BioID is able to label in 24 hours. Another BirA-derived promiscuous biotin ligase enzyme is AirID (ancestral BirA for proximity-dependent biotin identification), which was designed de novo using an ancestral enzyme reconstruction algorithm and metagenome data. The ascorbic acid peroxidase enzyme(s) APEX(2) is able to convert (exogenously supplied) biotin-phenol to biotin-phenoxyl radicals, which subsequently react with and thereby label (biotinylate) substrates (covalently), such as the electron-rich amino acids of proteins, in a radius of about 10-20 nm.

In some embodiments the peroxidases “APEX” and “APEX2” are comprised by the fusion protein and thereby employed for proximity biotinylation of substrates. The mechanism that is generally understood to underly the biotinylation reaction through peroxidases comprises the oxidation of a phenolic compound that is coupled to biotin (e.g., biotin-phenol), resulting in the activation (in the presence of H2O2) of said compound by producing a short-lived radical that can react, e.g., in case of protein or peptide substrates, with electron-rich amino acids, such as tyrosine. In some embodiments where the fusion protein comprises a peroxidase, such as APEX or APEX2, the cells expressing the fusion protein comprising APEX(2) are first incubated with biotinyl tyramide (biotin-phenol) and subsequently treated with H₂O₂ to initiate proximal labelling.

Herein for the biotinylation or the attachment of biotin to a substrate the chemical molecule biotin or any suitable derivate thereof may be used. In biochemistry in general numerous different biotin derivatives are used as biotinylation reagents. Derivates of biotin referred to herein may comprise molecules selected from the group comprising Tyramide-Biotin (Biotin-Phenol), Desthiobiotin-Phenol, Biotin Arylazide, BxxP, Biotin HPDP, D-(+)-Biotin, D-Desthiobiotin, 3-(N-Maleimidylpropionyl)biocytin, N-Biotinyl-N′-cysteinyl Ethylenediamine Trifluoroacetic Acid Salt, MTSEA Biotin, or N-(+)-Biotinyl-6-aminohexanoic acid, Biotin 5-Bromopentylamide, S-(1-Pentyl-5-biotinylamido)glutathione, Sulpho NHS biotin, N-Biotinyl-6-amino-2-naphthoic Acid, Biotin Azide (PEG4 carboxamide-6-Azidohexanyl Biotin), Biotin-PEG3-Azide, Biotin Picolyl Azide.

To increase the efficiency of substrate/target biotinylation during the present method in preferred embodiments biotin or derivates thereof are supplemented additionally for a certain period of time. In some embodiments “biotin supplementation”, which may in embodiments herein also refer to the supplementation of biotin derivates, may be performed before the biotinylation step, or during the biotinylation step, or before and during the biotinylation step. In embodiments biotin or derivates thereof are supplemented at least 1, 2, 3, 4, 5, 6, 7, 8, 9, 10, 11, 12, 13, 14, 21, 28, 30 days or at least 1, 2, 3, 4, 5, 6, 7, 8, 9, 10, 11, 12, 13, 14, 15, 16, 17, 18, 19, 20, 21, 22, 23, 24, 36, 48, 60, 72, 96, 120 hours or at least 1 minute, 2, 3, 4, 5, 6, 7, 8, 9, 10, 15, 30, 45, or 60 minutes before the biotinylation step and/or during the biotinylation step. In embodiments biotin or derivates thereof are supplemented at least once per week, at least every second day, every day, at least once, twice or three times per day or continuously. Means for administration of biotin or derivates thereof are dependent on the system or organism used in the context of the present invention and known to the skilled person. Non-limiting examples of modes of administration are described herein.

In the context of the present invention a protein- or affinity-tag, may be selected from the group comprising a FLAG-, GFP-, Strep-, GST-, His-, CBP-, CBD-, MBP-, c-Myc-, Halo-, Protein G-, Protein A-, HA-, T7-tag or any other suitable protein- or affinity-tag. In general protein tags consist of peptide sequences genetically grafted onto a recombinant protein, wherein affinity tags are appended to proteins. Both kind of tags facilitate the specific detection, isolation, purification and/or enrichment of the tagged protein or peptide using an affinity technique. GFP-tag is one example of a protein tag that facilitates not just isolation of the tagged protein, but also a direct detection if the tagged protein through fluorescence of the GFP-tag upon excitation. Isolation or enrichment may occur using antibodies directed against the tag or by other agents having an affinity for the tag. Any kind of agent or antibody having an affinity for the tag may be comprised within a resin, a column material, or any other a solid phase, which may be stationary, or mobile such as, e.g., beads or magnetic beads.

Herein an “enzyme complex” may be any molecule comprising at least an enzyme that interacts (is non-covalently linked) with at least one further protein or even a nucleic acid. In preferred embodiments an enzyme complex is a protein complex, or in other words, a group of polypeptide molecules linked by noncovalent protein-protein interactions (PPIs), wherein at least one protein is an enzyme. In specific embodiments the enzyme complex comprises at least one protease. In some specific embodiments the enzyme complex is a proteasomal complex or a fully functional assembled proteasome. In some embodiments the enzyme complex is a fully functional assembled molecule complex, e.g., protein complex, in some embodiments the enzyme complex comprises only a fraction of its subunits required for its natural function.

In some embodiments the protease inhibitor compound may be a small molecule designed to fit into the substrate binding site on the catalytic subunit such as peptide aldehydes (e.g., MG132), peptide boronates (e.g., MG-262, bortezomib, ixazomib, delanzomib), epoxyketones (e.g., epoxomicin, carfilzomib, oprozomib, zetomipzomib, ONX 0914), or nonpeptide p-Lactone (e.g., lactacystin), salinosporamides (e.g., marizomib) and vinyl sulfones.

The term “proteasome” refers to a protein complex that degrades, e.g., unneeded or damaged proteins by proteolysis (a chemical reaction breaking peptide bonds). Enzymes assisting in such reactions are termed proteases. Proteasomes are part of a major mechanism by which cells regulate the concentration of particular proteins and degrade misfolded proteins. Proteins are tagged for degradation with a small protein called ubiquitin. The tagging reaction is catalysed by enzymes called ubiquitin ligases. Once a protein is tagged with a single ubiquitin molecule, this is a signal to other ligases to attach additional ubiquitin molecules. The proteasome is a cylindrical complex containing a “core” of four stacked rings forming a central pore. In some embodiments each ring is composed of seven individual proteins. Preferably, the inner two rings are made of seven p-subunits that contain three to seven protease active sites, which are located on the inner surface of the rings, forcing a target protein to enter the central pore before is degradation. Each of the outer two rings comprises seven a-subunits. These alpha subunits' function is the maintenance of a “gate” structure through which proteins enter the barrel-like structure of the proteasome. Alpha-subunits are regulated by binding to “cap” structures or regulatory particles recognizing polyubiquitin tags on protein substrates and initiating the degradation of said substrates. The subunits of the proteasome are named according to their Svedberg sedimentation coefficient, wherein mammal proteasomes are comprised by the cytosolic 26S proteasome (~2000 kDa). The 26S proteasome comprises one 20S protein subunit and two 19S regulatory cap subunits. An alternative form of regulatory subunit is the 11S particle which associates with the core similar to the 19S particle.

Accordingly, herein a proteasome complex protein may be selected from the group comprising PSMA1-8, PSMB1-11, PSMC1-6, PSMD1-14, PSME1-4, PSMF1, PAMD12, ADRM1, USP14/Ubp6, UCHL5/UCH37, UBE3C/KIAA10/Hul5, SHFM1/DSS1, or any combination thereof.

In specific embodiments the proteasome complex protein is selected from the group consisting of PSMA1-8, PSMB1-11, PSMC1-6, PSMD1-14, PSME1-4, PSMF1, or any combination thereof.

In specific embodiments the proteasome complex protein is selected from the group consisting of PSMA1-8, PSMC1-6 and PSMD1-14 or any combination thereof.

In specific embodiments the proteasome complex protein is selected from the group consisting of PSMA4, PSMC2 and PSMD3 or any combination thereof.

In the context of the present invention a “substrate” and/or “interaction partner” of the enzyme or enzyme complex is any protein or peptide that interacts with the enzyme or enzyme complex, or any subunit of the enzyme complex, and/or is processed or intended to be processed by it. In embodiments wherein the enzyme or enzyme complex is a protease and/or proteasome complex, the substrate and/or interaction partner may be any protein or peptide that interacts with the protease, protease-subunit of the proteasome or one or more subunits of the proteasome, and/or is processed or intended to be processed by it. Substrates that are “intended” to be processed by a protease, or especially the proteasome, might be labelled or chemically modified, e.g., with ubiquitin, whereby the tagging or modification indicates them to be processed by the protease or proteasome whereupon they may by recognized by the protease, the proteasome or specific transport proteins that transport them to the protease or proteasome. In cases where the enzyme, protease or enzyme complex, proteasome, or any protease subunit thereof, are inhibited, no substrate can be processed despite being intended to be processed (which may, for example, be indicated by a tag or a modification), although they will likely still interact or be in the close proximity of the enzyme, enzyme complex, protease or proteasome.

As used herein, “identity”, “sequence identity”, “sequence homology” or “homology” in the context of two nucleic acid sequences refers to a specified percentage of residues (e.g., amino acids or nucleic acids) in the two sequences, which are the same when aligned for maximum correspondence over a specified comparison window, as determined by sequence comparison algorithms or by visual inspection.

As used herein, “percent (%) sequence identity”, “sequences with % identity”, “percent (%) sequence homology” or “sequences with % homology” to a specific “reference sequence” (e.g., to SEQ ID NO. 1) means the percentage of residues (e.g., amino acids or nucleic acids) in a certain sequence that are identical with the residues (e.g., amino acids or nucleic acids) of the ‘reference’ sequence and is determined by comparing the two optimally aligned sequences over a comparison window wherein the portion of the poly-residue (e.g., poly-amino acids or poly-nucleotide) sequences in the comparison window may include additions or deletions (i.e., gaps) as compared to the ‘reference’ sequence (which does not include additions or deletions) for optimal alignment of the two or more sequences. The percentage is calculated by determining the number of positions at which the identical residue, e.g., nucleic acid base or amino acid residue, occurs in the two or more sequences to yield the number of matched positions, dividing the number of matched positions by the total number of positions in the window of comparison, and multiplying the result by 100 to yield the percentage of sequence identity.

Any suitable method of aligning sequences to obtain percent sequence identity may be used and will be known to a skilled person. The assessment of the percentage identity between any two or more sequences may be carried out using a publicly available mathematical algorithm. Computer software implementations of such mathematical algorithms comprise without being limited thereto: ClustalW algorithm (VNTI software, InforMax Inc.), BLAST®, ALIGN, GAP (Genetics Computer Group, available via Accelrys), Multiple sequence alignments MUSCLE (EMBL's European Bioinformatics Institute, UK), BESTFIT, FASTA and TFASTA in the Wisconsin Genetics Software Package (Genetics Computer Group, USA), and Algorithms for database searching are typically based on the BLAST software (Altschul et al., 1990). Alignments using these programs can be performed using the default parameters. Software for performing BLAST® analyses is publicly available through the US National Center for Biotechnology Information (NCBI). A sequence database can be searched using the nucleic acid sequence of interest. In some embodiments, the percent homology or identity can be determined along the full-length of the nucleic acid.

In embodiments a polypeptide (e.g., fusion protein) and/or a nucleic acid encoding the same, as described herein, comprises or consists (or encodes) of an amino acid sequence according to SEQ ID NO 1-4, or variants and/or fragments of said sequences, wherein the sequence variant and/or fragment may comprise a sequence identity to SEQ ID NO 1-4 of 50, 55, 60, 65, 70, 75, 80, 81, 82, 83, 84, 85, 86, 87, 88, 89, 90, 91, 92, 93, 94, 95, 96, 97, 98, 99, 99.5 or 99.9%.

Sequence identity may be determined using methods known to one skilled in the art, such as ClustalW or BLAST or other sequence alignment tools.

Herein, a genetically modified animal or cell is a genetically engineered organism (GMO) which genetic material has been altered by adding, changing or removing DNA sequences using genetic engineering techniques.

Tetracycline-controlled transcriptional activation is a method of inducible gene expression where transcription is reversibly turned on or off in the presence of the antibiotic tetracycline or one of its derivatives (e.g., doxycycline). Tetracycline-controlled gene expression is based on the tetracycline antibiotic-resistance mechanism of gram-negative bacteria. In general, the difference between “Tet-On” and “Tet-Off” is not related to the activation or silencing of a gene by the transactivator, but rather, both proteins activate expression. The difference relates to the respective response to tetracycline or doxycycline (doxycycline, is a more stable tetracycline analogue); Tet-Off activates expression in the absence of doxycycline, whereas Tet-On activates in the presence of doxycycline.

In general, “mass spectrometry” (MS) refers to a technology for separation of electrically charged molecules (ion) in the gas phase. The ions are preferably produced in an ion source, e.g., electro spray ionization (ESI), which enables the transfer of both solid-phase or liquid-phase (e.g., from liquid chromatography; LC) analytes into the gas phase. These gas-phase ions are then analyzed by a mass analyser that sorts the ions, in space or time, according to their mass to-charge ratio (m/z). Finally, the separated ions are detected as electrical signals by an ion detector in the space or time domain and are translated into mass spectra (showing the number of ions at different m/z values). Therefore, mass spectrometry facilitates the identification of molecules based on their characteristic mass-to-charge ratio and fragmentation patterns. In “tandem mass spectrometry”, also termed “MS/MS”, two or more mass analysers are coupled together, applying an additional reaction step to increase the resolution of sample analyse. In a first step during MS/MS the molecules of a sample are ionized, and the first spectrometer (called MS1) separates these ions by their mass-to-charge ratio (m/z). In a second step, ions of a certain m/z-ratio are selected and then fragmented, e.g., by collision-induced dissociation, higher-energy collision dissociation (HCD), electron-transfer dissociation (ETD), electron capture dissociation (ECD), ion-molecule reaction, or (ultraviolet) photodissociation. Subsequently, the fragments are introduced into the second mass spectrometer (MS2), to separate the fragments by their m/z-ratio and finally detect them. The separation and fragmentation increase the resolution of the detection, as it facilitates the separation and identification of ions with very similar m/z-ratios in single mass spectrometry (MS).

Quantitative mass spectrometry can be used for quantitative proteomics, namely for determining the amount of proteins in a sample. For MS there exists methods for relative and absolute quantification. An example of relative quantification comprises labelling the samples with stable isotope labels that allow the distinction between identical proteins in different samples. Examples of such relative quantification methods are isotope-coded affinity tags (ICAT), stable isotope labelling with amino acids in cell culture (SILAC), isobaric labelling (tandem mass tags (TMT) and isobaric tags for relative and absolute quantification (iTRAQ)), N-terminal labelling, terminal amine isotopic labelling of substrates (TAILS) and label-free quantification metal-coded tags (MeCAT). In label-free approaches different samples are analyzed separately followed by comparison of their mass spectra, whereby the abundance of peptides in each sample is determined relative to each other. Label-free quantification is usually either based on precursor signal intensity or on spectral counting. During area under the curve (AUC) methods for each peptide spectrum of a LC-MS run, the area under the spectral peak is calculated, which is linearly proportional to the concentration of protein in the analysed sample. During spectral counting the spectra of an identified protein are counted and standardized applying an applicable normalization.

The analysis of mass spectrometry raw data can involve data independent acquisition (DIA) or data dependent acquisition. In comparison to data-dependent acquisition (DDA), in which precursors are selected by intensity for fragmentation, in DIA, all precursors within specified mass ranges (so called precursor isolation windows) are fragmented. In general, during MS1 acquisition the complete mass range, which is usually located between 350-1,650 m/z, is scanned. In a DIA method, precursor isolation windows are set across the complete mass range for MS2 acquisition. The number and width (fixed or variable) of the windows depend on the LC gradient and instrument. All peptides within an isolation window are simultaneously fragmented and subsequently detected by MS/MS. The identity of the peptides can be obtained by matching the ion peaks in a mass spectrum to a spectral library, comprising information on a peptide fragment ions' pattern and its elution time from LC. The method can be improved further by coupling ion mobility spectrometry (high field asymmetric waveform or trapped) prior to the MS analysis.

Liquid chromatography (LC) is a method of physical separation in which the components of a liquid mixture are distributed between two immiscible phases, i.e., stationary and mobile. The practice of LC can be divided into five categories, i.e., adsorption chromatography, partition chromatography, ion-exchange chromatography, size-exclusion chromatography, and affinity chromatography. Among these, the most widely used variant is the reverse-phase (RP) mode of the partition chromatography technique, which makes use of a nonpolar (hydrophobic) stationary phase and a polar mobile phase. High-performance liquid chromatography (HPLC), is a method used to separate, identify, and quantify multiple components of a sample. It uses pumps to pass a pressurized liquid solvent containing the sample through a column filled with a solid adsorbent material, wherein each component in the sample interacts slightly differently with the adsorbent material, causing different flow rates for the different components and leading to the separation of the components as they flow out of the column.

In embodiments an “on-bead” digestion describes the digestion of enriched or isolated target proteins that are still bound to the enrichment-phase, which is preferably a solid phase comprising molecules with a high affinity for biotin, e.g., streptavidin, avidin, NeutrAvidin or an antibody directed against biotin. The on-bead digestion is preferably conducted before the mass spectrometry measurement of the enriched or isolated target proteins. The on-bead digestion is preferably a proteolysis by the enzyme(s) trypsin and/or LysC. Preferably the digested target proteins are subsequently eluted from the solid phase and analyzed by mass spectrometry, preferably combined with liquid chromatography previous to mass spectrometry. In embodiments the peptides that are created by the on-bead digestion and are “free” floating in solution (not bound to the solid phase) are analyzed separately from the peptides, that are still bound to the solid phase after on-bead digestion (as they comprise at least one biotin bound to the solid phase). These biotinylated peptides are acquired by elution from the solid phase, e.g., in embodiment by acetonitrile (ACN) and/or trifluoroacetic acid (TFA).

In embodiments of the present invention a “candidate substance” may be any substance of interest. Preferably the present method is employed to analyse the effect of a substance of interest on the protein-protein interactions of and/or substrate-recruitment to the enzyme or enzyme complex. Accordingly, in some embodiments the substance of interest/candidate substance is a chemical compound, a pharmaceutical compound or a drug. In some specific embodiments the substance of interest/candidate substance is suspected to influence the protein-protein interactions of and/or substrate-recruitment to the enzyme or enzyme complex, wherein in specific embodiments the enzyme is a protease and/or the enzyme complex is a part of, or an entire functional proteasome. Herein the terms “substance” or “compound may be used interchangeably.

In the context of protein interactions or substrates of the enzyme or enzyme complex, the term “substrate” refers to a molecule which is suspected or supposed to be enzymatically processed by the enzyme or enzyme complex. In specific embodiments the enzyme or enzyme complex is or comprises a protease and the substrate is enzymatically digested by the enzyme or the enzyme-subunit of the enzyme complex. In some specific embodiments the enzyme or enzyme complex is a part of, or an entire functional proteasome and the substrate is suspected or supposed to be digested or processed by the proteasome or subunits thereof.

Herein, preferred means for cell-transfection, transduction, or infection or gene knock-in are those commonly known to a skilled person. Suitable methods are available and do not require undue effort to establish

The term “at least one” may herein refer to at least one, more than one, at least two at least three, at least four, at least five, at least six, at least seven, at least eight, at least nine, at least ten, at least fifteen, at least twenty, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 500, 1000, 10.000.

### Sequences

Preferred sequences of the invention are shown in the following Table 1:

| SEQ ID | | |
|---|---|---|
| NO. | Sequence | Fusion protein |
| 1 | >PSMA4_BirA | Amino acid |
| | MSRRYDSRTTIFSPEGRLYQVEYAMEAIGHAGTCLGILANDGVLLAAER | sequence of |
| | RNIHKLLDEVFFSEKIYKLNEDMACSVAGITSDANVLTNELRLIAQRYL | PSMA4 |
| | LQYQEPIPCEQLVTALCDIKQAYTQFGGKRPFGVSLLYIGWDKHYGFQL | fused to BirA; |
| | YQSDPSGNYGGWKATCIGNNSAAAVSMLKQDYKEGEMTLKSALALAIKV | Sequences |
| | LNKTMDVSKLSAEKVEIATLTRENGKTVIRVLKQKEVEQLIKKHEEEEA | belonging to |
| | KAEREKKEKEQKEKDKYPTFLYKVVDSRMKDNTVPLKLIALLANGEFHS | PSMA4 are |
| | GEQLGETLGMSRAAINKHIQTLRDWGVDVFTVPGKGYSLPEPIQLLNAK | underlined, |
| | QILGQLDGGSVAVLPVIDSTNQYLLDRIGELKSGDACIAEYQQAGRGGR | sequences belonging to |
| | GRKWFSPFGANLYLSMFWRLEQGPAAAIGLSLVIGIVMAEVLRKLGADK | BirA are italic. |
| | VRVKWPNDLYLQDRKLAGILVELTGKTGDAAQIVIGAGINMAMRRVEES | |
| | VVNQGWITLQEAGINLDRNTLAAMLIRELRAALELFEQEGLAPYLSRWE | |
| | KLDNFINRPVKLIIGDKEIFGISRGIDKQGALLLEQDGIIKPWMGGEIS | |
| | LRSAEKGGSGPGGGAPDYKDDDDK | |
| 2 | >PSMC2_BirA | Amino acid |
| | MPDYLGADQRKTKEDEKDDKPIRALDEGDIALLKTYGQSTYSRQIKQVE | sequence of |
| | DDIQQLLKKINELTGIKESDTGLAPPALWDLAADKQTLQSEQPLQVARC | PSMC2 fused |
| | TKIINADSEDPKYIINVKQFAKFVVDLSDQVAPTDIEEGMRVGVDRNKY | to BirA; |
| | QIHIPLPPKIDPTVTMMQVEEKPDVTYSDVGGCKEQIEKLREVVETPLL | Sequences |
| | HPERFVNLGIEPPKGVLLFGPPGTGKTLCARAVANRTDACFIRVIGSEL | belonging to |
| | VQKYVGEGARMVRELFEMARTKKACLIFFDEIDAIGGARFDDGAGGDNE | PSMC2 are |
| | VQRTMLELINQLDGFDPRGNIKVLMATNRPDTLDPALMRPGRLDRKIEF | underlined, |
| | SLPDLEGRTHIFKIHARSMSVERDIRFELLARLCPNSTGAEIRSVCTEA | sequences |
| | GMFAIRARRKIATEKDFLEAVNKVIKSYAKFSATPRYMTYNCPTFLYKV | belonging to |
| | VDSRMKDNTVPLKLIALLANGEFHSGEQLGETLGMSRAAINKHIQTLRD | BirA are italic. |
| | WGVDVFTVPGKGYSLPEPIQLLNAKQILGQLDGGSVAVLPVIDSTNQYL | |
| | LDRIGELKSGDACIAEYQQAGRGGRGRKWFSPFGANLYLSMFWRLEQGP | |
| | AAAIGLSLVIGIVMAEVLRKLGADKVRVKWPNDLYLQDRKLAGILVELT | |
| | GKTGDAAQIVIGAGINMAMRRVEESVVNQGWITLQEAGINLDRNTLAAM | |
| | LIRELRAALELFEQEGLAPYLSRWEKLDNFINRPVKLIIGDKEIFGISR | |
| | GIDKQGALLLEQDGIIKPWMGGEISLRSAEKGGSGPGGGAPDYKDDDDK | |
| 3 | >BirA_PSMD3 | Amino acid |
| | MKDNTVPLKLIALLANGEFHSGEQLGETLGMSRAAINKHIQTLRDWGVD | sequence of |
| | VFTVPGKGYSLPEPIQLLNAKQILGQLDGGSVAVLPVIDSTNQYLLDRI | PSMD3 fused |
| | GELKSGDACIAEYQQAGRGGRGRKWFSPFGANLYLSMFWRLEQGPAAAI | to BirA; |
| | GLSLVIGIVMAEVLRKLGADKVRVKWPNDLYLQDRKLAGILVELTGKTG | Sequences |
| | DAAQIVIGAGINMAMRRVEESVVNQGWITLQEAGINLDRNTLAAMLIRE | belonging to |
| | LRAALELFEQEGLAPYLSRWEKLDNFINRPVKLIIGDKEIFGISRGIDK | PSMD3 are |
| | QGALLLEQDGIIKPWMGGEISLRSAEKGGSGPGGGAPDYKDDDDKLGAP | underlined, |
| | TSLYKKVGMKQEGSARRRGADKAKPPPGGGEQEPPPPPAPQDVEMKEEA | sequences |
| | ATGGGSTGEADGKTAAAAAEHSQRELDTVTLEDIKEHVKQLEKAVSGKE | belonging to |
| | PRFVLRALRMLPSTSRRLNHYVLYKAVQGFFTSNNATRDFLLPFLEEPM | BirA are italic. |
| | DTEADLQFRPRTGKAASTPLLPEVEAYLQLLVVIFMMNSKRYKEAQKIS | |
| | DDLMQKISTQNRRALDLVAAKCYYYHARVYEFLDKLDVVRSFLHARLRT | |
| | ATLRHDADGQATLLNLLLRNYLHYSLYDQAEKLVSKSVFPEQANNNEWA | |
| | RYLYYTGRIKAIQLEYSEARRTMTNALRKAPQHTAVGFKQTVHKLLIVV | |
| | ELLLGEIPDRLQFRQPSLKRSLMPYFLLTQAVRTGNLAKFNQVLDQFGE | |
| | KFQADGTYTLIIRLRHNVIKTGVRMISLSYSRISLADIAQKLQLDSPED | |
| | AEFIVAKAIRDGVIEASINHEKGYVQSKEMIDIYSTREPQLAFHQRISF | |
| | CLDIHNMSVKAMRFPPKSYNKDLESAEERREREQQDLEFAKEMAEDDDD | |
| | SFP | |
| 4 | >PSMA4_miniTurboID | Amino acid |
| | MSRRYDSRTTIFSPEGRLYQVEYAMEAIGHAGTCLGILANDGVLLAAER | sequence of |
| | RNIHKLLDEVFFSEKIYKLNEDMACSVAGITSDANVLTNELRLIAQRYL | PSMA4 fused |
| | LQYQEPIPCEQLVTALCDIKQAYTQFGGKRPFGVSLLYIGWDKHYGFQL | to miniTurbo; |
| | YQSDPSGNYGGWKATCIGNNSAAAVSMLKQDYKEGEMTLKSALALAIKV | Sequences |
| | LNKTMDVSKLSAEKVEIATLTRENGKTVIRVLKQKEVEQLIKKHEEEEA | belonging to |
| | KAEREKKEKEQKEKDKYPTFLYKVVDSRMASIPLLNAKQILGQLDGGSV | PSMA4 are |
| | AVLPVVDSTNQYLLDRIGELKSGDACIAEYQQAGRGSRGRKWFSPFGAN | underlined, |
| | LYLSMFWRLKRGPAAIGLGPVIGIVMAEALRKLGADKVRVKWPNDLYLQ | sequences |
| | DRKLAGILVELAGITGDAAQIVIGAGINVAMRRVEESVVNQGWITLQEA | belonging to |
| | GINLDRNTLAAMLIRELRAALELFEQEGLAPYLSRWEKLDNFINRPVKL | miniTurbo are |
| | IIGDKEIFGISRGIDKQGALLLEQDGVIKPWMGGEISLRSAEKLQDDYK | italic. |
| | DDDDK | |

### FIGURES

The invention is further described by the following figures. These are not intended to limit the scope of the invention but represent preferred embodiments of aspects of the invention provided for greater illustration of the invention described herein.

FIG. 1: Establishment of a cell culture model system for proximity labeling of proteasomes

This Figure depicts one embodiments of the present invention.

A. Schematic representation of proteasome and location of components fused to biotin ligase (bait proteins. Highlighted in black for 20S and 19S proteasome).

B. Left panel, immunoblot of BirA* fusion proteins performed on lysates from HEK293T cells stably transfected with PSMA4-BirA*-FLAG, PSMC2-BirA*-FLAG or BirA*-PSMD3-BirA* following 24 hours incubation with (+tet) or without (−tet) tetracycline. Right panel, streptavidin-HRP blot following induction of BirA* fusion proteins with tetracycline and supplementation of biotin for 24 hours. Amido Black or Ponceau S staining was used as loading control. HRP: horseradish peroxidase.

C. Immunofluorescence analysis of PSMA4-BirA*-FLAG cell line 4 days after seeding without addition of any substance (−tet −bio), with addition of only tetracycline for 4 days (+tet −bio) or with addition of both tetracycline for 4 days and biotin for 1 day (+tet +bio). Scale bar indicates 20 μm.

D. Comparison of expression levels of PSMA4-BirA* (lanes marked by star) and its endogenous counterpart (lanes marked by arrowhead), following 24 hours incubation with (+tet) or without (−tet) tetracycline. Ponceau S staining was used as loading control.

E. Proteasome activity assay performed on lysates from cell lines expressing different BirA* fusion proteins, following 24 hours incubation with (+tet) or without (−tet) tetracycline. Equal amounts of protein extracts were incubated with proteasome substrate LLVY-7-Amino-4-methylcoumarin (AMC) and substrate cleavage assessed by fluorimetry. n=3 biological replicates, paired t-test.

F. Size exclusion chromatography (SEC) analysis of lysates from HEK293T cells stably expressing PSMA4-BirA* following 24 hours incubation with tetracycline. SEC fractions were analyzed by DIA mass spectrometry and elution profiles were built for each protein using protein quantity values normalized to the sum of quantities across all fractions. Depicted are elution profiles of PSMA4 (proteasome subunit, black solid line) and BirA* (biotinylating enzyme, dashed line). The peaks corresponding to different proteasome assemblies were assigned based on the elution profiles of other proteasome components.

FIG. 2: Use of an embodiment of the present method

This Figure depicts one embodiments of the present invention.

A. Principal Component Analysis (PCA) of BioID data obtained from cell lines expressing BirA*-fused proteasome subunits and control (BirA*). The smaller dots represent individual samples and the larger dots the centroids of each group. Ellipses represent 95% confidence intervals. The percentage of variance explained by the first two principal components (PC) axes is reported in the axis titles. n=4, biological replicates.

B. Volcano plots of proteins enriched by streptavidin pull-down and analyzed by DIA mass spectrometry. Cut offs for enriched proteins: log 2 fold change>1 and Q value<0.05. n=4, biological replicates.

C. Level of enrichment of proteasome subunits measured by the present invention in the context of the proteasome structure. Enriched proteins are depicted in different shades of red according to the log 2 fold enrichment vs. BirA* control.

D. Biotinylated protein sites identified by the method according to one embodiment of the present invention. Highlighted with arrow head are all residues located within a 10 nm radius of the BirA*-modified PSMA4 subunit. Only the structure of the modified subunit is depicted with a surface model and all the other subunits are depicted as helix-loop structures. Hand pointer indicates the C-terminus of PSMA4 and the identified biotinylated residues are depicted in orange. Biotinylated residues were obtained from the ACN fraction of PSMA4-BirA*. The proteasome structures depicted in C. and D. were obtained from the PDB:5T0C model of the human 26S proteasome (Chen et al. 2016) and rendered using Chimera (Pettersen et al. 2004).

E. Comparison of co-elution profiles obtained by SEC-MS and proteins enriched in the samples according to one embodiment. Pearson correlation values were calculated between PSMA4 and all the other proteins quantified in SEC-MS (n=4680). Correlation values were compared between proteins significantly enriched in PSMA4-BirA* vs. BirA* (log 2 fold change>1 and Q value<0.05) and all the other proteins quantified in the present experiment. P value was calculated using a Wilcoxon Rank Sum test with continuity correction.

F. Venn diagram showing the overlap between proteins significantly enriched in by the method according to the invention and proteasome-interacting proteins identified by Protein Correlation Profiling (PCP) in an independent study (Fabre et al. 2015). The present data were filtered for log 2 fold change>1 and Q value<0.05 vs. BirA* control.

FIG. 3: The present method enables identification of potential novel interactors of the proteasome

This Figure depicts one embodiments of the present invention.

A., B. Network analysis of top 125 interactors of PSMA4-BirA* (A.) and PSMC2-BirA* (B.) obtained according to one embodiment of the present invention. Identified proteins were filtered for significance (Q value<0.05) and then sorted according to the log 2 fold changes of enrichment vs. BirA* control cell line. The size of the nodes depicts the median protein quantity across samples (an estimate of protein absolute abundance), while the color indicates the log 2 fold change vs. BirA* control. The right panel highlights groups of proteins belonging to the same protein complex or biological process.

C. Barplot comparing the levels of SYNJ1 protein following streptavidin enrichment from different cell lines. Protein quantities were derived from DIA mass spectrometry data. n=4 biological replicates.

D. SEC-MS analysis comparing elution profiles of PSMA4 (solid black line), BirA* (dashed gray line) and SYNJ1 (black dashed line). SEC fractions were obtained from lysates of HEK293T cells expressing PSMA4-BirA* and analyzed by DIA mass spectrometry. Elution profiles were built for each protein using protein quantity values normalized to the sum of quantities across all fractions.

E. Heatmap showing the relative abundance of phospho-inositol phosphatases quantified in the present samples. Protein quantities obtained from DIA mass spectrometry were z-transformed for display purposes.

FIG. 4: The present method identifies SYNJ1 protein as novel interactor

This Figure depicts one embodiments of the present invention.

A. Volcano plot of proteins enriched by streptavidin pull-down and analyzed by DIA mass spectrometry from SYNJ1-BirA* and BirA* control cell lines. Cut offs for enriched proteins: log 2 fold change>1 and Q value<0.05. n=4, biological replicates.

B. Heatmap showing the relative abundance of SYNJ1, known SYNJ1-interacting proteins, and proteasome subunits found to be enriched (log 2 fold change>1 and Q value<0.05) in the samples from either SYNJ1-BirA* or BirA*-SYNJ1- vs. BirA* control. Protein quantities obtained from DIA mass spectrometry were z-transformed for display purposes.

C. Overlap of significantly enriched proteins (log 2 fold change>1 and Q value<0.05) in proteasome and SYNJ1 BioID samples vs. BirA* control. Proteins found to be enriched in all the proteasome and SYNJ1 samples are highlighted in a network representation. Nodes colored in grey indicate a group of proteins related to vesicular transport. Nodes highlighted in black are proteasome subunits.

D. Immunofluorescence analysis of PLA experiment performed on PSMA4-BirA* cell after 24 hours incubation with 1 μg/ml tetracycline, as well as on U2OS cells. Scale bar indicates 20 μm.

FIG. 5: Establishment of a mouse model for in vivo use

This Figure depicts one embodiments of the present invention.

A. Design of a mouse model according to one embodiment according to the invention. The lox-STOP-lox cassette was excised from the Rosa26 locus by crossing with a mouse line expressing the Cre recombinase under the control of an ubiquitous CMV promoter (Nagy 2000). CAG: CAG promoter (Miyazaki et al. 1989), TRE: tetracycline-regulated element; rtTA3: reverse tetracycline-dependent transactivator A3 (Dow et al. 2014). (Figure created with biorender).

B. Scheme of an in vivo application of the present invention. D: day, S: sacrifice.

C. Bodyweight curves of the experimental animals. The body weight for each mouse was normalized to its value at day 1 of the experiment (set to 1). M: male, F: female.

D. Barplot comparing the protein levels of miniTurbo (dark gray bars) and PSMA4 (light gray bars) in organs of mice fed with regular chow (−Dox, open bars) or doxycycline-containing food (+Dox, filled bars). Protein quantities were derived from DIA mass spectrometry data obtained from whole organ lysates. n=4, animals per experimental group.

E. Volcano plots of proteins enriched by streptavidin pull-down and analyzed by DIA mass spectrometry from different mouse organs. Cut offs for enriched proteins: log 2 fold change>1 and Q value<0.05. n=4, animals per experimental group.

F. Venn diagram showing the overlap between proteins significantly enriched in the samples from mouse organs, HEK293T cells (either PSMA4-BirA* or PSMC2-BirA*), and proteasome-interacting proteins retrieved from the literature. The data obtained according to the present invention were filtered for log 2 fold change>1 and Q value<0.05 vs. BirA* or miniTurbo control.

FIG. 6: The present method facilitates the identification endogenous and PROTAC-induced proteasome substrates

This Figure depicts one embodiments of the present invention and the results of Example 1.

A. Scheme of the workflow according to one embodiment of the invention in HEK293T cells including proteasome inhibition by MG132. PSMA4-miniTurbo expression and incorporation into proteasomes is achieved by 4 day-induction with tetracycline. Proteasome inhibition is achieved by addition of 20 μM MG132 4 hours before the cell harvesting. Biotin substrate for miniTurbo is supplied 2 hours before the cell harvesting. D: day; h: hour; Tet: tetracycline; Bio: biotin.

B. PCA of BioID data obtained from cell lines expressing PSMA4-miniTurbo and control (miniTurbo), and PSMA4-miniTurbo following exposure to proteasome inhibitor MG132. The smaller dots represent individual samples and the larger dots the centroids of each group.

Ellipses represent 95% confidence intervals. The percentage of variance explained by the first two principal components (PC) axes is reported in the axis titles. n=4, biological replicates.

C., D. Barplots comparing the levels of proteasome activators and ubiquitin (C.) and known proteasome substrates (D.) following streptavidin enrichment from different cell lines and following proteasome inhibition by MG132. Protein quantities were derived from DIA mass spectrometry data. n=4 biological replicates. mT: miniTurbo control cell line; A4-mT: PSMA4-miniTurbo cell line; I: proteasome inhibition by MG132.

E. Scheme of a workflow according to one embodiment of the invention in HEK293T cells including proteasome inhibition by MG132 and treatment with PROTAC KB02-JQ1. The experimental design is analogous to the one depicted in (A.) with the additional PROTAC treatment achieved by addition of 10 μM KB02-JQ1 12 hours before cell harvesting. D: day; h: hour; Tet: tetracycline; Bio: biotin.

F. PCA of BioID data obtained from cells expressing PSMA4-miniTurbo exposed to the proteasome inhibitor MG132 and/or the PROTAC KB02-JQ1. The smaller dots represent individual samples and the larger dots the centroids of each group. Ellipses represent 95% confidence intervals. The percentage of variance explained by the first two principal components (PC) axes is reported in the axis titles. n=4, biological replicates.

G. Barplots comparing the levels of BRD-containing proteins following streptavidin enrichment from PSMA4-miniTurbo expressing cells exposed to the proteasome inhibitor MG132 and/or the PROTAC KB02-JQ1. n=4 biological replicates. mT: miniTurbo control cell line; A4-mT: PSMA4-miniTurbo cell line; I: proteasome inhibition by MG132; P: PROTAC (KB02-JQ1).

FIG. 7: Immunofluorescence validation of PSMC2-BirA* and BirA*-PSMD3 expressing cell lines

This Figure depicts one embodiments of the present invention.

Immunofluorescence analysis of PSMC2-BirA*-FLAG and BirA*-PSMD3-FLAG cell line 4 days after seeding without addition of any substance (−tet −bio), with addition of only tetracycline for 4 days (+tet −bio) or with addition of both tetracycline for 4 days and biotin for 1 day (+tet +bio).

Scale bar indicates 20 μm.

FIG. 8: Optimization of the “BioID” workflow

This Figure depicts one embodiments of the present invention.

A. Scheme of optimized BioID protocol. The original protocol by Mackmull et al. (Mackmull et al. 2017) was optimized herein by (i) acetylation of lysines on streptavidin prior to pull-down; (ii) replacement of trypsin by LysC for on-bead digestion. Following on-bead digestion, two sequential elutions with ammonium bicarbonate (AmBic) and acetonitrile (ACN)/trifluoroacetic acid (TFA) are performed and eluates are further digested off-beads with trypsin. (iii) Peptides from AmBic and ACN/TFA eluates are then analyzed by Data Independent Acquisition (DIA) mass spectrometry.

B. Representative base peak chromatograms of AmBic elutions obtained from the original (upper panel,) and modified (lower panel) BioID protocol. The replacement of trypsin with LysC together with the acetylation of streptavidin drastically reduces the contamination by streptavidin-derived peptides.

C. Quantification of streptavidin in AmBic elutions obtained from the original (T: trypsin) and modified (LT: LysC followed by trypsin) BioID protocol. Streptavidin quantification was based on iBAQ values (Cox & Mann 2008) obtained label free mass spectrometry analysis. Two representative replicates (R1, R2) are shown for each condition.

D. Barplots of the number of identified protein groups and biotinylated peptides obtained using the original vs. optimized BioID protocols. Data were obtained from cell lines expressing PSMA4-BirA* (light gray full dots) or BirA* (black/white dots). The number of identified protein groups was obtained from AmBic samples, while the number of biotinylated peptides was derived from ACN/TFA samples. n=4, biological replicates.

FIG. 9: Validation of cell lines for SYNJ1 BioID

This Figure depicts one embodiments of the present invention.

A. Immunoblot of BirA* fusion proteins performed on lysates collected from HEK293T cells stably transfected with SYNJ1-BirA*-FLAG or BirA*-FLAG-SYNJ1 following 24 hours incubation with (+tet) or without (−tet) tetracycline. Arrows indicate the protein band corresponding to endogenous, untagged SYNJ1 protein, and stars indicate BirA* fusion proteins. Ponceau S staining and immunoblot against beta actin were used as loading controls.

B. Streptavidin-HRP blot following induction of BirA* fusion proteins with tetracycline and supplementation of biotin for 24 hours. Ponceau S staining and immunoblot against beta actin were used as loading controls. HRP: horseradish peroxidase.

C., D. Immunofluorescence analysis of SYNJ1-BirA*-FLAG (C.) and BirA*-FLAG-SYNJ1 (D.) cell lines 4 days after seeding without addition of any substance (−tet −bio), with addition of only tetracycline for 4 days (+tet −bio) or with addition of both tetracycline for 4 days and biotin for 1 day (+tet +bio). Scale bar indicates 20 μm.

FIG. 10: Application of the mouse model according to the invention

This Figure depicts one embodiments of the present invention.

A. Representative agarose gel illustrating successful excision of loxP-STOP-loxP (LSL) cassette by recombination at the CAGs-LSL-rtTA3 locus. Bands highlighted in the dotted square are PCR products amplified from the locus with an excised LSL cassette from two representative mice (no. 51 and no. 52). “0%” and “80%” samples represent control samples from mice showing no excision or approximately 80% excision of the LSL cassette, while “-” sample is a negative control with DNase free water added instead of the DNA template.

B. Immunofluorescence analysis of liver tissue from mice according to one embodiment of the invention fed with regular chow (left) or doxycycline containing food (right) for 14 days and submitted to 7 daily biotin injections. Scale bar indicates 20 μm. DOX: mice fed with doxycycline containing food; noDOX—mice fed with regular chow.

FIG. 11: Validation of PSMA4-miniTurbo cell line and identification of proteasome substrates

This Figure depicts one embodiments of the present invention.

A. Immunoblot of miniTurbo fusion proteins performed on lysates collected from HEK293T cells stably transfected with PSMA4-miniTurbo-FLAG or miniTurbo-FLAG following 4 days of incubation with (+tet) or without (−tet) tetracycline. Immunoblot against GAPDH was used as loading control.

B. Streptavidin-HRP blot following induction of miniTurbo fusion proteins with tetracycline and supplementation of biotin for 2 hours. Immunoblot against GAPDH was used as loading control.

C. Volcano plot of proteins enriched by streptavidin pull-down and analyzed by DIA mass spectrometry from PSMA4-miniTurbo and miniTurbo control cell lines. Cut offs for enriched proteins: log 2 fold change>1 and Q value<0.05. n=4, biological replicates.

D. Comparison of log 2 fold changes for streptavidin-enriched proteins from PSMA4-BirA* and PSMA4-miniTurbo compared to their respective controls. Proteins significant (Q value<0.05) and displaying a log 2 fold change>0 in both comparisons were considered for the analysis.

E. Venn diagram showing the overlap between proteins significantly enriched in the samples (according to embodiment of the invention) (PSMA4-miniTurbo cells) with or without treatment with proteasome inhibitor MG132 (1). The data were filtered for log 2 fold change>1 and Q value<0.05 vs. miniTurbo control.

F. Comparison of log 2 fold protein changes from the whole proteome analysis of HEK293T cells expressing PSMA4-miniTurbo upon MG132 treatment. Displayed log 2 fold changes are averages of n=4 biological replicates. P value was calculated using a Wilcoxon Rank Sum test with continuity correction. Potential proteasome substrates as defined in panel e are highlighted in black.

G. Volcano plot of proteins enriched by streptavidin pull-down and analyzed by DIA mass spectrometry from PSMA4-miniTurbo cells treated with KB02-JQ1 PROTAC molecule (P) and PSMA4-miniTurbo cells treated with both PROTAC molecule (P) and MG132 proteasome inhibitor (1). Cut offs for enriched proteins: log 2 fold change>1 and Q value<0.05. n=4, biological replicates. Enrichment of BRD containing proteins is highlighted in violet boxes.

FIG. 12: The Figure depicts a general concept known in the prior art (e.g., Roux et al. 2012; JCB) of proximity labelling by employing a promiscuous biotin ligase, such as BirA*, fused to a “bait” protein. Wherein the promiscuous biotin ligase biotinylates any protein substrate within the close proximity of a radius of about 10 nm. Thereby, any interactors of the bait protein are biotinylated, if they come into close proximity to the fusion protein. The labelling occurs in situ while it preserves the cellular context and captures also transient and low abundant interactions.

### EXAMPLES

The invention is further described by the following examples. These are not intended to limit the scope of the invention but represent preferred embodiments of aspects of the invention provided for greater illustration of the invention described herein.

### SUMMARY

### Proximity Labeling Coupled to Mass Spectrometry Enables In Situ Mapping of Protein-Protein Interactions.

One example for the various applications of the present invention is presented in the following, wherein it is demonstrated that biotin ligases can be incorporated in fully assembled proteasomes without negative impact on proteasome activity.

In the present example the analysis of proteins labeled by tagged proteasomes retrieved more than half of the known proteasome-interacting proteins in a single mass spectrometry analysis, including assembly factors, activators and ubiquitin-cycle related proteins. By optimizing the protocol for processing of proximity labeled samples and implementing Data Independent Acquisition (DIA) in their mass spectrometry workflow for label-free analysis, the inventors surprisingly achieved an increased number of identified proteins and a significant minimization of contamination from the streptavidin-enrichment step.

The present example demonstrates the utility of the present method for identifying novel proteasome-interacting proteins, charting interactomes across mouse organs, thereby proving that the present approach for proximity-labeling can be successfully used, for example, to identify both endogenous and small molecule-induced proteasome substrates. The present example outlines one embodiment of the method according to the invention wherein the inventors have developed a strategy based on tagging of proteasomes with promiscuous biotin ligases and a newly generated mouse model to monitor the interactome of proteasomes in vivo.

The present example shows how the present invention can be used as an improved method for quantitative mapping of protein-protein interactions (interactomes) of an enzyme complex (here the proteasome) and enzyme substrates (here substrates of the protease subunit of the proteasome) in the context of in vitro and in vivo studies. In the embodiment shown here the present method allows detection of both endogenous proteasome substrates and substrates delivered to the proteasome machinery by the action of a well-characterized proteolysis targeting chimera (PROTAC) molecule. As the major proteolytic machinery, the proteasome system is central to the cell. It is a highly regulated process involved in numerous cellular events and associated with various pathologies. Accordingly, the proteasome is a target for novel therapeutic approaches, and agents targeting the proteasome are already being used in the clinic. The pool of proteins targeted for degradation in a particular cell state may therefore provide important information and open up new therapeutic opportunities. The inventors have developed a strategy based on the method according to the present invention for labelling proteasomes with promiscuous biotin ligases to monitor the interactome of proteasomes in vitro and in vivo. For this purpose, in some embodiments a promiscuous biotin ligase (in the presently shown embodiment BirA* or miniTurbo) is incorporated into fully assembled proteasomes by fusion with a proteasome subunit (in the present example PSMA4) without negatively affecting proteasome activity. These biotin ligases label in situ, i.e., in the cellular context, all proteins that are within their range (proximity). The biotinylated proteins were herein isolated by a known method (Mackmull et al., 2017) using streptavidin beads, and were subsequently digested with protease, and identified by LC-MS/MS. Specific adaptations to the prior art protocol significantly improved the background-to-signal ratio. In addition, the inventors implemented DIA (Data Independent Acquisition) mass spectrometry for label-free analyses. Finally, by using the particularly fast biotin ligase miniTurbo in combination with a known small molecule proteasome inhibitor (MG132), the inventors succeeded in showing that the method can be used for identifying and quantifying endogenous and small molecule-induced enzyme (here proteasome) substrates.

In addition, as a second example a specific mouse model was generated according to an embodiment of the invention that allowed corresponding analyses in vivo. This mouse line constitutively expressed the rTA3 transactivator, thereby enabling doxycycline-inducible expression in all tissues of the proteasome subunit PSMA4 fused to the particularly fast biotin ligase miniTurbo. The inventors applied an embodiment of the present method as an automated high throughput screening platform that can be used to identify molecules suitable for targeted protein degradation (TPD). These are currently of great interest for drug development, as TPD can be used to address therapeutic targets that are otherwise classified as undruggable. Furthermore, the test system according to one embodiment of the present invention is also suitable for the validation of such molecules in vitro and in vivo.

The combination of proximity labelling, and mass spectrometry in the herein applied embodiment of the invention provides a quantitative read out of recruitment of target protein(s) to proteasomes. Since labelling occurred in the cellular context, artifacts due to, e.g., cell lysis were avoided. The use of shot gun mass spectrometry enabled unbiased detection of target recruitment to proteasomes and therefore enabled to reveal off-target effects of the compound analyzed. The approach further enabled to detect targets across a broad range of protein abundance, as demonstrated by the detection of low abundant proteasome substrates such as transcription factors (see FIG. 6). The herein presented approach can generally be applied both in cultured cells and mouse models.

Streptavidin contamination was reduced by acetylation of the lysine residues of streptavidin and additionally by using the protease LysC instead of trypsin for digestion on the beads, thereby significantly improving the signal:background ratio. In addition, one novel combination of features used in the present example was the combination of the particularly fast biotin ligase miniTurbo with a small molecule proteasome inhibitor. Surprisingly, in this way proteasome substrates could be identified for the first time with the present method and the basis for a high-throughput screening method for the identification of new proteasome substrates for drug development could be provided.

### Materials and Methods Used in the Present Examples

The following antibodies were used: anti-FLAG M2 (1:1000, Sigma Aldrich, F3165), Streptavidin HRP (1:40000, Abcam ab7403), anti-PSMA4 (1:250, NOVUS biologicals NBP2-38754), anti-SYNJ1 (1:250, Sigma Aldrich, HPA011916), anti-β-actin (1:5000, Sigma Aldrich, A5441), anti-rabbit HRP-conjugated (1:2000, Dako P0448), anti-mouse HRP-conjugated (1:1500, Dako P0447), anti-FLAG (1:100, Sigma Aldrich, F7425), anti-mouse-Cyanine5 (1:400, Thermo Fisher Scientific, A10524), Streptavidin Alexa Fluor 568 (1:2000, Invitrogen, S11226), anti-Proteasome 20S alpha 1+2+3+5+6+7 (1:200, Abcam, ab22674).

Mice: Rosa26 mice (B6.Cg-Col1a1tm1(tetO-cDNA:Psma4)Mirim/J; B6.Cg-Gt(ROSA)26Sortm2 (CAG-rtTA3,-mKate2)Slowe/J) (Dow et al., 2014) were generated by Mirimus Inc. (NY, USA). All animals were housed (2-5 mice per cage) at the Leibniz Institute on Aging—Fritz Lipmann Institute, in environmentally controlled, pathogen-free animal facility with a 12 hours light/12 hours dark cycle and fed ad libitum with a standard chow or with the doxycycline containing food. Animals used for the procedure were 2-4 months old. Biotin (24 mg/kg body weight) or PBS were administered subcutaneously, daily for 7 consecutive days. At the end of the regime, mice were euthanized with CO2 in a CO2 chamber (VetTech Solutions Ltd., AN045) and the organs isolated using scissors (FST, 14090-09) and forceps (FST, 11018-12). Isolated tissues to be used for mass spectrometry analysis were washed in PBS, weighted, snap-frozen in liquid nitrogen and stored at −80° C. Isolated tissues to be used for immunofluorescence analysis were fixed in 4% formaldehyde, and embedded in paraffin for sectioning using a HistoCore Arcadia H and C (Leica). 4 μm sections were cut using a microm HM 340E (Thermo Fisher) and placed on microscope slides (Menzel, 041300). For subsequent immunofluorescence staining, sections were rehydrated through graded alcohols using a Autostainer XL (Leica) by 2 washes for 10 minutes in xylene followed by 2 washes in 100% ethanol for 3 minutes and 1 minute each, 1 minute wash in 95% ethanol, 1 minute wash in 70% ethanol and 50% ethanol (all v/v in water). Next, slides were washed with PBS and following this usual protocol for immunofluorescence was used.

All the procedures were conducted with a protocol approved by animal experiment license NTP-ID 00040377-1-5 (FLI-20-010) in accordance with the guidelines of the 2010/63 EU directive as well as the instructions of GV SOLAS society.

Cell culture and treatments: Flpln T-REx 293 cells (Thermo Fisher Scientific, R78007), referred to as HEK293T, expressing PSMA4-BirA*, PSMC2-BirA*, BirA*-PSMD3, SYNJ1-BirA*, BirA*-SYNJ1, BirA*, PSMA4-miniTurbo or miniTurbo were generated as described in (Mackmull et al, 2017). Cells were grown in Dulbecco's modified Eagle's medium (DMEM) high glucose 4.5 g/I supplemented with 10% (v/v) heat inactivated fetal bovine serum (FBS), 2 mM L-Glutamine, 15 μg/ml Blasticidin and 100 μg/ml Hygromycin B. U-2 OS cells (ATCC, HTB-96; a kind gift from Pospiech lab, Leibniz Institute on Aging-Fritz Lipmann Institute) were grown in the same cell culture medium as Flpln HEK293 T-REx cells, without antibiotics. All the cells were grown at 37° C., 5% CO2 and 95% humidity in a CO2 incubator. The parental Flpln T-REx 293 cell line was grown in presence of 100 μg/ml Zeocin and 15 μg/ml Blasticidin. Upon generation of stable cell lines, Zeocin™ was replaced by 100 μg/ml Hygromycin B.

For the “BioID” experiments, HEK293T lines were seeded at the density of approximately 1.6×104 cells/cm2 and incubated for 24 hours to allow cell attachment to the culture dish. The expression of BirA* or miniTurbo fusion proteins were induced by a single addition of tetracycline stock (solved in ethanol) exposing the cells to its final concentration of 1 μg/μl in total for 4 days. 2 hours (miniTurbo lines) or 24 hours (BirA* lines) prior to cell harvesting, 50 μM biotin was added to the culture media. For identification of proteasome substrates, PSMA4-miniTurbo or miniTurbo expressing cells were treated with 20 μM MG132 for 4 hours and/or 10 μM KB02-JQ1 for 12 hours. Upon treatment, cells were washed 3×times with PBS and harvested by trypsinization. For each sample, a pellet corresponding to 20 million cells was collected and snap-frozen in liquid nitrogen.

Immunoblot: Cell pellets were lysed in 50 mM HEPES pH 7.5, 5 mM EDTA, 150 mM NaCl, 1% (v/v) Triton X-100, prepared with phosphatase inhibitors and protease inhibitors (both from Roche), for 30 minutes on ice. Lysates were cleared by centrifugation for 15 minutes at 21000×g at 4° C., supernatants transferred to fresh tubes and mixed with loading buffer (1.5 M Tris pH 6.8, 20% SDS (w/v), 85% glycerin (w/v), 5% p-mercaptoethanol (v/v)). This was followed by denaturation for 5 minutes at 95° C. 10-20 μg of sample was loaded on a 4-20% Mini-Protean® TGX™ Gels (BIO-RAD) per lane and separated by SDS-PAGE. Proteins were transferred to a nitrocellulose membrane with a Trans-Blot® Turbo™ Transfer Starter System (BioRad, 170-4155). For high molecular weight samples (SYNJ1-BirA*, and BirA*-SYNJ1) a wet transfer method was used with Hoefer™ TE22 Mini Tank Blotting Unit (Thermo Fisher Scientific, 03-500-216), using a wet transfer buffer (25 mM Tris pH 8.3, 192 mM glycine, 15% (v/v) Methanol). Membranes were stained with PonceauS for 5 minutes on a shaker, washed and imaged on a Molecular Imager ChemiDoc™ XRS+ Imaging system (BioRad) and destained. After incubation for 1 hour in blocking buffer (3% BSA (w/v), 25 mM Tris, 75 mM NaCl, 0.5% (v/v) Tween-20), membranes were incubated overnight at 4° C. with primary antibodies diluted in blocking buffer for FLAG® M2 (1:1000), Streptavidin HRP (1:40000), PSMA4 (1:250), SYNJ1 (1:250), p-actin (1:5000). This was followed by a 1 hour incubation with secondary antibodies dilution matching species conjugated with HRP (1:2000, anti-rabbit; 1:1500, anti-mouse, in 0.3% BSA in TBST (w/v)). Proteins were detected using the enhanced chemiluminescence detection kit (ECL) following the manufacturer instructions. Signals were acquired on the Molecular Imager ChemiDocTMXRS+ Imaging system (BioRad).

The immunoblots for anti-FLAG and Streptavidin-HRP on samples from PSMA4-BirA*, PSMC2-BirA* and BirA*-PSMD3, the cells were lysed in RIPA buffer (150 mM NaCl, 1% Triton X-100 (v/v), 0.5% sodium deoxycholate (w/v), 0.1% SDS (w/v); 50 mM Tris, pH8) prepared with phosphatase inhibitors and protease inhibitors. Samples were incubated on ice for 10 minutes and lysates were prepared by sonication in Bioruptor Plus sonication device (Diagenode). The following steps were performed as indicated previously but using a different buffer to check the efficiency of the proteins to the membrane (amido black solution (0.25% (w/v), naphthol blue black, 45% (v/v) methanol, 10% (v/v) acetic acid, in milliQ water). After the ECL reaction, membranes were visualized on a CL-XPosure Film (Thermo Fisher Scientific, 34090), using an Amersham Hypercassette Autoradiography Cassette (RPN11648).

Proteasome activity assay: The proteasome activity assay (PAA) was performed using the 20S proteasome activity assay kit (Millipore, APT280) following the manufacturer instructions. In short, cell pellets were thawed in ice-cold lysis buffer (50 mM HEPES pH 7.5; 5 mM EDTA; 150 mM NaCl; 1% (v/v) Triton X-100; 2 mM ATP) and left on ice for 30 minutes with short vortex steps (VWR international, VWR/444-1372) every 10 minutes. Samples were centrifuged at 20817×g, for 15 minutes at 4° C. to remove any debris. For protein amount estimation the EZQ Protein Quantitation Kit (Invitrogen, R33200) was used. 50 μg of protein extract were incubated with fluorophore-linked peptide substrate (LLVY-7-amino-4-methylcoumarin, AMC) for 60 minutes at 37° C. Proteasome activity was measured by quantification of fluorescent units from cleaved AMC at 380/460 nm using a microplate reader m1000 (Tecan).

Size-exclusion chromatography (SEC): Pellets of 80 million HEK293T cells expressing PSMA4-BirA* were collected and snap frozen in liquid nitrogen. The pellets were resuspended in 2 ml lysis buffer (50 mM HEPES pH 6.8; 1 mM MgCl2; 1 mM DTT; 20 mM NaCl; 5% glycerol; phosphatase inhibitors and protease inhibitors) and incubated 30 minutes on ice. Cell swelling and lysis was checked in 15 minutes intervals. Cell lysis was assisted by passage of the sample through a 27 G needle 12 times. Following this, the final concentration of NaCl was adjusted to 150 mM. The samples were then clarified by subsequent centrifugation steps as follows: (i) 500×g for 5 minutes at 4° C., (ii) 1000×g for 13 minutes at 4° C., and (iii) 100000×g for 30 minutes at 4° C. The final supernatant was concentrated using 30 kDa cut-off spin filters (Merck Amicon Ultra −0.5 ml, centrifugal filters, UFC503096) to a final protein concentration of approximately 10 μg/μl measured by OD280, and further applied to size-exclusion chromatography.

SEC was performed using an AKTA avant (GE Akta avant 25-1) system equipped with UV detection at 280 nm wavelength. A Yarra-SEC-4000 column (300×7.8 mm, pore size 500 Å, particle size 3 μm) was used with a SecurityGard cartridge GFC4000 4×3.0 mm ID as a guard column. Running conditions were 4° C., a flow rate of 0.5 ml/minutes and run time of 40 minutes. The mobile phase contained 50 mM HEPES, pH 6.8, 1 mM MgCl2, 1 mM DTT, 150 mM NaCl, and 5 mM ATP. A control sample (Phenomenex) was injected prior to each sample to verify column performance. 100 μl samples from 10 mg/ml lysate solution were injected, corresponding to 1 mg protein extract on column. Fractions (200 μl each) were collected along with the LC separation directly in the SDS buffer, to a final concentration of 4%. Thirty-six fractions were further processed for LC-MS/MS analysis. Of these 36 fractions the first and last two fractions were pooled.

Preparation of SEC fractions for mass spectrometry analysis: The SEC fractions were further processed by addition of DTT (50 mM) in 100 mM HEPES at pH 8, boiled for 5 minutes at 95° C., followed by sonication (Diagenode Bioruptor Plus) for 10 cycles (30 s on/60 s off) at 4° C. The samples were then centrifuged at 3000×g for 5 minutes at room temperature, and the supernatant transferred to 2 ml tube. This was followed by alkylation with 20 mM iodoacetamide (IAA) for 30 minutes at room temperature in the dark. Protein amounts were confirmed by SDS-PAGE (4%). Protein samples in the collected fractions ranged from 10-100 μg. Proteins were precipitated overnight at −20° C. after addition of a 4×volume of ice-cold acetone. Thereafter, the samples were centrifuged at 20800×g for 30 minutes at 4° C. and the supernatant carefully removed. Pellets were washed twice with 1 ml ice-cold 80% (v/v) acetone and then centrifuged with 20800×g at 4° C. The samples were air-dried before addition of 120 μl of digestion buffer (3 M urea; 100 mM HEPES, pH8). Samples were resuspended by sonication (as above) and LysC was added at 1:100 (w/w) enzyme:protein ratio. The samples were then digested for 4 hours at 37° C. (1000×rpm for 1 hour, then 650×rpm, Eppendorf ThermoMixerC). Samples were then diluted 1:1 with milliQ water, and trypsin added at the same enzyme to protein ratio. Samples were further digested overnight at 37° C. (650×rpm). Consequently, digests were acidified by the addition of TFA to a final concentration of 2% (v/v) and then desalted with Waters Oasis (HLB μElution Plate 30 μm, Waters Corporation, Milford, MA, USA) with slow vacuum. Therefore, the columns were conditioned three times with 100 μl solvent B (80% (v/v) acetonitrile; 0.05% (v/v) formic acid and equilibrated three times with 100 μl solvent A (0.05% (v/v) formic acid in Milli-Q water). The samples were loaded, washed 3 times with 100 μl solvent A, and then eluted with 50 μl solvent B. The eluates were dried in a vacuum concentrator.

### BioID Affinity Purification

Cell pellets were thawed on ice were resuspended in 4.75 ml of BioID lysis buffer (50 mM Tris pH 7.5; 150 mM NaCl; 1 mM EDTA; 1 mM EGTA; 1% (v/v) Triton X-100; 1 mg/ml aprotinin; 0.5 mg/ml leupeptin; 250 U turbonuclease; 0.1% (w/v) SDS), followed by 1 hour incubation in the rotator mixer (STARLAB RM Multi-1) (15×rpm) at 4° C. to aid the lysis. Samples were then briefly sonicated in a Bioruptor Plus for 5 cycles (30 s ON/30 s OFF) at high setting and afterwards centrifuged at 20817×g, for 30 minutes at 4° C. to remove any debris.

Mouse organs were thawed and transferred into Precellys lysing kit tubes (Keramik-kit 1.4/2.8 mm, 2 ml (CKM)) containing 1 ml of PBS supplemented with 1 tab of cOmplete, Mini, EDTA-free Protease Inhibitor per 50 ml. For homogenization, organs were shaken twice at 6000×rpm for 30 s using Precellys 24 Dual (Bertin Instruments, Montigny-le-Bretonneux, France), centrifuged at 946×g at 4° C. for 5 minutes, and the resulting homogenate was transferred to a new tube. Based on the estimated protein content (5% of fresh tissue weight for liver and brain, 8% for heart and kidney and 20% for muscle), homogenates corresponding to 4 mg protein were processed for further BioID affinity purification. This entailed cell lysis of the homogenates by means of BioID lysis buffer.

Streptavidin coated Sepharose beads were acetylated by two successive treatments with 10 mM Sulfo-NHS-Acetate for 30 minutes at room temperature. The reaction was then quenched with 1 M Tris pH 7.5 (1:10 v/v) and then the beads were washed with 1×PBS and centrifuged at 2000×g for 1 minute at room temperature. This washing was performed 3 times in total. Cleared lysates were transferred to new tubes, 50 μl of acetylated beads added, and samples were incubated for 3 hours on the rotator (15×rpm) at 4° C. This was followed by centrifugation at 2000×g for 5 minutes at 4° C. and removal of 4.5 ml of the supernatant from each sample. Remaining sample with the beads at the bottom was transferred to a Pierce Spin Column Snap Cap column (Thermo Fisher Scientific, 69725) and the tubes were additionally rinsed with lysis buffer and added to the Pierce Spin Column Snap Cap column. Beads were then washed on the column with a lysis buffer, followed by 3 washes with freshly prepared 50 mM ammonium bicarbonate (AmBic), pH 8.3. The bottom of the columns were closed with a plug and beads transferred to fresh 2 ml tubes by means of 3×300 μl 50 mM AmBic, pH 8.3. The samples were then centrifuged at 2000×g for 5 minutes at 4° C. and the content of each tube was removed until around 200 μl of the liquid (including the bead) remained at the bottom. 1 μg of LysC was added and incubated at 37° C. for 16 hours shaking at 500×rpm. The samples were then centrifuged at 2000×g for 5 minutes at room temperature and the content of the tubes were transferred to Pierce Spin Column Snap Cap columns. The digested peptides were eluted with two times 150 μl of freshly made 50 mM AmBic. To elute biotinylated peptides still bound to the beads, 150 μl of 80% ACN and 20% TFA was added, briefly mixed, and rapidly eluted. This elution step was repeated twice, and the eluates merged. Following elution, 0.5 μg of trypsin was added to the AmBic elutions and digestion continued for an additional 3 hours with mixing at 500×rpm and 37° C. Digested AmBic elutions were then dried down in a vacuum concentrator, resuspended in 200 μl 0.05% (v/v) formic acid in milliQ water and sonicated in a Bioruptor Plus (5 cycles with 1 minute ON and 30 s OFF with high intensity at 20° C.). ACN/TFA elutions were dried down in a vacuum concentrator (not to completeness but until approximately 50 μl were left), 50 μl of 200 mM HEPES pH 8.0 were added to the samples and pH adjusted to 7-9. 0.5 μg of trypsin were then added and digestion continued for an additional 3 hours with mixing at 500×rpm and 37° C. Digested peptides were acidified with 10% (v/v) trifluoroacetic to pH<3. Both AmBic and ACN/TFA elutions were desalted using Macro Spin Column C18 columns following manufacturer's instructions and dried down in a vacuum concentrator.

Preparation of whole proteome samples from mouse organs for mass spectrometry analysis. Mouse organ lysates containing 100 μg of total proteins were processed by acetone precipitation and protein digestion as described in “Preparation of SEC fractions for mass spectrometry analysis”.

### LC-MS/MS Data Acquisition

LC (liquid chromatography): Prior to analysis, samples were reconstituted in in MS Buffer (5% acetonitrile, 95% Milli-Q water, with 0.1% formic acid) and spiked with iRT peptides (Biognosys, Switzerland). Peptides were separated in trap/elute mode using the nanoAcquity MClass Ultra-High Performance Liquid Chromatography system (UPLC) or nanoAcquity UPLC system (Waters, Waters Corporation, Milford, MA, USA) equipped with a trapping (nanoAcquity Symmetry C18, 5 μm, 180 μm×20 mm) and an analytical column (nanoAcquity BEH C18, 1.7 μm, 75 μm×250 mm). Solvent A was water and 0.1% formic acid, and solvent B was acetonitrile and 0.1% formic acid. 1 μl of the sample (~1 μg on column) were loaded with a constant flow of solvent A at 5 μl/minutes onto the trapping column. Trapping time was 6 minutes. Peptides were eluted via the analytical column with a constant flow of 0.3 μl/minutes. During the elution, the percentage of solvent B increased in a nonlinear fashion from 0-40% in 90 min (120 minutes for total proteome of mouse organs). Total run time was 115 minutes (145 minutes) including equilibration and conditioning. The LC was coupled to an Orbitrap Fusion Lumos (Thermo Fisher Scientific, Bremen, Germany) using the Proxeon nanospray source or to an Orbitrap Q-Exactive HFX (Thermo Fisher Scientific) for BioID experiments from HEK293T cells, or to an Orbitrap Exploris 480 (Thermo Fisher Scientific, Bremen, Germany) for BioID experiments combined with PROTAC treatment. The peptides were introduced into the mass spectrometer via a Pico-Tip Emitter 360-μm outer diameter×20-μm inner diameter, 10-μm tip (New Objective) heated at 300° C., and a spray voltage of 2.2 kV was applied. For data acquisition and processing of the raw data Tune version 2.1 and Xcalibur 4.1 (Orbitrap Fusion Lumos), Tune 2.9 and Xcalibur 4.0 (Orbitrap Q-Exactive HFX) and Tune 3.1 and Xcalibur 4.4 (Orbitrap Exploris 480) were employed.

DDA (Data-dependent acquisition): SEC fractions, mouse BioID as well as BioID of PSMA4, PSMC2 and PSMD3 were analyzed using DpD (DDA plus DIA). Here, data from a subset of conditions were first acquired in DDA mode to contribute to a sample specific spectral library. Full scan MS spectra with mass range 375-1500 m/z (using quadrupole isolation) were acquired in profile mode in the Orbitrap with resolution of 60,000 FWHM. The filling time was set at a maximum of 50 ms with a limitation of 2×105 ions. The “Top Speed” method was employed to take the maximum number of precursor ions (with an intensity threshold of 5×105) from the full scan MS for fragmentation (using HCD collision energy, 30%) and quadrupole isolation (1.4 Da window) and measurement in the Orbitrap, with a cycle time of 3 seconds. The MIPS (monoisotopic precursor selection) peptide algorithm was employed. MS/MS data were acquired in centroid mode in the Orbitrap, with a resolution of 15,000 FWHM and a fixed first mass of 120 m/z. The filling time was set at a maximum of 22 ms with a limitation of 1×105 ions. Only multiply charged (2+−7+) precursor ions were selected for MS/MS. Dynamic exclusion was employed with maximum retention period of 15 s and relative mass window of 10 ppm. Isotopes were excluded.

The DIA (Data-dependent acquisition) data acquisition was the same for both directDIA and DpD. Full scan mass spectrometry (MS) spectra with mass range 350-1650 m/z were acquired in profile mode in the Orbitrap with resolution of 120,000 FWHM. The default charge state was set to 3+. The filling time was set at a maximum of 60 ms with a limitation of 3×106 ions. DIA scans were acquired with 34 mass window segments of differing widths across the MS1 mass range. Higher collisional dissociation fragmentation (stepped normalized collision energy; 25, 27.5, and 30%) was applied and MS/MS spectra were acquired with a resolution of 30,000 FWHM with a fixed first mass of 200 m/z after accumulation of 3×106 ions or after filling time of 35 ms (whichever occurred first). Data were acquired in profile mode.

LC-MS/MS data analysis: DpD (DDA plus DIA) libraries were created by searching both the DDA runs and the DIA runs using Spectronaut Pulsar (v 13-15, Biognosys, Zurich, Switzerland). The data were searched against species specific protein databases (Homo sapiens, reviewed entry only (16,747 entries), release 2016_01 or Mus musculus, entry only (20,186), release 2016_01 respectively) with a list of common contaminants appended. The data were searched with the following modifications: carbamidomethyl (C) as fixed modification, and oxidation (M), acetyl (protein N-term), and biotin (K) as variable modifications. A maximum of 2 missed cleavages was allowed. The library search was set to 1% false discovery rate (FDR) at both protein and peptide levels. This library contained 79,732 precursors, corresponding to 4,730 protein groups for SEC fractions, 77,401 precursors, corresponding to 5,125 protein groups for BioID on PSMA4 and PSMC2 and 67,490 precursors, corresponding to 4,525 protein groups for mouse BioID using Spectronaut protein inference. All other BioID and mouse proteome experiments were processed using the directDIA pipeline in Spectronaut Professional (v. 13-15). The data were searched against a species specific (Mus musculus and Homo sapiens, as described above) with a list of common contaminants appended. BGS factory settings were used with the exception of: variable modifications=acetyl (protein N-term), biotin (K), oxidation (M).

SEC-MS experiments were processed using Spectronaut v. 13 with default settings except: Proteotypicity Filter=Only Protein Group Specific; Major Group Quantity=Median peptide quantity; Major Group Top N=OFF; Minor Group Quantity=Median precursor quantity; Minor Group Top N=OFF; Data Filtering=Qvalue sparse; Imputing Strategy=No imputing; Cross run normalization=OFF.

PSMA4, PSMC2 and PSMD3 BioID experiments were processed using Spectronaut v. 13-17 with default settings except: Proteotypicity Filter=Only Protein Group Specific; Major Group Quantity=Median peptide quantity; Major Group Top N=OFF; Minor Group Quantity=Median precursor quantity; Minor Group Top N=OFF; Data Filtering=Qvalue percentile (0.5); Imputing Strategy=No imputing; Normalization Strategy=Global Normalization; Normalize on=Median; Row Selection=Qvalue sparse.

SYNJ1 BioID experiments were processed using Spectronaut v. 13 with default settings except: Proteotypicity Filter=Only Protein Group Specific; Major Group Quantity=Median peptide quantity; Major Group Top N=OFF; Minor Group Quantity=Median precursor quantity; Minor Group Top N=OFF; Data Filtering=Qvalue percentile (0.5); Imputing Strategy=No imputing; Normalization Strategy=Global Normalization; Normalize on=Median; Row Selection=Qvalue complete.

Mouse BioID and BioID experiments combined with PROTAC treatment were processed using Spectronaut v. 15 with default settings except: Proteotypicity Filter=Only Protein Group Specific; Major Group Quantity=Median peptide quantity; Major Group Top N=OFF; Minor Group Quantity=Median precursor quantity; Minor Group Top N=OFF; Data Filtering=Qvalue percentile (0.2); Imputing Strategy=Global imputing; Normalization Strategy=Global Normalization; Normalize on=Median; Row Selection=Qvalue complete.

Mouse proteome data were processed using Spectronaut v. 15 with default settings except: Proteotypicity Filter=Only Protein Group Specific; Normalization Strategy=Local Normalization.

For all the BioID experiments, differential abundance testing was performed in Spectronaut using a paired t-test between replicates. P values were corrected for multiple testing multiple testing correction with the method described by Storey (Storey, 2002). The candidates and protein report tables were exported from Spectronaut and used for volcano plots generation and Principal Component Analysis (PCA), respectively, using R and RStudio server.

Immunofluorescence: Cells were grown on coverslips (Carl Roth, YX03.1) in 12-well plates (Lab solute, 7696791), 25000 cells per well. Cells were washed three times with 1×PBS, fixed in 4% formaldehyde (v/v) in PBS for 10 minutes at room temperature, washed 3×5 minutes with 1×PBS and permeabilized with permeabilization buffer (0.7% Triton X-100; in 1×PBS) at room temperature for 15 minutes. Washing with PBS was repeated 2×5 minutes and samples were incubated with blocking solution (10% (w/v) BSA; 10% (v/v) Triton X-100; 5% (v/v) goat serum) for 10 minutes at room temperature. The coverslips were incubated with primary antibody anti-FLAGM2 at 4° C. overnight (Sigma Aldrich, mouse IgG1, 1:100). After washing 3×5 minutes with PBS/PBST (first with PBS, second with PBS+0.2% (v/v) Tween 20, third with PBS) the secondary fluorescence-labeled antibody (goat anti-mouse IgG (H+L)—Cyanine5, 1:400 in blocking solution) and fluorescently labeled streptavidin, 1:2000 in blocking solution) were incubated for 30 minutes at 37° C. After 3×5 minutes with PBS/PBST (first with PBS, second with PBS+0.2% (v/v) Tween 20, third with PBS), nuclei were stained with DAPI (4′,6-Diamidino-2-Phenylindole, Dihydrochloride, 0.02 μg/μl in PBS) at room temperature for 10 minutes and washed again with PBS 2×5 minutes. Frozen sections and single fibers were mounted in Permafluor mounting medium using glass slides (041300, Menzel) and dried at room temperature overnight. All samples were stored at 4° C. in the dark until further analysis by microscopy. Immunofluorescence microscopy was performed with an Axio Imager (Z2 using a Plan-Apochromat 20×/0.8 M27 Objective) and analyzed with the software Zen 2 Blue Edition (Carl Zeiss Microscopy GmbH).

Proximity ligation assay (PLA): Cells were grown in 12 well chambers (ibidi, 81201) coated with Poly-D-Lysin, with 5000 cells per well. Cells were washed three times with PBS, fixed in 4% formaldehyde in PBS for 10 minutes at room temperature, washed 3×5 minutes with PBS and permeabilized with permeabilization buffer (0.7% Triton X-100; in PBS) at room temperature for 15 minutes. This was followed by washes of 2×5 minutes with PBS, and samples were incubated with Duolink Blocking Solution (40 μl per well), for 60 minutes at 37° C. in a humidity chamber. The blocking solution was removed, and cells were incubated at 4° C. overnight in a humidity chamber with following primary antibodies: anti-Proteasome 20S alpha 1+2+3+5+6+7 (1:200 in Duolink Antibody Diluent) and anti-SYNJ1 (1:100 in Duolink Antibody Diluent). Primary antibody solutions were removed and the slides with the cells were washed 2×5 minutes in Duolink In Situ wash buffer A at room temperature. This was followed by incubation with PLA probe solution (Duolink In Situ PLA® Probe Anti-Mouse MINUS, diluted 1:5 in Duolink Antibody Diluent and Duolink In Situ PLA® Probe Anti-Rabbit PLUS, diluted 1:5 in Duolink Antibody Diluent) for 1 hour at 37° C.—samples propped up on a rack in a water bath (VWR, VWB18). The cells were washed 2×5 minutes in Duolink In Situ Wash Buffer A at room temperature, followed by incubation with ligase solution in ligation buffer (Ligase from Duolink In Situ Detection Reagents Red kit, diluted 1:40 in ligation buffer from the same kit) for 30 minutes at 37° C. in a water bath. The cells were washed 2×5 minutes in Duolink In Situ Wash BufferA at room temperature, followed by incubation with polymerase solution in amplification buffer (Polymerase from Duolink In Situ Detection Reagents Red kit, diluted 1:80 in diluted 5×Amplification Red buffer from the same kit) for 100 minutes at 37° C. in a water bath. Cells were then washed 2×10 minutes in Duolink In Situ Wash Buffer B at room temperature followed by a wash in 0.01×Duolink In Situ Wash Buffer B for 1 minute at room temperature. The slides with the cells were then mounted with a coverslip (ibidi, 10811) using Duolink In Situ Mounting Medium with DAPI and dried at room temperature overnight. All samples were stored at 4° C. in the dark until further analysis with the microscope. Immunofluorescence microscopy was performed with an Axio Imager Z2 using a Plan-Apochromat 20×/0.8 M27 Objective (Zeiss).

Molecular visualization and structure analysis: For visualization of proteasome complexes UCSF Chimera program (version 1.13.1) was used. The three-dimensional structural data of macromolecular complexes of proteasome were downloaded from the Protein Data Bank (PDB) database (5TOC). For the analysis of the enrichment of proteasome subunits in BioID protocol, data sets with fold change information were used and filtered the following way: q value<0.05, number of identified unique peptides per protein 2. The intensity of the proteasome subunit coloring used was directly dependent on the fold change of the identified subunit in the BioID affinity purification.

### Example 1

### Design of a Proximity Labeling Strategy to Monitor Proteasome Interactions

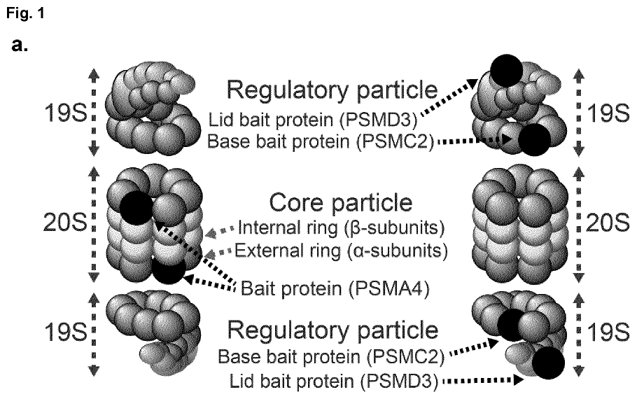

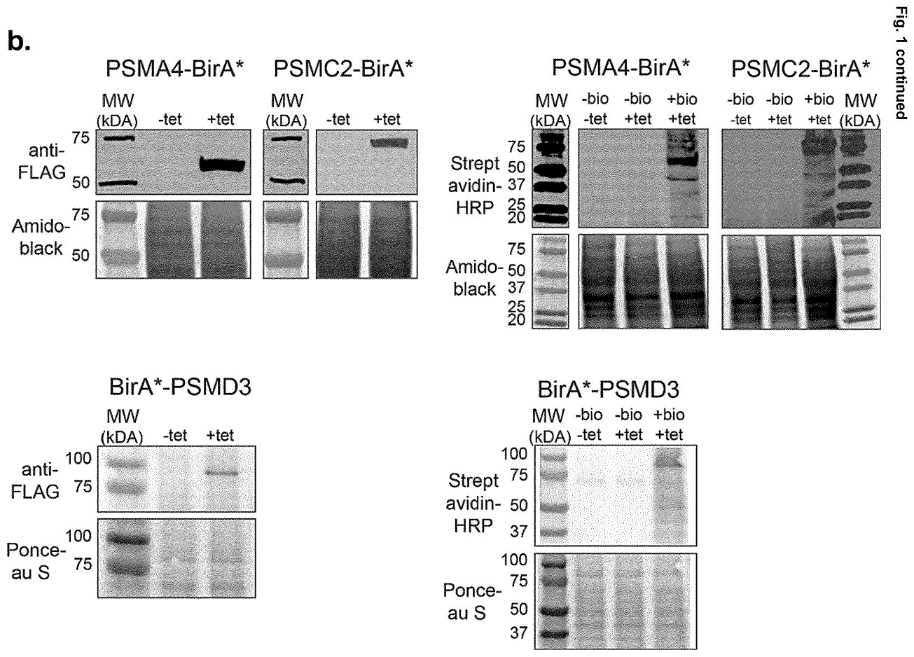

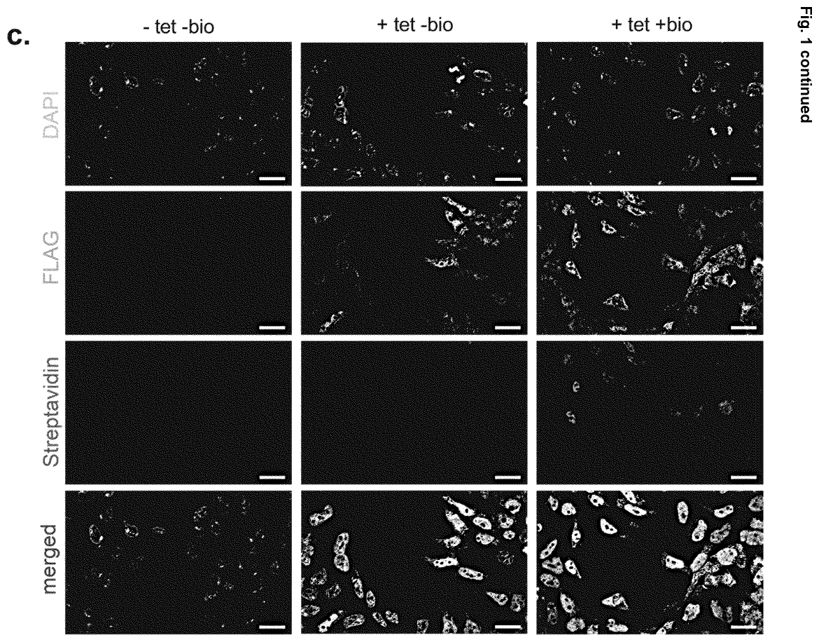

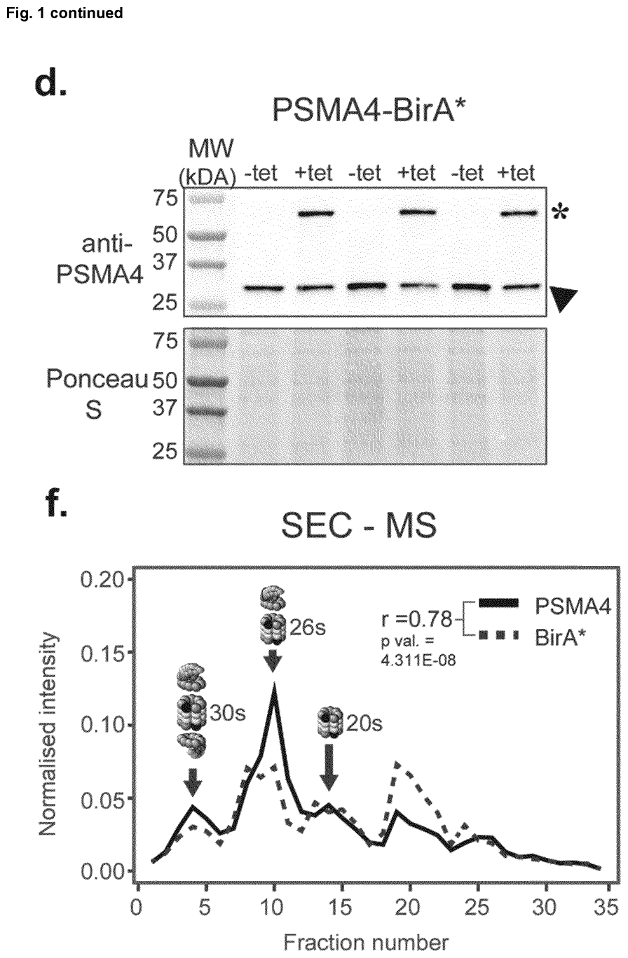

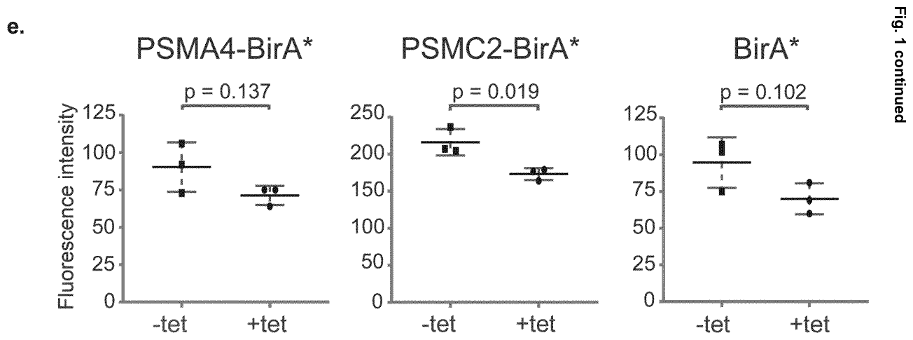

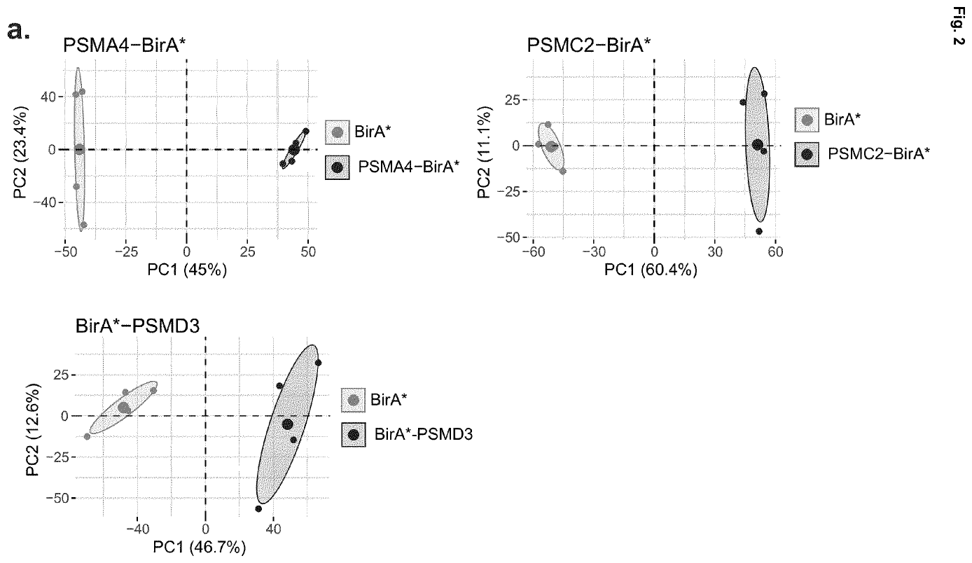

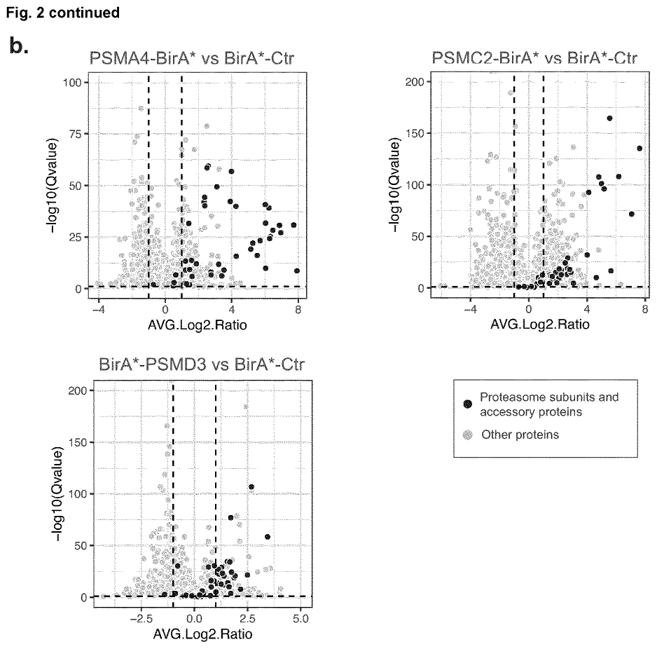

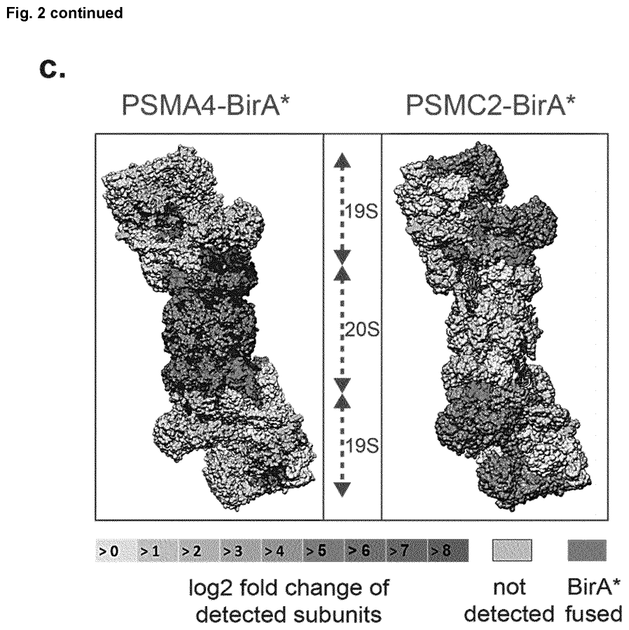

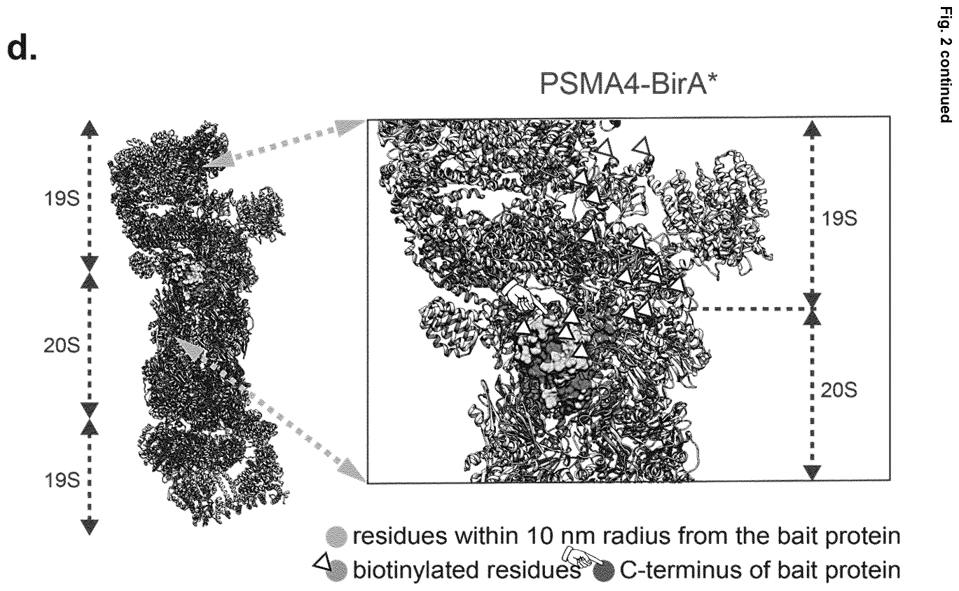

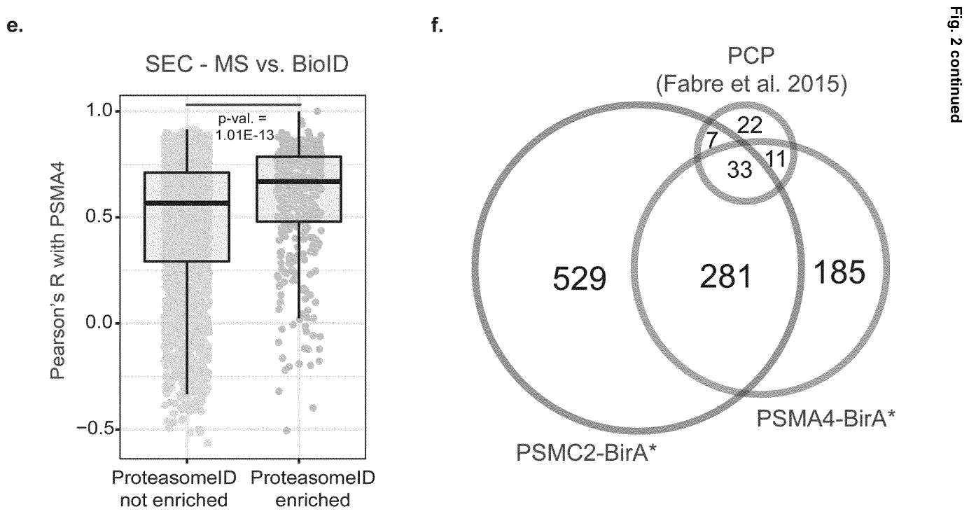

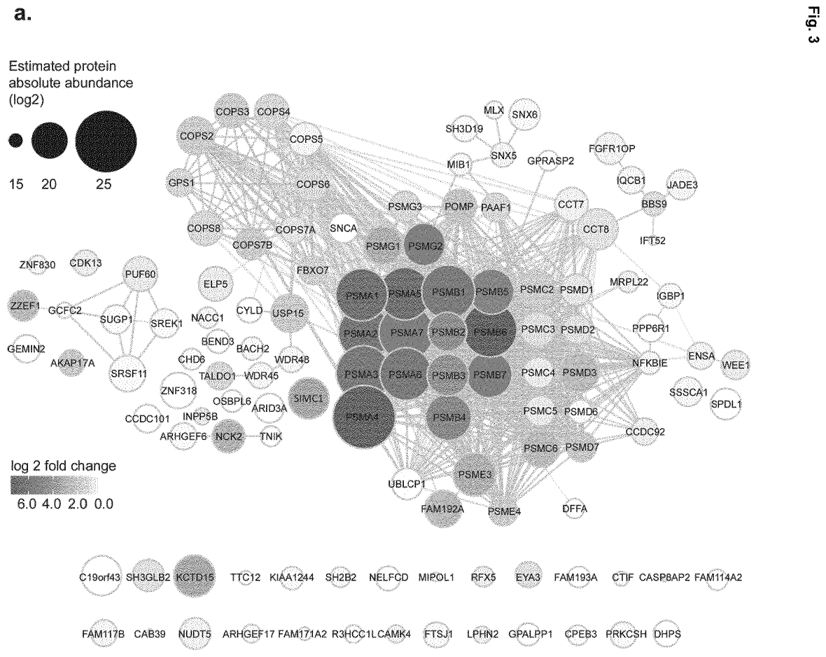

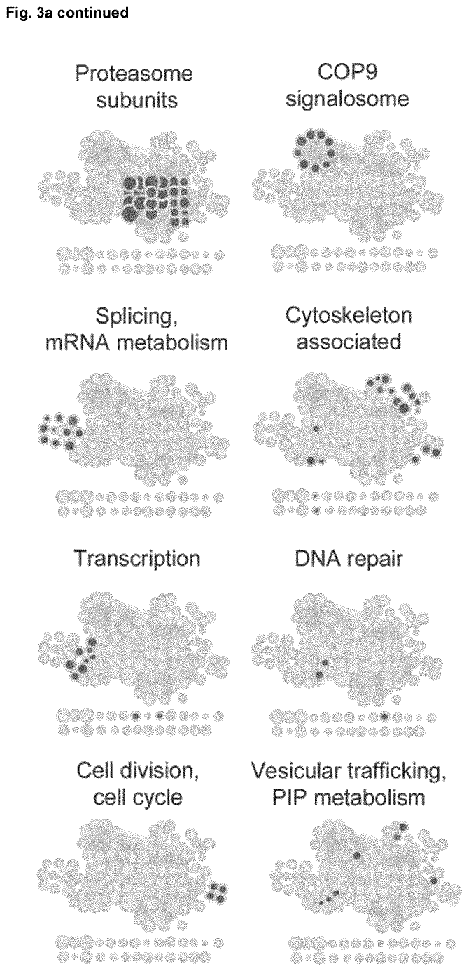

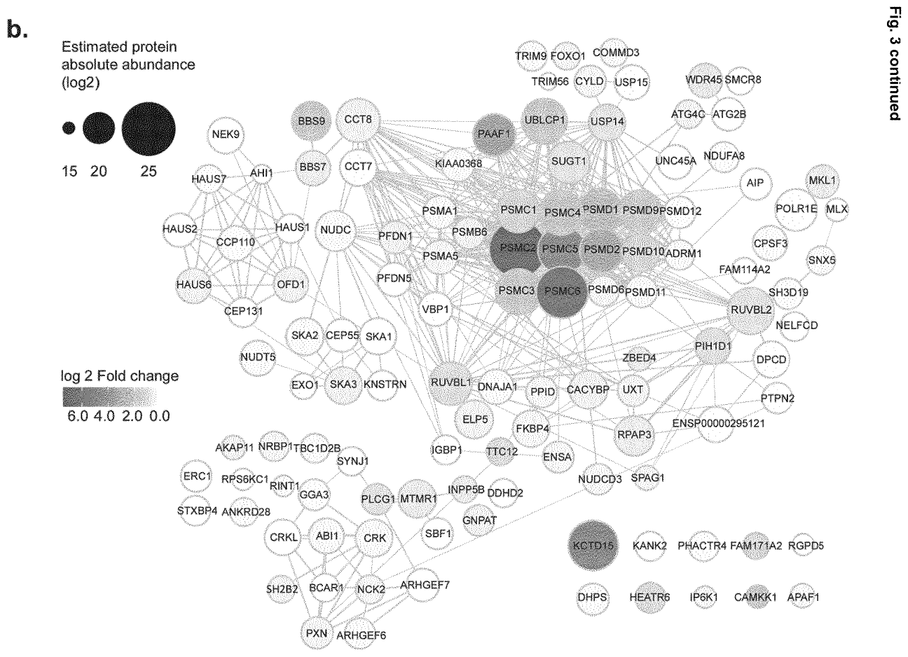

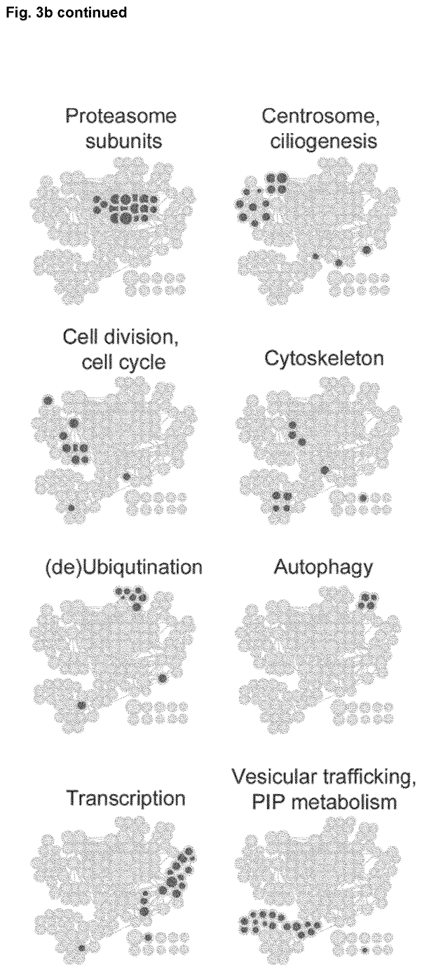

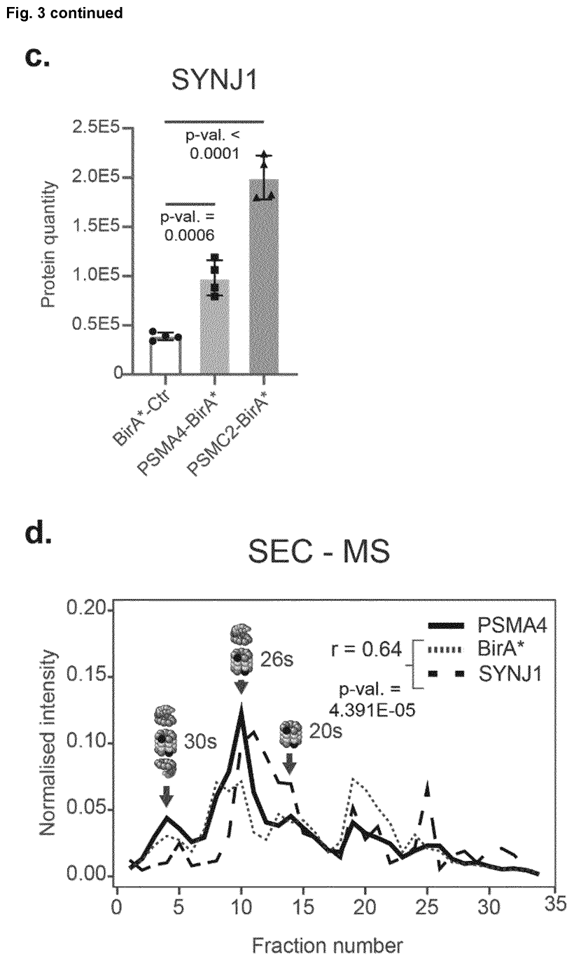

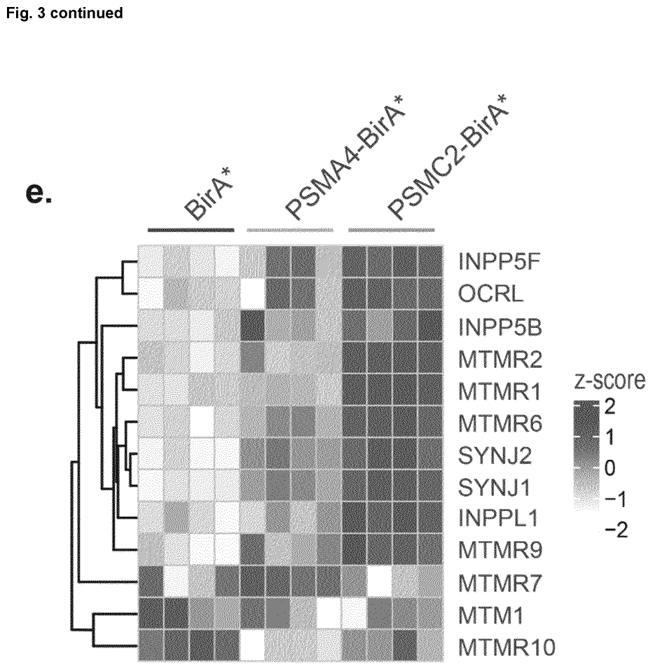

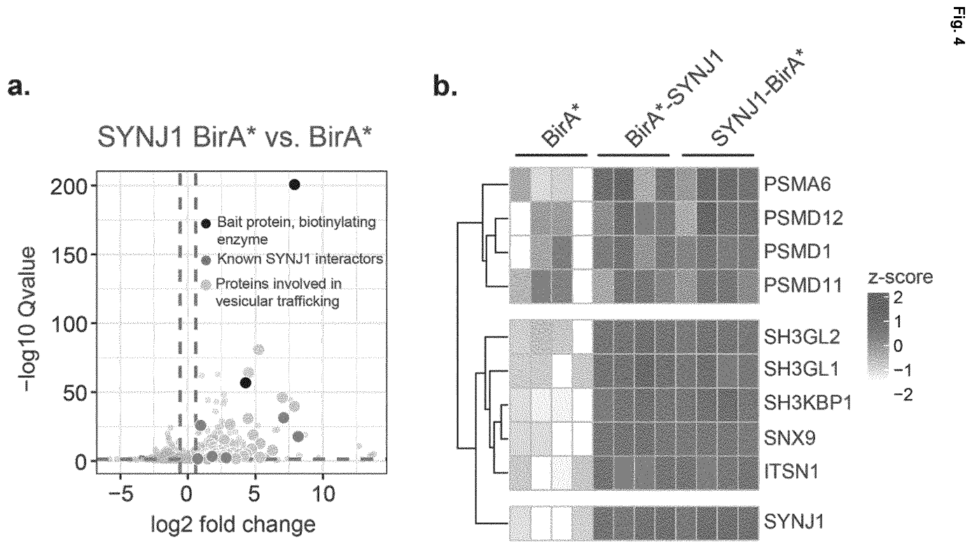

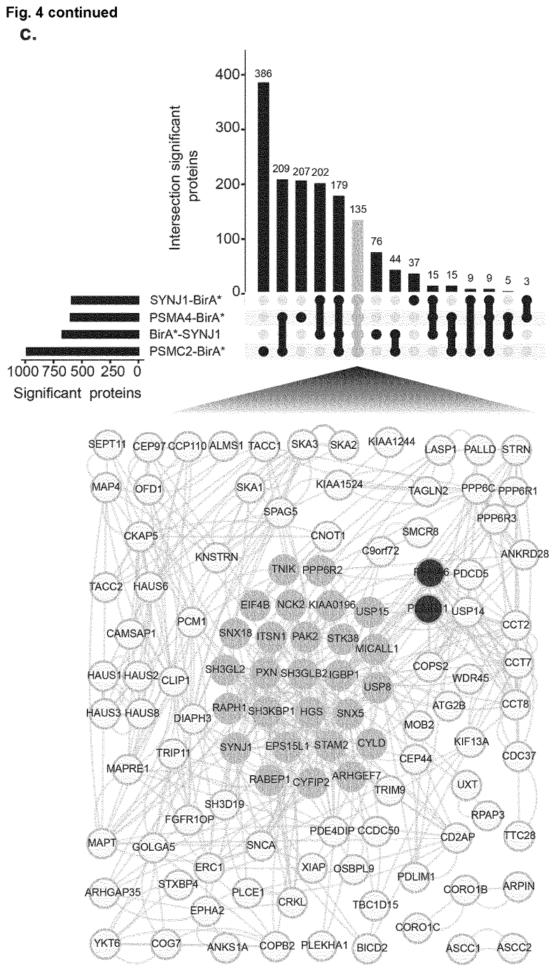

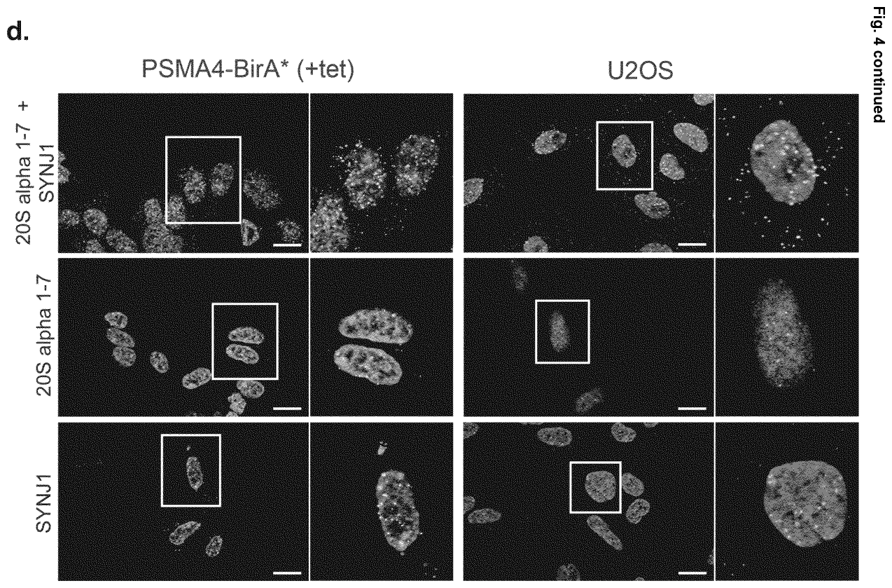

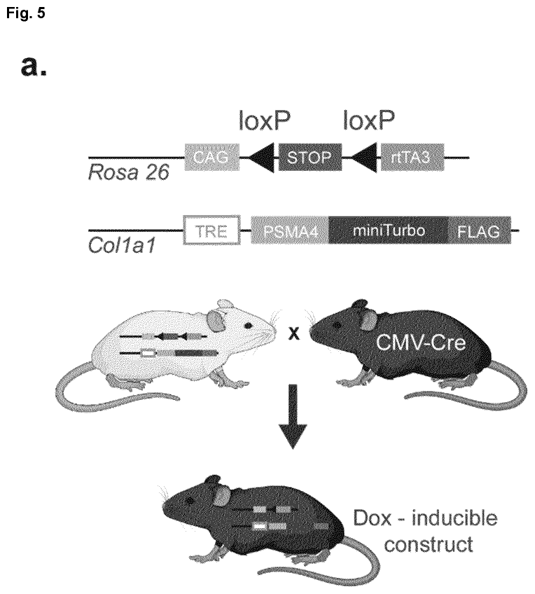

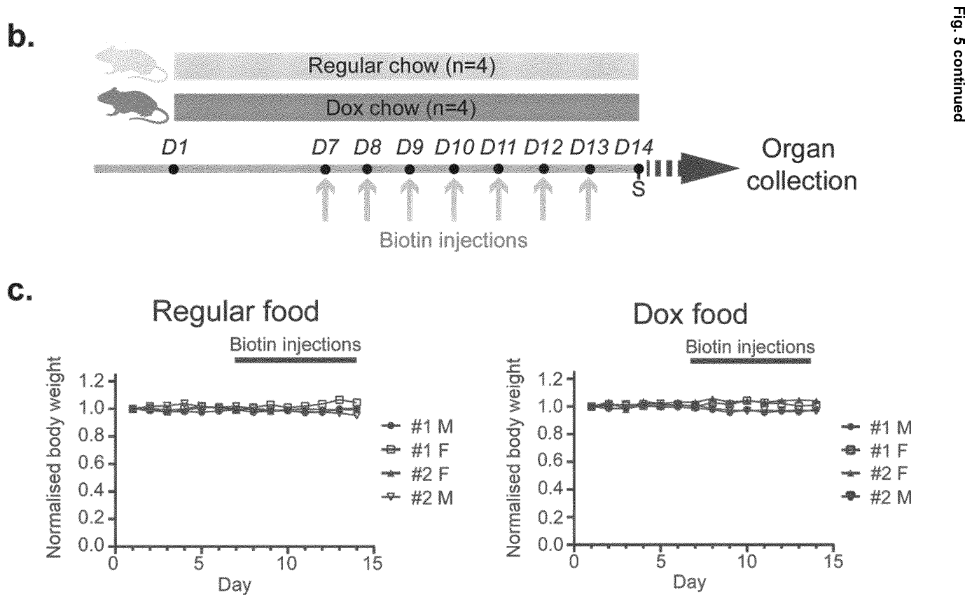

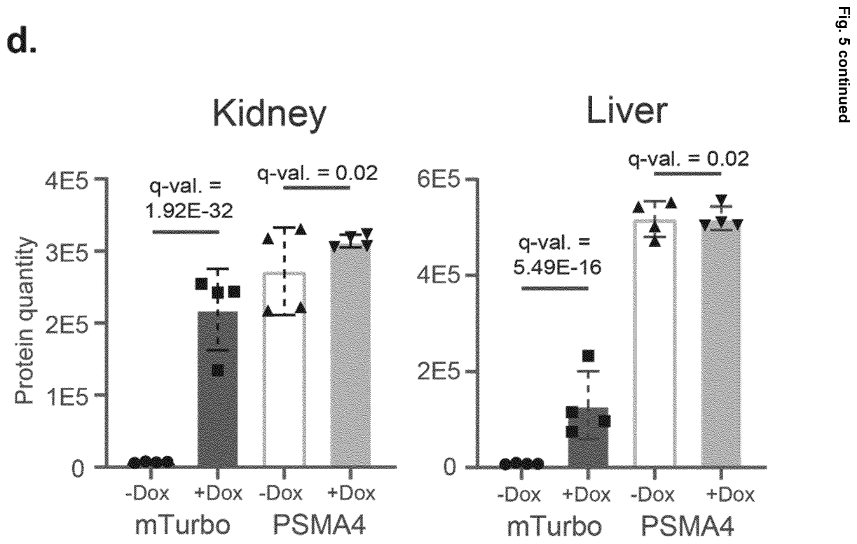

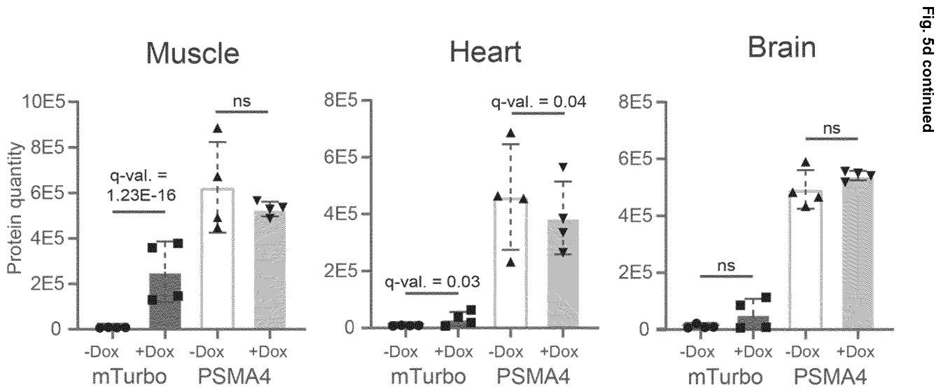

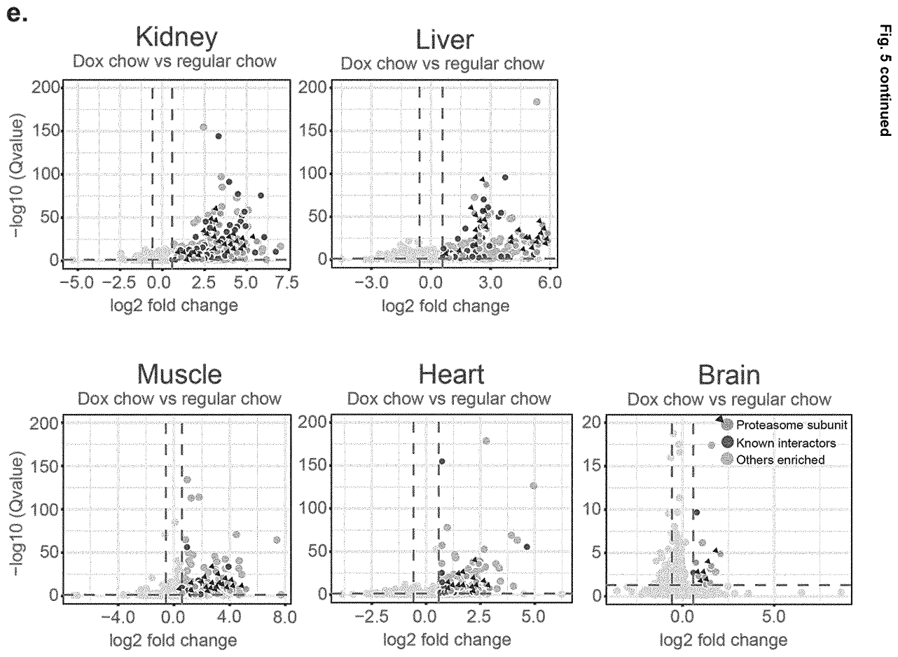

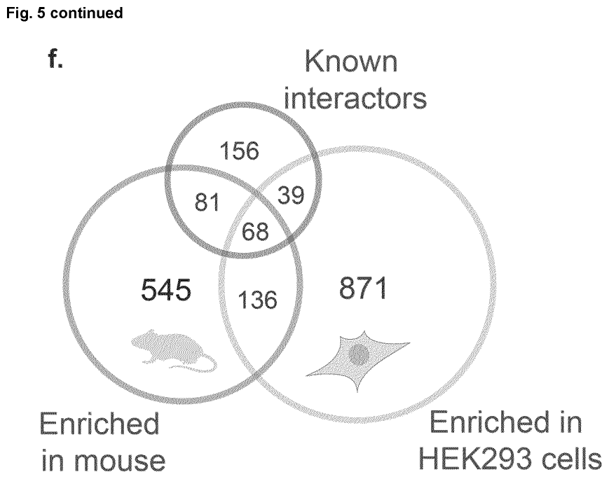

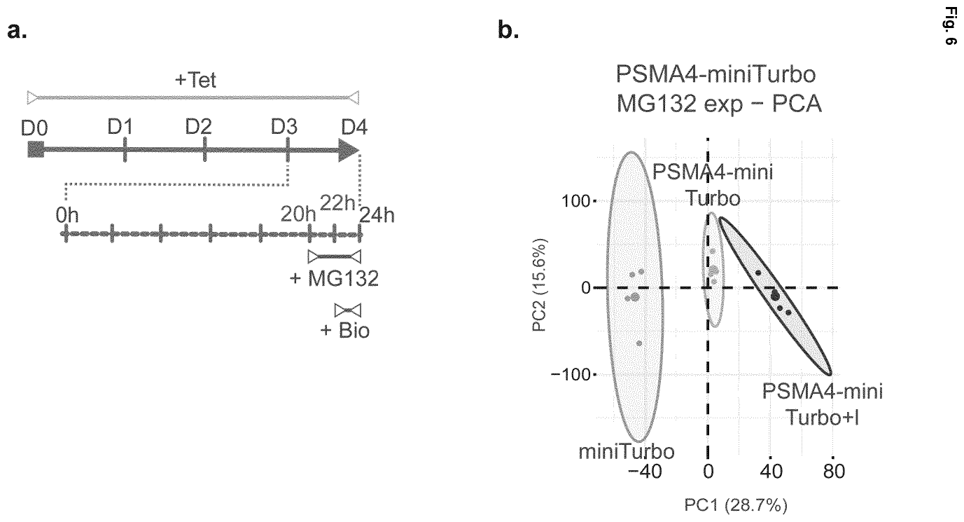

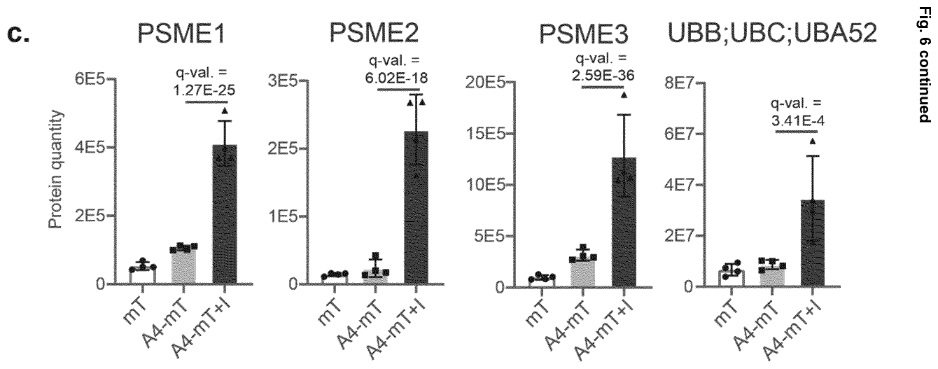

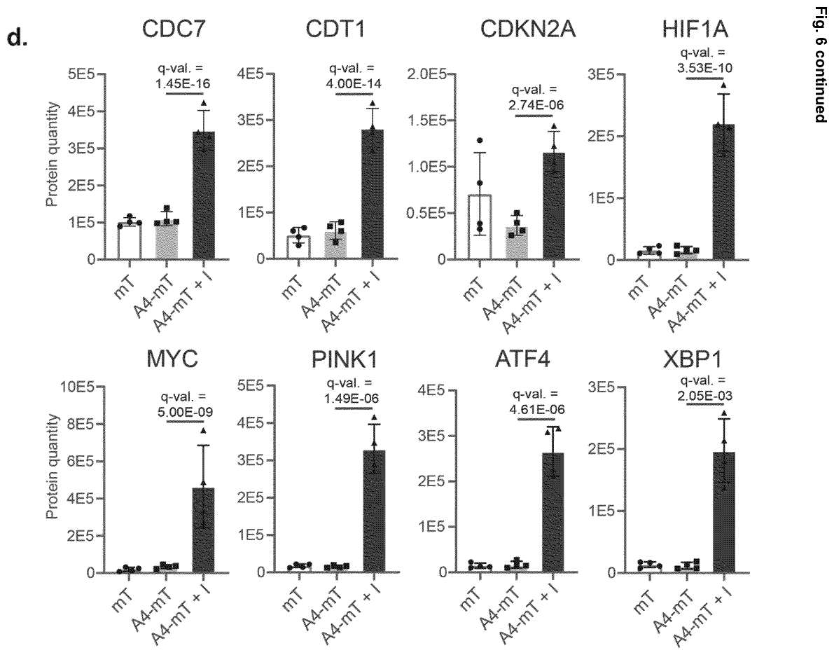

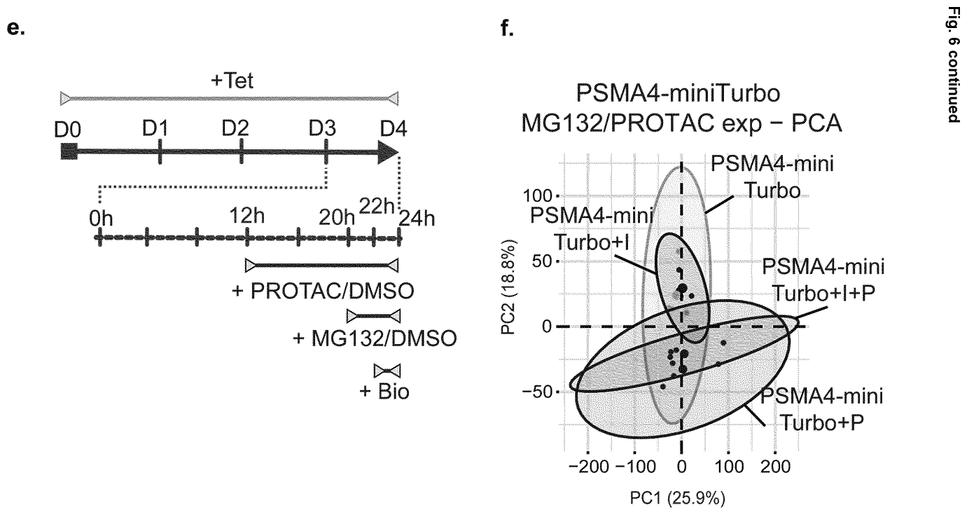

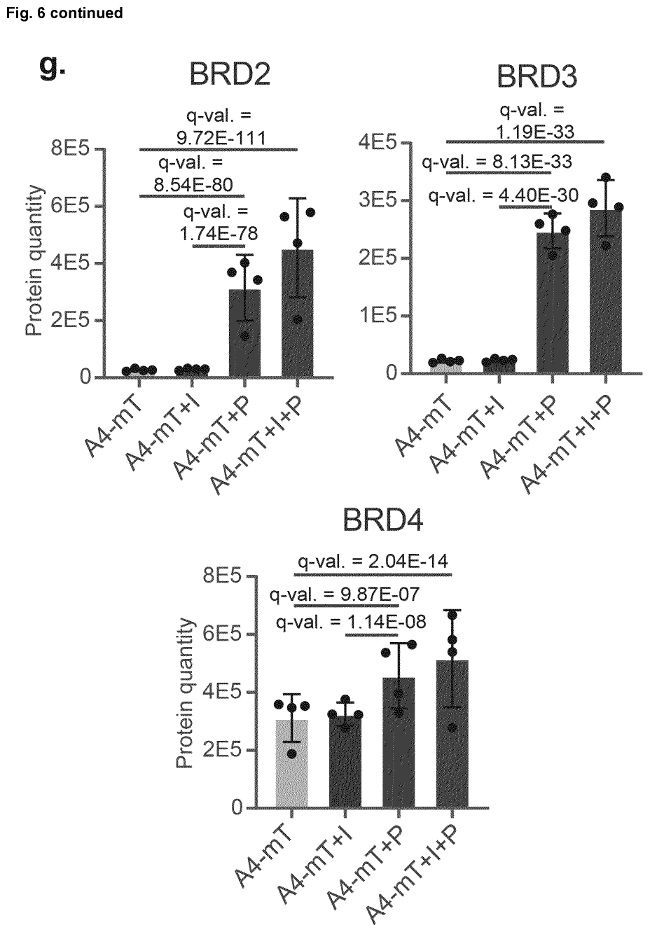

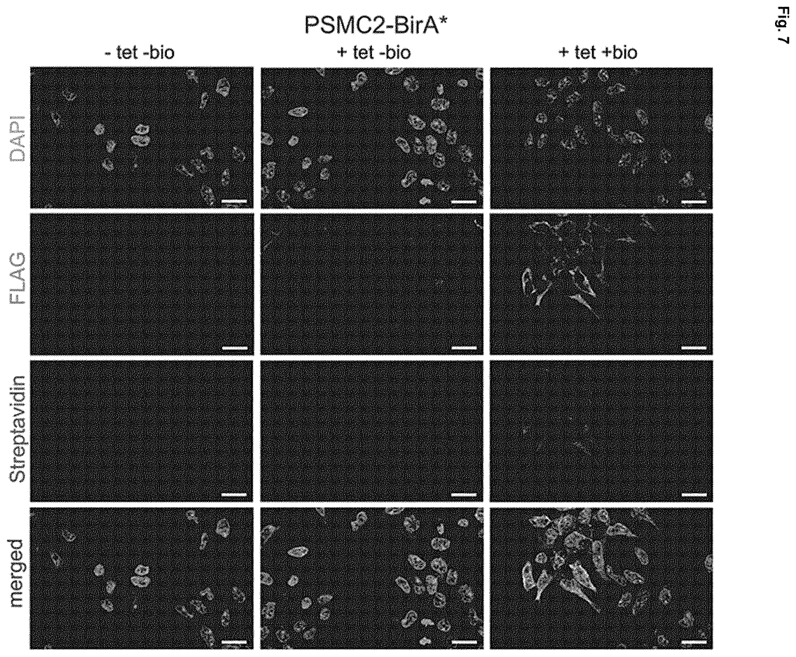

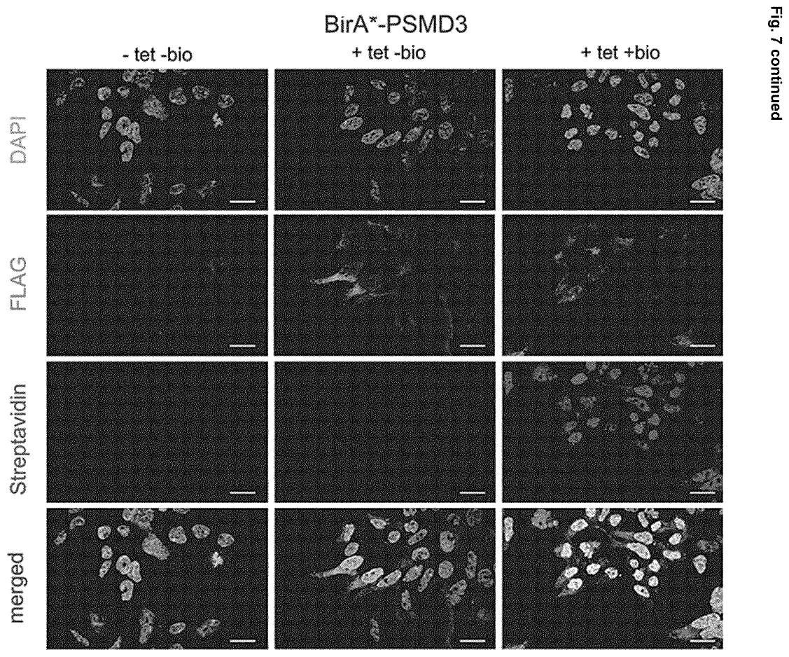

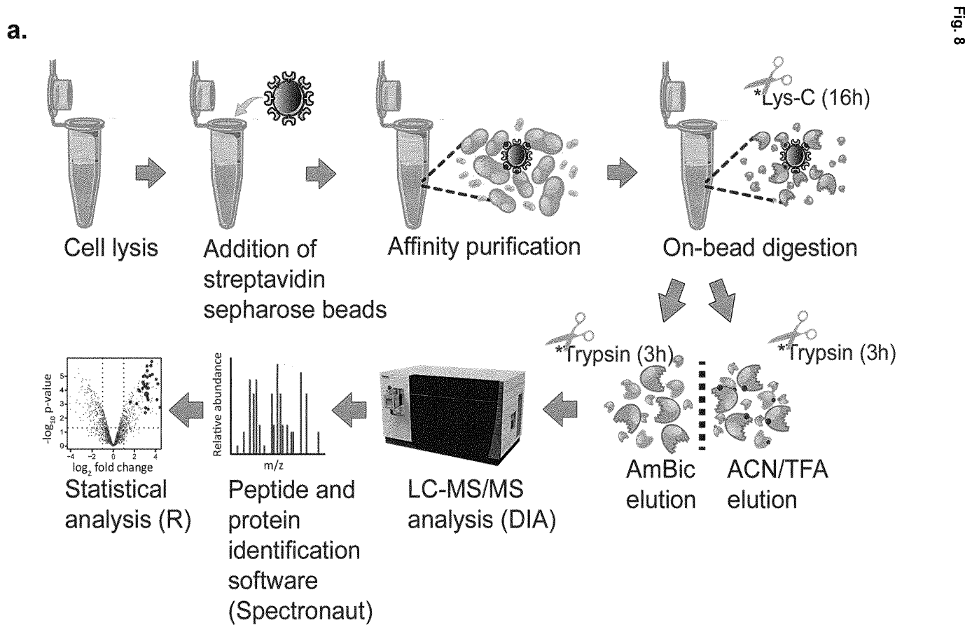

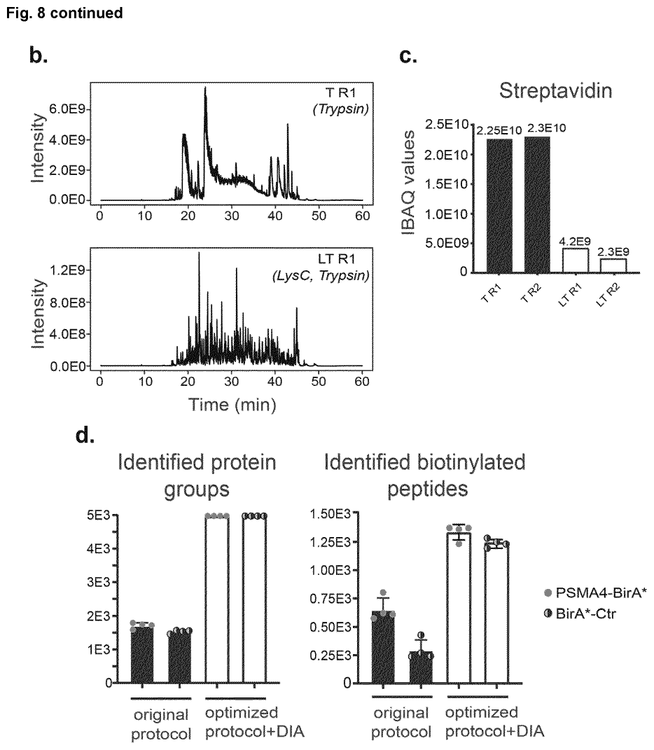

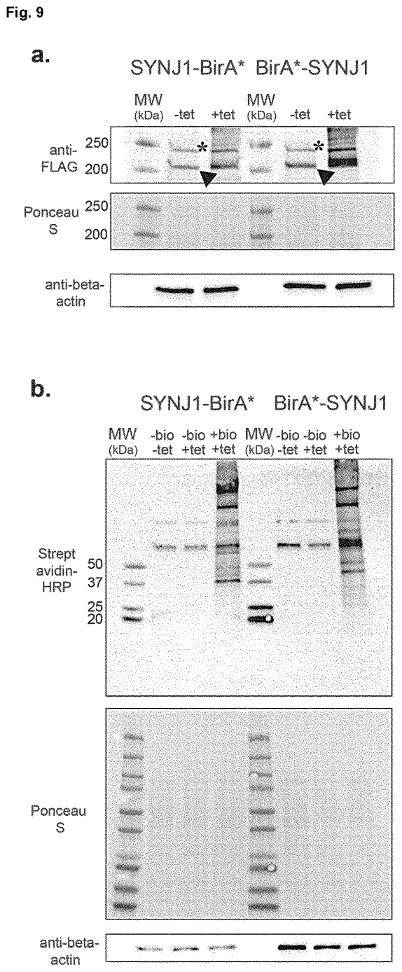

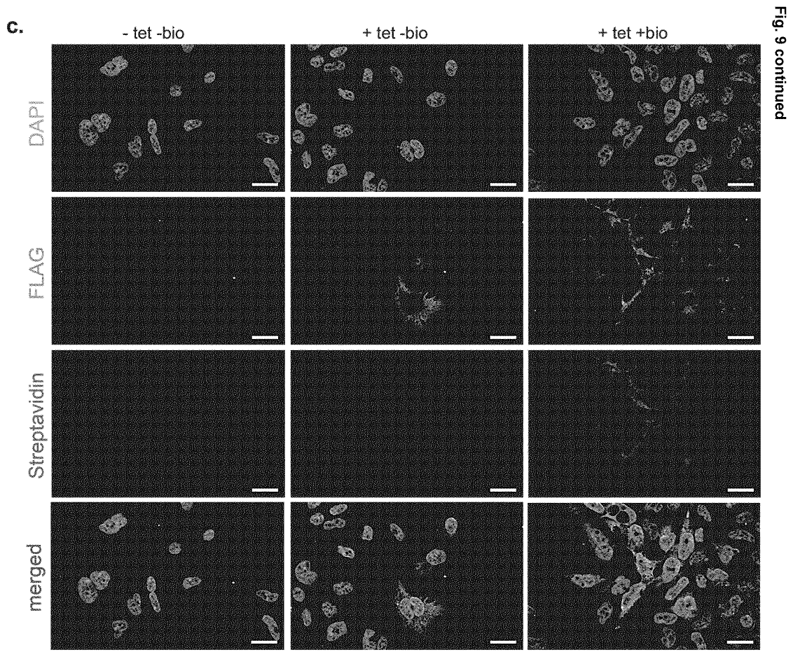

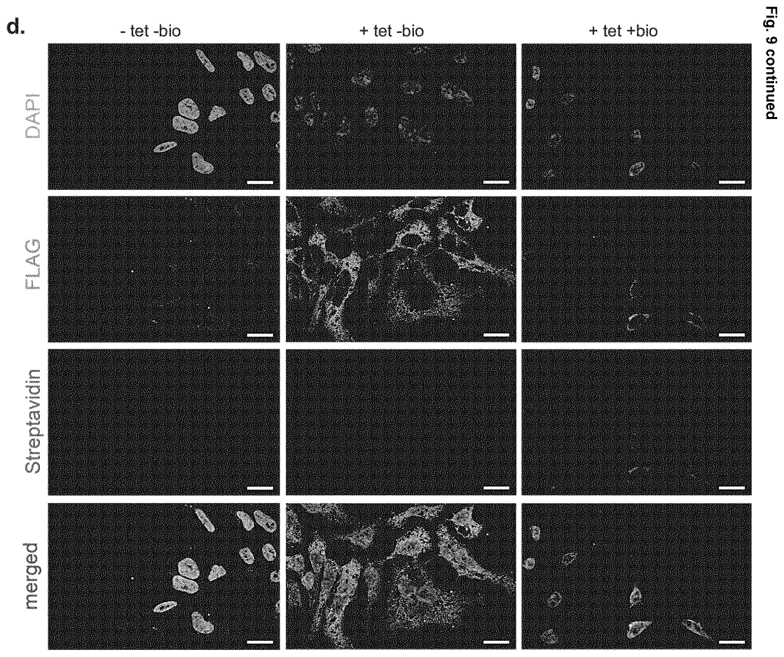

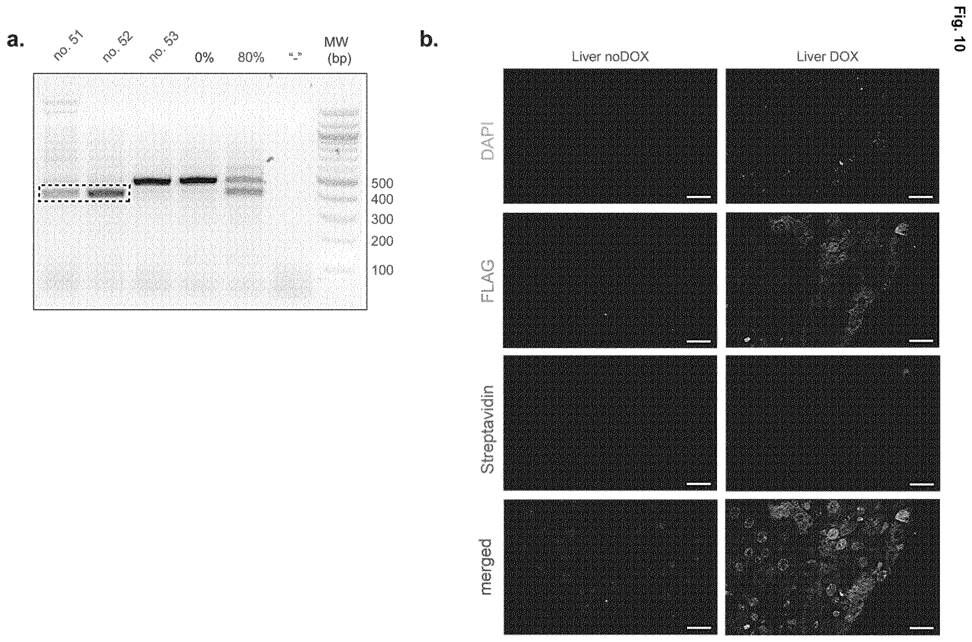

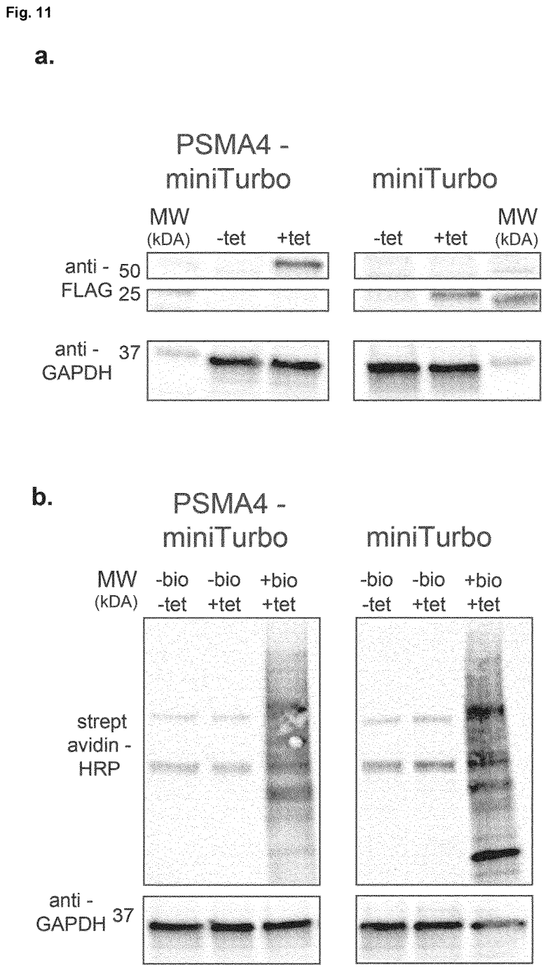

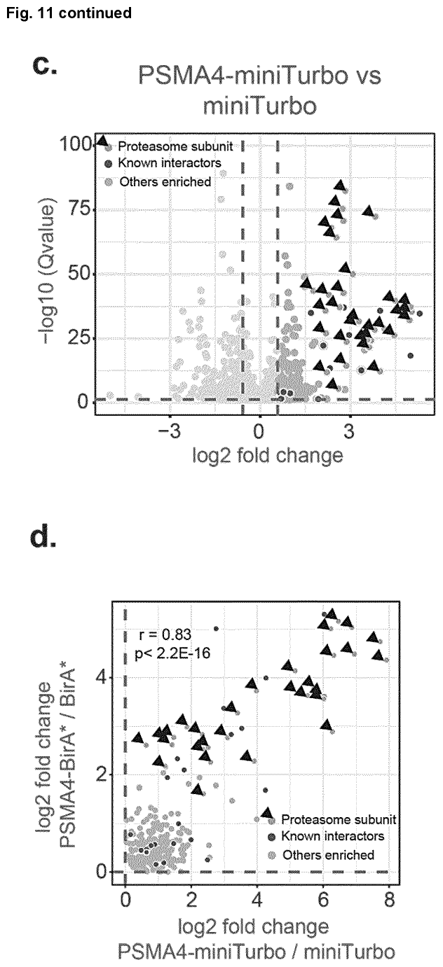

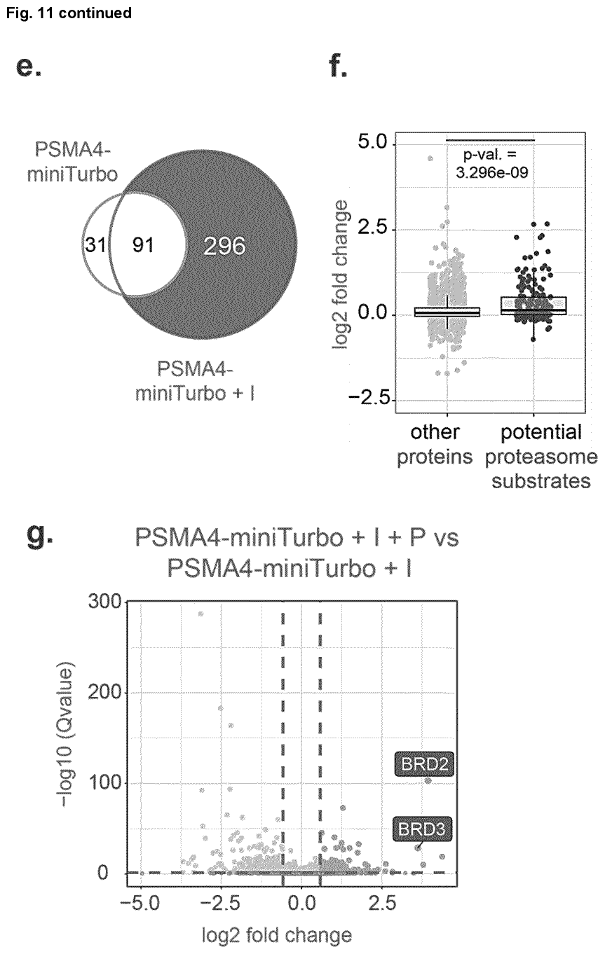

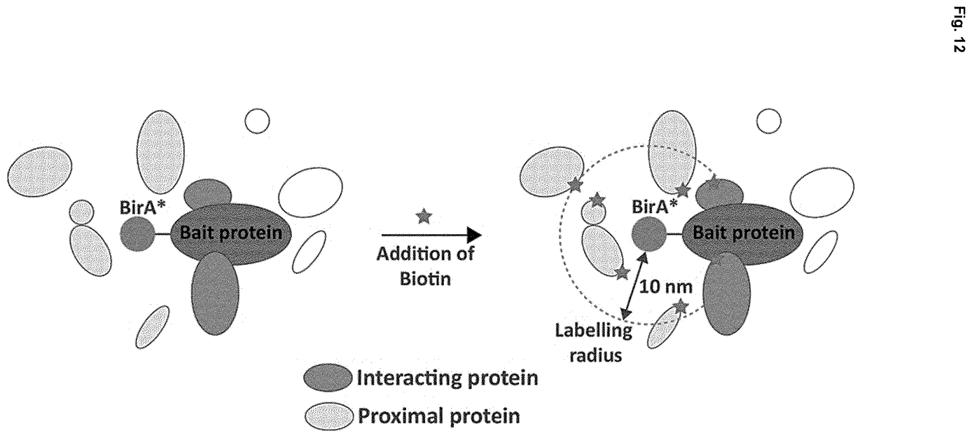

Since the proteasome consists of two distinct sub-complexes (20S core and 19S regulatory particle), two cell lines were generated that enabled monitoring the interactions of each sub-complex separately. The promiscuous biotin ligase BirA* was fused at the C-termini of the core particle protein PSMA4 and the regulatory particle proteins PSMC2 and PSMD3 (FIG. 1A). Each construct also contained a FLAG tag for fusion protein detection. The inventors used these constructs to generate stable HEK293 Flpln TREx (HEK293T) cell lines that overexpress the BirA* fusion proteins under the control of a tetracycline inducible promoter. A cell line expressing only the BirA* protein was used as control to account for nonspecific biotinylation. The inventors confirmed tetracycline-dependent expression of PSMA4-BirA*-FLAG, PSMC2-BirA*-FLAG and BirA*-PSMD3-FLAG in the corresponding cell lines by anti-FLAG immunoblot and confirmed biotinylating activity following supplementation of exogenous biotin using streptavidin-HRP blot (FIG. 1B). Results were validated by immunofluorescence analysis (FIGS. 1C and 7A). Since the BirA* fusion proteins were over-expressed using a CMV promoter, it was also confirmed that the abundance levels of PSMA4-BirA*-FLAG are comparable to the ones of the endogenous PSMA4 using an anti-PSMA4 immunoblot (FIG. 1D). To assess the influence of BirA* fusion proteins on proteasome function, we measured proteasome chymotrypsin-like activity in cell lysates from BirA* expressing cell lines in presence or absence of tetracycline. We observed a slight reduction of proteasome activity (~15-20%) following addition of tetracycline that was comparable between cell lines expressing proteasome BirA* fusion proteins and BirA* control (FIG. 1E). The inventors therefore concluded that the slight reduction in proteasome activity observed likely results from the overexpression of the construct proteins rather than from the interference with the normal proteasome function by the fusion of BirA* to proteasome subunits. To confirm the correct assembly of BirA* fusion proteins into proteasome complexes, Size Exclusion Chromatography coupled to quantitative mass spectrometry (SEC-MS) analysis was performed of the cell line expressing PSMA4-BirA*-FLAG following induction by tetracycline. Protein elution profiles built from mass spectrometry data using the estimated abundance of proteins in each fraction revealed three major distinct peaks corresponding to the major assembly states of the proteasome. These include 30S proteasomes, containing a core particle capped with 2 regulatory particles, 26S proteasomes, containing a core particle capped with 1 regulatory particle, and isolated core particles (20S proteasomes) (FIG. 1F). With the exception of a peak in lower molecular weight fractions, likely representing intermediate complex assemblies, most of the BirA* signal correlated with the elution profile of other proteasome components in all the assembly states, indicating correct incorporation of the PSMA4-BirA* fusion protein in assembled proteasome complexes (FIG. 1F).

### The Present Method Retrieves Proteasome Subunits and Known Proteasome Interactors

In order to identify biotinylated proteins by tagged proteasomes, the inventors optimized a protocol, termed “BioID”, which has been developed previously by Mackmull et al, 2017. Briefly, the protocol entails capture of biotinylated proteins from cell lysates using streptavidin beads followed by enzymatic on bead digestion and analysis of digested peptides by liquid chromatography tandem mass spectrometry (LC-MS/MS)(FIG. 8A). Chemical modification of streptavidin beads was newly introduced by the inventors and they changed the protease digestion strategy to reduce streptavidin contamination following on beads digestion (FIG. 8A). In addition, Data Independent Acquisition (DIA) was newly implemented by the inventors for the analysis of the resulting peptides by mass spectrometry. These optimizations surprisingly allowed to drastically reduce (>4 fold) the background from streptavidin-derived peptides (FIG. 8B), and to increase more than 2-fold the number of identified proteins and biotinylated peptides in BioID experiments (FIG. 8C).

Using this optimized protocol according to one embodiment of the present invention, the inventors analyzed samples enriched from four biological replicates of cell lines expressing PSMA4-BirA*, PSMC2-BirA*, BirA*-PSMD3 or BirA* control. Principal component analysis (PCA) showed clear separation between those 3 different groups (FIG. 2A). Volcano plots highlighted proteasome subunits and known interactors showing strong levels of enrichment (typically >4 fold) compared to BirA*-expressing control cell line (FIG. 2B). By comparing our data to the known proteasome structure (Chen et al. 2016), the inventors could show near to complete coverage of proteasome components and, expectedly, differential levels of enrichment for the 19S or 20S particle depending on the tagged proteasome member (FIG. 2C). The inventors also demonstrated the specificity of protein biotinylation by confirming that 25 out of 26 residues identified as biotinylated by PSMA4-BirA* indeed are less than 10 nm away from the bait protein on the proteasome structure (FIG. 2D).

Next, the inventors investigated the retrieval of known interacting proteins using the following two approaches. First, the inventors compared the BioID data from PSMA4-BirA* to the SEC-MS data obtained from the same cell line. The results show that proteins significantly enriched in BioID display higher correlation of SEC-MS elution profiles with the bait protein PSMA4 than not-enriched proteins (FIG. 2E). Second, the inventors compared the proteins identified as enriched either in PSMA4-BirA* or PSMC2-BirA* to an independent study based on protein correlation profiling performed in different cell types (Fabre et al. 2015). This analysis showed that the present method is able to retrieve 70% (51 out 73) of the proteasome interacting proteins identified in a study, but, in addition, further novel candidate interaction partners (FIG. 2F).

Synaptojanin 1 and Other Phospho-Inositol Phosphatases Interact with the Proteasome

Beyond known proteasome interacting partners and proteins involved in ubiquitin cycle, the results of the present example highlighted two groups of proteins that were underrepresented in previous studies: proteins involved in vesicular trafficking and phospholipid metabolism (FIG. 3A, 3B). Hits belonging to both of these groups were present in the top 125 interactors of both core and regulatory particles. Among these proteins, SYNJ1 (also known as PARK20) is a phospho-inositol phosphatase with a regulatory role in clathrin-mediated endocytosis (Haffner et al., 1997; Mani et al., 2007; Drouet & Lesage, 2014; Soda et al., 2012) that has not been described as a proteasome interactor previously. It was possible with the present method to detect this protein as significantly enrichment from both PSMA4-BirA* and PSMC2-BirA* cell lines (FIG. 3C). In addition, SEC-MS data showed significant co-elution between PSMA4 and SYNJ1 proteins (FIG. 3D), independently supporting a potential interaction between assembled proteasomes and SYNJ1. Closer examination of the present dataset revealed other phospho-inositol phosphatases to be enriched in both PSMA4-BirA* and PSMC2-BirA* BioID samples, suggesting a broader interaction of the proteasome with this class of enzymes (FIG. 3E).

To validate this potential novel interaction, the inventors used two independent strategies. First, they designed a reciprocal biotin proximity-labelling experiment by tagging SYNJ1 with BirA* either N- or C-terminally. The inventors verified expression of the fusion proteins with anti-FLAG immunoblot (FIG. 9A) and biotinylation efficiency using Streptavidin-HRP on lysates from the generated cell lines (FIG. 9B) and confirmed the results using immunofluorescence (FIG. 9C). Next, the inventors applied the optimized BioID protocol to cell lines expressing SYNJ1 fused to BirA* and compared them to the BirA* control line. The inventors could retrieve known SYNJ1 interacting proteins, e.g., SNX9, SH3GL1, SH3GL2, SH3KBP1, and other proteins involved in vesicular trafficking, e.g., CD2AP, GOLGA4, ITSN2, SH3GLB2 (FIG. 4A). Among the proteins significantly enriched in both SYNJ1-BirA* and BirA*-SYNJ1 BioID experiments relatively to BirA* control line, the inventors identified a subset of proteasome subunits from both 20S and 19S particles (FIG. 4B). Consistently, direct comparison of proteasome and SYNJ1 interactomes revealed a shared network of interacting proteins involved in membrane trafficking (FIG. 4C).

As a second independent approach, the inventors used proximity ligation assay (PLA) (Fredriksson et al., 2002) using antibodies against the 20S proteasome a-subunits 1-7 and SYNJ1. The inventors performed PLA in HEK293T cells expressing PSMA4-BirA* as well as wild type U2OS cells. The latter was included to test interaction between endogenous proteasomes and SYNJ1. The inventors could detect PLA signal in both PSMA4-BirA* expressing cells as well as wild type U2OS cells, while the respective negative controls showed minimal or absent background signal (FIG. 4D). These results confirmed in situ proximity between SYNJ1 and the proteasome.

### Example 2

### a Mouse Model for In Vivo Application of the Present Method

Having established and validated proximity labeling of proteasomes in a cell culture model, the inventors designed a strategy to implement the present method in a mouse model (FIG. 5A). The mouse model was designed to express the 20S proteasome core particle PSMA4 fused to the biotinylating enzyme miniTurbo and a FLAG tag for the detection of the fusion protein. The inventors chose miniTurbo instead of BirA* because of its higher biotinylating efficiency of miniTurbo (Branon et al., 2018). PSMA4-miniTurbo was inserted in the Col1a1 locus downstream of a tetracycline responsive element (TRE) in the D34 mouse embryonic stem cell line (Dow et al. 2014). This line carries a cassette encoding the rTA3 transactivator and the fluorescent protein mKate on the Rosa 26 locus under the control of a CAG promoter. Importantly, a LoxP-stop-LoxP cassette is present between the CAG promoter and the rTA3 and mKate expressing cassette, enabling tissue-specific expression via crossing to specific CRE lines. The engineered D34 line was used to generate a mouse line via blastocyst injection. For proof of concept, the inventors crossed the TRE-Psma4-miniTurbo;Rosa26-CAGs-RIK line with a CMV-Cre line that expresses constitutively the CRE recombinase in all tissues (Nagy, 2000). After confirming successful excision of the lox-stop-lox cassette (FIG. 10A), the obtained TRE-Psma4-miniTurbo;Rosa26-CAGs-RIK line was back crossed to C57BL6/J to remove the CMV-Cre allele. The obtained mouse line constitutively expresses the rTA3 transactivator, thereby enabling doxycycline inducible expression of the PSMA4-miniTurbo construct in all tissues.

The inventors assigned eight animals of the desired genotype to two experimental groups: treatment group and a control group (FIG. 5B). The treatment group was fed doxycycline-containing food throughout the 14 days experiment to induce the expression of PSMA4-miniTurbo. The control group was fed regular chow. After a week of this regime, both groups were submitted to daily subcutaneous injections of biotin for a week. The inventors chose seven days of doxycycline induction prior to biotin injection based on the average half-life of proteasomes estimated in vivo (5 days) (Heink et al. 2005). The inventors observed neither significant changes in body weights nor any sign of suffering in both experimental groups for the duration of the experiment (FIG. 5C). Following treatment, animals were sacrificed, and liver, brain, heart, skeletal muscle, and kidney were collected for further analysis.

The inventors first analyzed total proteomes from the collected organs by DIA mass spectrometry. By comparing the relative abundance of peptides derived from miniTurbo, they confirmed successful induction of PSMA4-miniTurbo in all the organs following feeding with doxycycline-containing food (FIG. 5D). The induction of PSMA4-miniTurbo did not alter the total levels of PSMA4 proteins, suggesting compensation of the endogenous protein, as observed in HEK293T cells (FIG. 1D). However, the level of induction varied between organs. By comparing estimates of absolute protein abundance derived from the mass spectrometry data, it could be estimated that in kidney the levels of the tagged PSMA4-miniTurbo varied between 69.7% of the total PSMA4 and 8.2% in the heart (FIG. 5D), likely due to differences in turnover of proteasomes in different organs. The inventors also confirmed successful increase of protein biotinylation by immune-histochemistry analysis of liver tissue following PSMA4-miniTurbo induction and biotin supplementation (FIG. 10B).

Mass spectrometry analysis of biotinylated proteins revealed successful enrichment of proteasome components and known interacting proteins in all organs following feeding with doxycycline-containing food (FIG. 5E). However, the number of bona-fide proteasome-interacting proteins identified and their enrichment (log 2 fold change) varied among organs, being most prominent in kidney and liver and especially low in the brain. This was in line with the different absolute levels of PSMA4-miniTurbo detected by whole organ proteome analysis (FIG. 5D). The inventors next compared the candidate proteasome-interacting proteins identified in HEK293T cells and mouse organs and related them to previously reported interactors (FIG. 5F). The inventors were able to identify a significant overlap between the HEK293T and mouse interactomes and retrieved >50% of the previously reported proteasome-interacting proteins. In addition, they detected 136 potential novel candidate proteasome interacting proteins that were consistently identified both in vitro and in vivo, including the phospho-inositol phosphatase INPP5B and the adapter protein ITSN2, which were also found to be shared interactors between proteasomes and SYNJ1 in cultured cells. Together these data demonstrate that proteasome interacting proteins can be quantified from mouse organs using the present method.

### Identification of Proteasome Substrates by the Present Method

Having demonstrated that the method according to the invention can be used both in cultured cells and in vivo to obtain snapshots of the proteasome interactome, the inventors next investigated whether they could use this approach directly to identify proteasome substrates. The inventors reasoned that under steady state conditions the interaction between proteasomes and their substrates might be too short lived to enable efficient biotinylation. Therefore, they generated HEK293T cells expressing PSMA4-miniTurbo enabling shorter biotinylation time (2 vs. 24 hours) thanks to the enhanced activity of miniTurbo as compared to BirA* (Branon et al., 2018) (FIG. 11A). They confirmed enrichment of proteasome members and interacting proteins (FIG. 11B) and observed positive correlation between the enrichments measured using PSMA4-miniTurbo and PSMA4-BirA*, relatively to their respective control lines (FIG. 11C).

Next, the inventors included a step of acute inhibition of the proteasome by a potent cell-permeable inhibitor MG132 for 4 hours prior to biotin supplementation (FIG. 6A). Principal component analysis revealed a clear impact of proteasome inhibition on the biotinylated proteins quantified by mass spectrometry (FIG. 6B). Direct comparison of pull downs from cells treated with MG132 versus control cells revealed prominent enrichment of proteasome activators and ubiquitin (FIG. 6C), consistent with the recruitment of proteasome activators and direct ubiquitination of proteasome members following inhibition (Rechsteiner & Hill, 2005). To identify potential proteasome substrates, they focused on a subset of 296 proteins that were enriched relative to miniTurbo control only in presence of MG132 (FIG. 11D). Among these, the inventors found well characterized proteasome substrates including the transcription factors ATF4, HIF1A, JUN and MYC, proteins mediating ER stress response such as XBP1, as well as cell-cycle regulated proteins such as CDC7, CDT1 and CDKN2A (FIGS. 6D and 11E).

### Example 3

Finally, the inventors investigated whether they could use the same strategy to identify selective induction of protein degradation by small molecules. For this purpose, they used KB02-JQ1, a well-characterized Proteolysis targeting chimera (PROTAC) that targets bromodomain-containing proteins (BRDs) for proteasomal degradation (Zhang et al. 2019). They pre-treated cells with KB02-JQ1 for 8 hours prior to proteasome inhibition and biotin supplementation (FIG. 6E). Mass spectrometry analysis showed no global changes induced by KB02-JQ1 either in presence or absence of MG132, as indicated by PCA (FIG. 6F). However, the inventors could detect prominent enrichment of BRD containing proteins following treatment with KB02-JQ1 (FIG. 11F). The effect was more pronounced for BRD2 and BRD3 and less striking for BRD4 (FIG. 6G), presumably reflecting different kinetics of induced degradation by KB02-JQ1 (Zhang et al. 2019). Notably, the enrichment of BRD2 and BRD3 was also detectable in absence of MG132, in contrast to endogenous substrates that become enriched only following proteasome inhibition (FIG. 6D).

Together, these data demonstrate that the method according to the invention can be used to detect both endogenous and protein degrader-induced substrates of the proteasome in cultured cells.

### REFERENCES

Branon, T. C., Bosch, J. A., Sanchez, A. D., Udeshi, N. D., Svinkina, T., Carr, S. A., Feldman, J. L., Perrimon, N., & Ting, A. Y. (2018). Efficient proximity labeling in living cells and organisms with TurboID. Nature biotechnology, 36(9), 880-887. https://doi.org/10.1038/nbt.4201
Chen, S., Wu, J., Lu, Y., Ma, Y. B., Lee, B. H., Yu, Z., Ouyang, Q., Finley, D. J., Kirschner, M. W., & Mao, Y. (2016). Structural basis for dynamic regulation of the human 26S proteasome. Proceedings of the National Academy of Sciences of the United States of America, 113(46), 12991-12996. https://doi.org/10.1073/pnas.1614614113
Cox, J., & Mann, M. (2008). MaxQuant enables high peptide identification rates, individualized p.p.b.-range mass accuracies and proteome-wide protein quantification. Nature biotechnology, 26(12), 1367-1372. https://doi.org/10.1038/nbt.1511
Dow, L. E., Nasr, Z., Saborowski, M., Ebbesen, S. H., Manchado, E., Tasdemir, N., Lee, T., Pelletier, J., & Lowe, S. W. (2014). Conditional reverse tet-transactivator mouse strains for the efficient induction of TRE-regulated transgenes in mice. PloS one, 9(4), e95236. https://doi.org/10.1371/journal.pone.0095236
Fabre, B., Lambour, T., Garrigues, L., Amalric, F., Vigneron, N., Menneteau, T., Stella, A., Monsarrat, B., Van den Eynde, B., Burlet-Schiltz, O., & Bousquet-Dubouch, M. P. (2015). Deciphering preferential interactions within supramolecular protein complexes: the proteasome case. Molecular systems biology, 11(1), 771. https://doi.org/10.15252/msb.20145497
Mackmull, M. T., Klaus, B., Heinze, I., Chokkalingam, M., Beyer, A., Russell, R. B., Ori, A., & Beck, M. (2017). Landscape of nuclear transport receptor cargo specificity. Molecular systems biology, 13(12), 962. https://doi.org/10.15252/msb.20177608
Miyazaki, J., Takaki, S., Araki, K., Tashiro, F., Tominaga, A., Takatsu, K., & Yamamura, K. (1989). Expression vector system based on the chicken beta-actin promoter directs efficient production of interleukin-5. Gene, 79(2), 269-277. https://doi.org/10.1016/0378-1119(89)90209-6
Nagy A. (2000). Cre recombinase: the universal reagent for genome tailoring. Genesis (New York, N.Y.: 2000), 26(2), 99-109.
Roux K J, Kim D I, Raida M, Burke B. A promiscuous biotin ligase fusion protein identifies proximal and interacting proteins in mammalian cells. J Cell Biol. 2012 Mar. 19; 196(6):801-10. doi: 10.1083/jcb.201112098. Epub 2012 Mar. 12. PMID: 22412018; PMCID: PMC3308701.
Pettersen, E. F., Goddard, T. D., Huang, C. C., Couch, G. S., Greenblatt, D. M., Meng, E. C., & Ferrin, T. E. (2004). UCSF Chimera—a visualization system for exploratory research and analysis. Journal of computational chemistry, 25(13), 1605-1612. https://doi.org/I0.1002/jcc.20084
Rechsteiner, M., & Hill, C. P. (2005). Mobilizing the proteolytic machine: cell biological roles of proteasome activators and inhibitors. Trends in cell biology, 15(1), 27-33. https://doi.org/10.1016/j.tcb.2004.11.003
Samavarchi-Tehrani, P., Samson, R., Gingras, A.-C. Proximity Dependent Biotinylation: Key Enzymes and Adaptation to Proteomics Approaches*, Molecular & Cellular Proteomics, Volume 19, Issue 5, 2020, Pages 757-773, ISSN 1535-9476, https://doi.org/10.1074/mcp.R120.001941.
Storey, J. D. (2002), A direct approach to false discovery rates. Journal of the Royal Statistical Society: Series B (Statistical Methodology), 64: 479-498. https://doi.org/10.1111/1467-9868.00346
Zhang, X., Crowley, V. M., Wucherpfennig, T. G., Dix, M. M., & Cravatt, B. F. (2019). Electrophilic PROTACs that degrade nuclear proteins by engaging DCAF16. Nature chemical biology, 15(7), 737-746. https://doi.org/10.1038/s41589-019-0279-5

1. A method of detecting an interaction or proximity between a fusion protein and a target protein, comprising
a. providing a fusion protein and/or a recombinant nucleic acid molecule encoding said fusion protein, said fusion protein comprising a proteasome complex protein fused to a promiscuous biotin ligase,
b. inhibiting the enzyme function of the fusion protein or the enzyme complex,
c. biotinylating a target protein that interacts with and/or is in proximity to the fusion protein,
d. enriching the biotinylated target protein, and
e. identifying the isolated target protein using mass spectrometry (MS).

2. The method according to claim 1, wherein the promiscuous biotin ligase is a E. coli or B. subtilis BirA biotin ligase or variant thereof in its integral or split form.

3. The method according to claim 2, wherein the promiscuous biotin ligase is selected from the group consisting of BirA* (BirA(R118G)), BioID, BioID2, BASU, AirID, microID, microID2, ultraID, TurboID, miniTurbo, APEX and APEX2.

4. The method according to claim 1, wherein the proteasome complex protein is selected from the group consisting of an 11S proteasome activator, PA200/Blm10 proteasome activator, PAN protein complex, ARC/Mpa protein complex, 19S, 20S, 26S, and 30S proteasome particle and/or wherein the proteasome complex protein is selected from the group consisting of PSMA1-8, PSMB1-11, PSMC1-6, PSMD1-14, PSME1-4, PSMF1, PAMD12, ADRM1, USP14/Ubp6, UCHL5/UCH37, UBE3C/KIAA10/Hul5, and SHFM1/DSS1, or any combination thereof.

5. The method according to claim 1, wherein the target protein to be identified is a substrate or interaction partner of the proteasome.

6. The method according to claim 1, comprising additionally supplementing biotin or derivates thereof before the biotinylation step c. of claim 1.

7. The method according to claim 1, wherein when step a. of claim 1 comprises a recombinant nucleic acid sequence encoding said fusion protein, expression of said nucleic acid molecule is under control of an inducible promoter.

8. The method according to claim 7, comprising additionally treating the cell in which said fusion protein is expressed with at least one candidate substance.

9. The method according to claim 1, wherein inhibiting the enzyme function of the fusion protein or the enzyme complex comprises administering a protease inhibitor compound.

10. The method according to claim 1, wherein the fusion protein further comprises a protein- or affinity-tag.

11. The method according to claim 1, comprising
a. providing a recombinant nucleic acid molecule encoding the fusion protein, said fusion protein comprising a proteasome complex protein fused to a promiscuous biotin ligase selected from the group consisting of BirA*(BirA(R118G)), BioID, BioID2, BASU, AirID, microID, microID2, ultraID, TurboID, miniTurbo, APEX and APEX2,
b. expressing the fusion protein,
c. inhibiting of protease function of the fusion protein or proteasome complex,
d. supplementing biotin or derivates thereof,
e. biotinylating a target protein that is in proximity to and/or interacts with the fusion protein,
f. enriching the biotinylated target protein, and
g. identifying the isolated target protein using quantitative mass spectrometry.

12. A nucleic acid molecule encoding a fusion protein comprising a proteasome complex protein fused to a promiscuous biotin ligase selected from the group consisting of BirA* (BirA(R118G)), BioID, BioID2, BASU, AirID, microID, microID2, ultraID, TurboID and miniTurbo.

13. A fusion protein comprising a proteasome complex protein fused to a promiscuous biotin ligase selected from the group consisting of BirA*(BirA(R118G)), BioID, BioID2, BASU, AirID, microID, microID2, ultraID, TurboID and miniTurbo.

14. A genetically modified cell comprising the nucleic acid according to claim 12.

15. A genetically modified mouse comprising the nucleic acid molecule according to claim 12 in at least one tissue.

16. A kit comprising the nucleic acid molecule according to claim 12, and one or more of:
a. means for cell-transfection, transduction, infection or gene knock-in,
b. doxycycline or tetracycline,
c. biotin, and/or
d. a protease-inhibitor.

17. The method according to claim 1, for determining an effect of a candidate substance on the interaction and/or proximity between a proteasomal complex protein and a target protein.

18. The method according to claim 7, wherein the inducible promoter is controlled by administration of doxycycline or tetracycline, and the nucleic acid comprises a doxycycline/tetracycline-controlled Tet-Off and Tet-On gene expression system.

19. The method according to claim 8, wherein the at least one candidate substance is administered prior to biotin supplementation.

20. The method according to claim 9, wherein the protease inhibitor compound is administered at least 1 to 4 hours prior to, or for 1 to 24 hours during biotin supplementation.


SEQUENCE LISTING (4 sequences)

SEQ ID NO: 1 - protein, 612 aa, synthetic construct
MSRRYDSRTTIFSPEGRLYQVEYAMEAIGHAGTCLGILANDGVLLAAERRNIHKLLDEVFFSEKIYKLNEDMACSVAGITSDANVLTNELRLIAQRYLLQYQEPIPCEQLVTALCDIKQAYTQFGGKRPFGVSLLYIGWDKHYGFQLYQSDPSGNYGGWKATCIGNNSAAAVSMLKQDYKEGEMTLKSALALAIKVLNKTMDVSKLSAEKVEIATLTRENGKTVIRVLKQKEVEQLIKKHEEEEAKAEREKKEKEQKEKDKYPTFLYKVVDSRMKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGGRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEKGGSGPGGGAPDYKDDDDK

SEQ ID NO: 2 - protein, 784 aa, synthetic construct
MPDYLGADQRKTKEDEKDDKPIRALDEGDIALLKTYGQSTYSRQIKQVEDDIQQLLKKINELTGIKESDTGLAPPALWDLAADKQTLQSEQPLQVARCTKIINADSEDPKYIINVKQFAKFVVDLSDQVAPTDIEEGMRVGVDRNKYQIHIPLPPKIDPTVTMMQVEEKPDVTYSDVGGCKEQIEKLREVVETPLLHPERFVNLGIEPPKGVLLFGPPGTGKTLCARAVANRTDACFIRVIGSELVQKYVGEGARMVRELFEMARTKKACLIFFDEIDAIGGARFDDGAGGDNEVQRTMLELINQLDGFDPRGNIKVLMATNRPDTLDPALMRPGRLDRKIEFSLPDLEGRTHIFKIHARSMSVERDIRFELLARLCPNSTGAEIRSVCTEAGMFAIRARRKIATEKDFLEAVNKVIKSYAKFSATPRYMTYNCPTFLYKVVDSRMKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGGRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEKGGSGPGGGAPDYKDDDDK

SEQ ID NO: 3 - protein, 885 aa, synthetic construct
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGGRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEKGGSGPGGGAPDYKDDDDKLGAPTSLYKKVGMKQEGSARRRGADKAKPPPGGGEQEPPPPPAPQDVEMKEEAATGGGSTGEADGKTAAAAAEHSQRELDTVTLEDIKEHVKQLEKAVSGKEPRFVLRALRMLPSTSRRLNHYVLYKAVQGFFTSNNATRDFLLPFLEEPMDTEADLQFRPRTGKAASTPLLPEVEAYLQLLVVIFMMNSKRYKEAQKISDDLMQKISTQNRRALDLVAAKCYYYHARVYEFLDKLDVVRSFLHARLRTATLRHDADGQATLLNLLLRNYLHYSLYDQAEKLVSKSVFPEQANNNEWARYLYYTGRIKAIQLEYSEARRTMTNALRKAPQHTAVGFKQTVHKLLIVVELLLGEIPDRLQFRQPSLKRSLMPYFLLTQAVRTGNLAKFNQVLDQFGEKFQADGTYTLIIRLRHNVIKTGVRMISLSYSRISLADIAQKLQLDSPEDAEFIVAKAIRDGVIEASINHEKGYVQSKEMIDIYSTREPQLAFHQRISFCLDIHNMSVKAMRFPPKSYNKDLESAEERREREQQDLEFAKEMAEDDDDSFP

SEQ ID NO: 4 - protein, 544 aa, synthetic construct
MSRRYDSRTTIFSPEGRLYQVEYAMEAIGHAGTCLGILANDGVLLAAERRNIHKLLDEVFFSEKIYKLNEDMACSVAGITSDANVLTNELRLIAQRYLLQYQEPIPCEQLVTALCDIKQAYTQFGGKRPFGVSLLYIGWDKHYGFQLYQSDPSGNYGGWKATCIGNNSAAAVSMLKQDYKEGEMTLKSALALAIKVLNKTMDVSKLSAEKVEIATLTRENGKTVIRVLKQKEVEQLIKKHEEEEAKAEREKKEKEQKEKDKYPTFLYKVVDSRMASIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAAMLIRELRAALELFEQEGLAPYLSRWEKLDNFINRPVKLIIGDKEIFGISRGIDKQGALLLEQDGVIKPWMGGEISLRSAEKLQDDYKDDDDK